(19) United States
(12) Patent Application Publication
(10) Pub. No.: US 2003/0148421 A1
(43) Pub. Date: Aug. 7, 2003

(54) Gene products that regulate glucose response in cells

(76) Inventors: Christopher B. Newgard, Dallas, TX (US); Per Bo Jensen, Ballerup (DK)

Correspondence Address: Steven L. Highlander, Fullbright & Jaworski L.L.P., Suite 2400, 600 Congress Avenue, Austin, TX, 78701, US

(21) Appl. No.: 10/080,381

(22) Filed: Feb. 19, 2002

Related U.S. Application Data
(60) Provisional application No. 60/270,251, filed on Feb. 20, 2001.
(60) Provisional application No. 60/274,706, filed on Mar. 9, 2001.
(60) Provisional application No. 60/291,354, filed on May 15, 2001.

(51) Int. Cl. C12P 21/02; C12N 5/06; C07K 14/47; C12N 1/21; C07H 21/04; C12N 9/00

(52) U.S. Cl. 435/69.1; 435/183; 435/320.1; 435/325; 435/252.3; 530/388.26; 530/350; 536/23.2

(57) ABSTRACT

The present invention describes the identification of numerous genes, both known and unknown, that play an important role in the ability of cell to respond to glucose stimulation under physiologic conditions. These genes may be used to enhance, stabilize or introduce glucose-responsiveness in a host cell, in particular, a host cell that secretes insulin. In addition, these genes may be used as targets for drug screening and as diagnostic indicators for the loss of glucose-responsiveness.

This application is related to previously filed U.S. Provisional Application 60/270,251, filed Feb. 20, 2001, and U.S. Provisional Application 60/274,706, filed Mar. 9, 2001, and U.S. Provisional Application 60/291,354, filed May 15, 2001. The entire text of the above-referenced disclosures are specifically incorporated by reference herein without disclaimer.

### BACKGROUND OF THE INVENTION

1. Field of the Invention

The present invention relates generally to the fields of cellular and molecular biology, genetics, endocrinology, and cellular physiology. More particularly, it concerns the identification of various genes that impact glucose response in cells, and their use in the manipulation of glucose-mediated insulin secretion.

2. Description of Related Art

One of the major goals of diabetes research is to create genetically engineered cells that can produce insulin—in effect, an artificial pancreas. The implantation of such cells into a host would obviate the need for costly and inconvenient insulin injections that many diabetics must suffer through. Many engineering efforts towards this end have been successful, to a degree, and include cells that express heterologous insulin, hexokinase, and other genes that are involved in insulin secretion. U.S. Pat. Nos. 5,427,940, 5,744,327, 5,747,325, 5,792,656, 5,993,799, 5,811,266, 6,087,129, 6,110,707, and 6,171,856.

Of particular interest for insulin production are secretory cells, especially neuroendocrine cells, as these cells have endogenous functions that make them uniquely suited for production of secreted peptide hormones. These specialized functions include the regulated secretory pathway. The regulated secretory pathway embodies the secretory granules of neuroendocrine cells which serve as the site of maturation and storage of a large class of peptide hormones with profound biological functions. Proper biological function of the peptides is due both to their secretion in a regulated and titratable manner as well as a complex set of post-translational modifications resulting in the final biologically active product.

While neuroendocrine cell lines derived from tissues other than pancreatic islets often have a fully functional regulated secretory pathway, allowing them, for example, to process proinsulin to mature insulin and store it in secretory granules, the primary limitation of such cells remains their inability to respond to physiologic levels of glucose and, in so doing, secrete insulin in vivo with the same precision as normal pancreatic islet cells. Insulinoma cell lines, derived from pancreatic islet β-cells, are a preferred vehicle for creation of genetically engineered islet cell surrogates, because they are derived from the cell type with the desired regulatory function.

While insulinoma lines provide an advantage in that they can be grown in essentially unlimited quantity at relatively low cost, most exhibit differences in their glucose-stimulated insulin secretory response relative to normal islets (reviewed in Newgard, 1996). These differences can be quite profound, such as in the case of RINm5F cells, which were derived from a radiation-induced insulinoma and which in their current form are completely lacking in any acute glucose-stimulated insulin secretion response (Halban et al., 1983; Shimuzu et al., 1988). RIN 1046-38 cells are also derived from a radiation-induced insulinoma but can be shown to be glucose responsive when studied at low passage numbers (Clark et al., 1990). This response is maximal at subphysiological glucose concentrations and is lost entirely when these cells are cultured for more than 40 passages (Clark et al., 1990). GLUT-2 and glucokinase are expressed in low passage RIN 1046-38 cells but are gradually diminished with time in culture in synchrony with the loss of glucose-stimulated insulin release (Ferber et al., 1994).

Restoration of GLUT-2 and glucokinase expression in RIN 1046-38 cells by stable transfection restores glucose-stimulated insulin secretion (Ferber et al., 1994; Clark et al., 1997; Hohmeier et al., 1997), and the use of these genes as a general tool for engineering of glucose-sensing has been described (U.S. Pat. No. 5,427,940). RIN 1046-38 cells transfected with the GLUT-2 gene alone are maximally glucose responsive at low concentrations of the sugar (approximately 50 μM), but the threshold for response can be shifted by preincubating the cells with 2-deoxyglucose or 5-thioglucose, which when converted to their phosphorylated derivatives inside the cell serve as inhibitors of low Kₘ hexokinase, but not glucokinase activity (Ferber et al., 1994; Hohmeier et al., 1997).

While early lines were derived from radiation- or virus-induced tumors (Gazdar et al., 1980; Santerre et al., 1981), more recent work has centered on the application of transgenic technology (Efrat et al., 1988; Miyazaki et al., 1990). A general approach taken with the latter technique is to express an oncogene, most often SV40 T-antigen, under control of the insulin promoter in transgenic animals, thereby generating β-cell tumors that can be used for propagating insulinoma cell lines (Efrat et al., 1988; Miyazaki et al., 1990).

Similar to insulinoma cells derived by X-irradition, cell lines derived by transgenic expression of T-antigen in β-cells also exhibit variable phenotypes (Efrat et al., 1988; Miyazaki et al., 1990; Whitesell et al., 1991; Efrat et al., 1993). Some lines have little glucose-stimulated insulin release or exhibit maximal responses at subphysiological glucose concentrations (Efrat et al., 1988; Miyazaki et al., 1990; Whitesell et al., 1991), while others respond to glucose concentrations over the physiological range (Miyazaki et al., 1990; Efrat et al., 1993). It appears that the near-normal responsiveness of the latter cell lines is not permanent, since further time in culture results in a shift in glucose dose response such that the cells secrete insulin at subphysiological glucose concentrations (Efrat et al., 1993). In some cases, these changes have been correlated with changes in the expression of glucose transporters and glucose-phosphorylating enzymes. Miyazaki et al. isolated two classes of clones from transgenic animals expressing an insulin promoter/T-antigen construct. Glucose-unresponsive lines such as MIN-7 were found to express GLUT-1 rather than GLUT-2 as their major glucose transporter isoform, while MIN-6 cells were found to express GLUT-2 and to exhibit normal glucose-stimulated insulin secretion (Miyazaki et al., 1990). Efrat and coworkers demonstrated that their cell line βTC-6, which exhibits a glucose-stimulated insulin secretion response that resembles that of the islet in magnitude and concentration dependence, expressed GLUT-2 and contained a glucokinase:hexokinase activity ratio similar to that of the normal islet (Efrat et al., 1993). With time in culture, glucose-stimulated insulin release became maximal at low, subphysiological glucose concentrations. GLUT-2 expression did not change with time in culture, and glucokinase activity actually increased slightly, but the major change was a large (approximately 6-fold) increase in hexokinase expression (Efrat et al., 1993). Furthermore, overexpression of hexokinase I, but not GLUT-1, in well-differentiated MIN-6 cells results in both increased glucose metabolism and insulin release at subphysiological glucose concentrations. Similar results have been obtained upon overexpression of hexokinase I in normal rat islets (Becker et al., 1994b). Finally, INS-1 cells, derived from an X-ray induced insulinoma tumor in media containing 2-mercaptoethanol have been shown to express GLUT-2 and glucokinase as their predominant glucose transporter and glucose phosphorylating enzyme, respectively, consistent with their retention of glucose-stimulated insulin secretion in response to physiologic glucose concentrations (Marie et al., 1993). These results are all consistent with the observations of Ferber, et al. and Hohmeier, et al. described above in showing that a high hexokinase:glucokinase ratio will cause insulin-secreting cells to respond to glucose concentrations less than those required to stimulate the normal β-cell.

There are isolated examples of insulinoma cell lines with more differentiated properties derived by either the X-irradiation or transgenic approaches, including the rat line INS-1, and mouse cell lines such as MIN-6, βTC6-F7, and βHC9. These lines have insulin content closer to that of normal islets and retention of some glucose-stimulated insulin secretion (Asfari et al., 1992; Miyazaki et al., 1990; Knaack et al., 1994; Liang et al., 1996; Noda et al., 1996). However, even the best rodent cell lines are imperfect. For example, INS-1 cells generally exhibit only a 2-4 fold increment in insulin secretion in response to glucose (Asfari et al., 1992; Noel et al., 1997; Antinozzi et al., 1998), far less than the 15-fold responses achievable with freshly isolated primary islets (Zawalich & Zawalich, 1996). Also, MIN-6 cells exhibit secretory responses to pyruvate, which is not a secretagogue for normal islets (Skelly et al., 1998), while βHC9 cells grow very slowly and are thus difficult to study. Finally, loss of differentiated features as a function of time in tissue culture has been reported for several rodent cell lines, including RIN 1046-38, βTC6, and INS-1 (Knaack et al., 1994; Clark et al., 1990; Ferber et al., 1994; Hohmeier et al., 2000). Stable glucose responsiveness has been reported for βTC6 cells after clonal selection in soft agar (Knaack et al., 1994), but even these cloned cell lines (e.g., βTC6-F7) appear to lose glucose responsiveness after prolonged tissue culture (Zhou et al., 1998).

An alternative to insulinoma cell lines are non-islet cell lines of neuroendocrine origin that are engineered for insulin expression. The foremost example of this is the AtT-20 cell, which is derived from ACTH secreting cells of the anterior pituitary. Almost two decades ago, Moore et al. demonstrated that stable transfection of AtT-20 cells with a construct in which a viral promoter is used to direct expression of the human proinsulin cDNA resulted in cell lines that secreted the correctly processed and mature insulin polypeptide (Moore et al., 1983). Insulin secretion from such lines (generally termed AtT-20ins) can be stimulated by agents such as forskolin or dibutyryl cAMP, with the major secreted product in the form of mature insulin. This suggests that these cells contain a regulated secretory pathway that is similar to that operative in the islet β-cell (Moore et al., 1983; Gross et al., 1989).

It also has become clear that the endopeptidases that process proinsulin to insulin in the islet β-cell, termed PC2 and PC3, are also expressed in AtT-20ins cells (Smeekens et al., 1990; Hakes et al., 1991). AtT-20ins cells do not respond to glucose as a secretagogue (Hughes et al., 1991). Interestingly, AtT-20 cells express the glucokinase gene (Hughes et al., 1991; Liang et al., 1991) and at least in some lines, low levels of glucokinase activity (Hughes et al., 1991; 1992; Quaade et al., 1991), but are completely lacking in GLUT-2 expression (Hughes et al., 1991; 1992). Stable transfection of these cells with GLUT-2, but not the related transporter GLUT-1, confers glucose-stimulated insulin secretion, albeit with maximal responsiveness at subphysiological glucose levels, probably because of a non-optimal hexokinase:glucokinase ratio (Hughes et al., 1992; 1993).

The studies with AtT-20ins cells are important because they demonstrate that neuroendocrine cell lines that normally lack glucose-stimulated peptide release may be engineered for this function. Other cell lines that are characterized as neuroendocrine, but lacking in endogenous glucose response include PC12, a neuronal cell line (ATCC CRL 1721) and GH3, an anterior pituitary cell line that secretes growth hormone (ATCC CCL82.1). These lines exhibit important properties such as a regulated secretory pathway, expression of endopeptidases required for processing of prohormones to their mature hormone products, and post-translational modification enzymes. Thus, many neuroendocrine cell lines could be useful for delivering insulin if the remaining problem of glucose responsiveness could be addressed.

In sum, while certain genetic engineering tools have become available for conferring or improving glucose-stimulated insulin secretion in neuroendocrine cell lines, including insulinoma cells, there may be other, as yet undiscovered gene products that may complement or enable maximal effectiveness of said tools. In a recent study, a cellular model for unearthing all of the genes involved in glucose-stimulated insulin secretion has been described (Hohmeier et al., 2000; Chen, et al., 2000). This involved the stable transfection of INS-1 cells with a plasmid containing the human proinsulin gene and the neomycin (G418) resistance gene. After selection with G418 and clonal expansion, 67% of the resultant clones were found to be poorly responsive to glucose in terms of insulin secretion (≦2-fold stimulation by 15 mM compared to 3 mM glucose), 17% of the clones were moderately responsive (2-5-fold stimulation), and 16% were strongly responsive (5-13-fold stimulation). The differences in responsiveness could not be ascribed to differences in insulin content. Detailed analysis of one of the strongly responsive lines (832/13), revealed that its potent response to glucose (average of 10-fold) was stable over 66 population doublings (approximately 7.5 months of tissue culture), with half-maximal stimulation at 6 mM glucose, similar to the response of normal islets. Furthermore, in the presence of 15 mM glucose, several agents known to potentiate glucose-stimulated insulin secretion in normal islets were shown to be similarly effective when applied to 832/13 cells, including IBMX, a mixture of fatty acids (oleate/palmitate), and the hormone glucagon-like peptide-1 (GLP-1). Glucose-stimulated insulin secretion was also potentiated by the sulfonylurea tolbutamide, and abolished by diazoxide, demonstrating the operation of the ATP-sensitive K⁺-channel (K_{ATP}) in 832/13 cells.

Subsequent analysis revealed that several other INS-1 derived clones (833/15 and 834/40) exhibited similar robust function as described for the 832/13 line (Hohmeier et al., 2000; Chen et al., 2000). Furthermore, several glucose unresponsive clones were also isolated and characterized, including 832/1, 832/2, 834/105, and 834/112. This set of glucose responsive and unresponsive clones has become a key tool for defining genes that determine glucose responsiveness in β-cells as described in this application.

Recent studies from multiple laboratories have revealed that development of the pancreatic islets and the specialized insulin-producing islet β-cells requires the concerted action of multiple transcription factors such as Pdx-1, Isl1, Beta2 (NeuroD), Pax4, Pax6, Nkx 2.2, and Nkx 6.1 (Jonsson et al., 1994; Offield et al., 1996; Ahlgren et al., 1997; Sander et al., 1997; Sosa-Pineda et al., 1997; St. Onge et al., 1997; Naya et al., 1997; Sussel et al., 1998; Edlund, 1998; Sander & German, 1997; Sander et al., 1998; Mirmira et al., 2000). These transcription factors are expressed at various stages of pancreas and 10 islet development, and ablation of their expression by homolgous recombination (gene knock-out) blocks or impairs normal islet development. Thus, one potential strategy for improving performance of genetically engineered cell lines might be to express transcription factors implicated in β-cell development in candidate insulinoma or other neuroendocrine cell lines. U.S. Pat. No. 6,127,598. However, successful conversion of candidate cell lines to a stable and fully differentiated β-cell phenotype by expression of a transcription factor has not yet been accomplished, suggesting that co-expression of combinations of genes, including some as yet undiscovered, may be required for achievement of the ultimate goal.

Glucose-stimulated insulin secretion is potentiated by products of the preproglucagon gene, including glucagon and glucagon-like peptide-1 (GLP-1). Human islet cells express both the discrete glucagon and GLP-1 receptors (Huypens et al., 2000). It was observed that glucose-stimulated insulin secretion from human islets was reduced by more than half in the presence of a glucagon receptor antagonist. The glucagon receptor antagonist also suppressed the potentiation of glucose-induced insulin release in response to exogenously added glucagon. These data suggest that expression of the glucagon or GLP-1 receptors may facilitate normal glucose-stimulated insulin secretion (Huypens et al., 2000).

### SUMMARY OF THE INVENTION

Thus, in accordance with the present invention, there is provided an isolated and purified polynucleotide encoding a polypeptide encoed by SEQ ID NOS:1-7, 66, 67, 69, 70, 72 or 74. More specifically, there is provided a cDNA sequence is selected from SEQ ID NOS:1-7, 66, 67, 69, 70, 72 or 74, or the complement thereof.

In addition, the present invention provides the preceding polynucleotides in an expression cassette comprising a promoter, wherein the polynucleotide is positioned so as to be under the regulatory control of the promoter. Examples of useful promoters include CMV IE, SV40 IE, RSV, GAPHD, or RIP1. The expression cassette may further comprise a polyadenylation signal, a heterologous leader sequence, an internal ribosome entry site and/or an origin of replication (bacterial or viral). The cassette may be included in a vector, for example, those derived from a bacterial plasmid or a viral genome, for example, an adenoviral vector, a retroviral vector, a vaccinia viral vector, a herpesviral vector, an adeno-associated viral vector, a polyoma viral vector. Also included may be a negative- or positive-selectable marker gene,

In another embodiment, there is provided an oligonucleotide of 15 to about 50 bases, comprising 15 or more consecutive bases of SEQ ID NOS:1-7, 66, 67, 69, 70, 72 or 74, or the complement thereof. The oligonucleotide may be 15, 16, 17, 18, 19, 20, 21, 22, 23, 24, 25, 30, 35, 40, 45 or 50 bases in length, and comprise 15, 16, 17, 18, 19, 20, 21, 22, 23, 24, 25, 30, 35, 40, 45 or 50 consecutive bases of SEQ ID NOS:1-7, or the complement thereof. In certain embodiment, the entire oligonucleotide base pairs with a sequence of SEQ ID NOS:1-7, 66, 67, 69, 70, 72 or 74, or the complement thereof. The oligonucleotide may further comprise a restriction enzyme site.

In yet another embodiment, there is provided a cell comprising a heterologous polynucleotide encoding a polypeptide having an amino acid sequence encoded by SEQ ID NOS:1-7, 66, 67, 69, 70, 72 or 74. The host cell may be a eukaryotic or a prokaryotic cell. The polynucleotide may further comprise a promoter active in the cell, the promoter positioned so as to provide regulatory control For the region encoding the polypeptide. The cell may further comprising a positive- and/or negative-selectable marker gene.

In still yet another embodiment, there is provided a method of expressing a polypeptide comprising (a) providing a host cell, the host cell comprising a heterologous an expression cassette comprising (a) a polynucleotide encoding a polypeptide having an amino acid sequence encoded by a polynucleotide selected from SEQ ID NOS:1-7, 66, 67, 69, 70, 72 or 74 and (b) a promoter active in the host cell, wherein the polynucleotide is positioned so as to be under the regulatory control of the promoter; and (b) culturing the host cell under conditions supporting the expression of the polypeptide. The host cell may be a prokaryotic or a eukaryotic host cell. The method may further comprise isolating the polypeptide from the host cell. The promoter may be an inducible promoter, and the conditions may further comprise induction of the promoter.

In an additional embodiment, there is provided a polypeptide having an amino acid sequence encoded by a polynucleotide selected from the group of SEQ ID NOS:1-7, 66, 67, 69, 70, 72 or 74. The polypeptide may be fused to a heterologous peptide or polypeptide sequence. Also contemplated is a peptide of 5 to about 35 residues comprising 5 or more consecutive residues of a polypeptide encoded by SEQ ID NOS:1-7, 66, 67, 69, 70, 72 or 74. The peptide may be 5, 6, 7, 8, 9, 10, 11, 12, 13, 14, 15, 16, 17, 18, 19, 20, 21, 22 23, 24, 25, 26, 27, 28, 29, 30, 31, 32, 33 34 or 35 residues in length, and may comprise 5, 6, 7, 8, 9, 10, 11, 12, 13, 14, 15, 16, 17, 18, 19, 20, 21, 22 23, 24, 25, 26, 27, 28, 29, 30, 31, 32, 33 34 or 35 consecutive residues of a polypeptide encoded by SEQ ID NOS:1-7, 66, 67, 69, 70, 72 or 74. Optionally, the peptide may be linked to a carrier molecule.

In still yet another embodiment, there is provided a monoclonal antibody that binds immunologically to a polypeptide having an amino acid sequence encoded by SEQ ID NOS:1-7, 66, 67, 69, 70, 72 or 74. The monoclonal antibody may be a single-chain monoclonal antibody. Also contemplated is a nucleic acid encoding such a single chain monoclonal antibody. The present invention also provides a polyclonal antibody composition, antibodies of which bind immunologically to a polypeptide having an amino acid sequence encoded by SEQ ID NOS:1-7, 66, 67, 69, 70, 72 or 74.

In yet an additional other embodiment, there is provided a method for modulating the glucose-responsiveness of a cell comprising introducing into the cell an expression cassette comprising (a) a polynucleotide encoding a polypeptide having an amino acid sequence encoded by SEQ ID NOS:1-7, 66, 67, 69, 70, 72 or 74 and (b) a promoter active in the cell, wherein the polynucleotide is positioned so as to be under the regulatory control of the promoter. The cell may be an insulin-producing cell, for example, a rat or a human cell. The cell may further comprise a positive- and/or negative-selectable marker gene, and the cell is immortalized (e.g., and insulinoma cell). The cell may further comprise one or more heterologous coding regions for insulin, GLUT-2, glucokinase, insulin-like growth factor GLP-1, IPF1, PC2, PC3, PAM, glucagon-like peptide I receptor, glucose-dependent insulinotropic polypeptide receptor, KIR, SUR, GHRFR, glucagon receptor, NKX2.2, NKX6.1 or GHRHR. The promoter may be an inducible and the method may further comprises induction. The cell may further comprise a positive- and/or negative-selectable marker gene. The method may further comprise introducing the cell into a mammalian host organism, for example, where the cell is encapsulated in a biocompatible device. The biocompatible device may further comprise a permeable membrane. Alternatively, one may down-regulate the expression of SEQ ID NOS:1-7, 66, 67, 69, 70, 72 or 74 using methods described in the following pages.

Also provided are:

a method of screening for a modulator of a polypeptide encoded by SEQ ID NOS:1-7, 66, 67, 69, 70, 72 or 74 comprising (a) providing a cell that expresses a polypeptide encoded by SEQ ID NOS:1-7, 66, 67, 69, 70, 72 or 74; (b) treating the cell with a candidate modulator; (c) determining the effect on expression of the polypeptide, as compared to a similar cell not treated with the candidate modulator, wherein a difference between the expression in treated and untreated cells indicates that the candidate modulator is a modulator of expression of a polypeptide encoded by SEQ ID NOS:1-7, 66, 67, 69, 70, 72 or 74; and,

a method for measuring the expression of a polypeptide encoded by SEQ ID NOS:1-7, 66, 67, 69, 70, 72 or 74 in a cell comprising (a) obtaining an mRNA population from the cell; (b) contacting the population with a DNA probe that hybridizes to SEQ ID NOS:1-7, 66, 67, 69, 70, 72 or 74, or the complement thereof; and (c) measuring hybridization of the probe to members of the population, whereby measuring hybridization of the probe measures the expression of a polypeptide encoded by SEQ ID NOS:1-7, 66, 67, 69, 70, 72 or 74; optionally including the step of amplification of members of the population.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Screen for glucose responsive INS-1-derived clones. Parental INS-1 cells were stably transfected with a plasmid containing the human proinsulin gene and the neomycin (G418) resistance gene. Following selection with G418, individual colonies were isolated and expanded, and screened by measuring the -fold increase in insulin secretion at 15 compared to 3 mM glucose. Data for 58 individual clones are presented, and represent the mean of three independent measurements per clone.

FIG. 2—Glucose stimulated insulin secretion by various INS-1 clones. Insulin secretion was measured by growth of cell lines 832/13 (13), 833/15 (15), 832/1 (1), 832/2 (2), 834/105 (105), and 834/112 (112) (Hohmeier et al., 2000; Chen et al., 2000). Cells were seeded in 12 well tissue culture plates, grown to confluence, and incubated in the presence of 1 mM glucose (open bars) or 20 mM glucose (filled bars) for 2 h. Media samples were collected and used for radioimmunoassay of insulin. Data are expressed as μU/ml. The abbreviated INS-1 clone numbers are shown at the bottom of the graph.

FIG. 3—Candidate genes analyzed in INS-1 clones. The figure lists candidate genes whose expression was compared in glucose responsive INS-1 derived cell lines (lines 832/13 and 833/15) versus poorly responsive lines (lines 832/1, 832/2, 834/105, 834/112) via a multiplex RT-PCR method (Gasa et al., 2000). Genes are grouped according to function: hormones, PTG (proteins targeting to glycoge), enzymes and transporters, and transcription factors. Among these candidate genes, only glucagon, and the transcription factors NKX 6.1 and NKX 2.2 were found to be expressed at significantly different levels in robustly glucose responsive compared to poorly glucose responsive clones.

FIG. 4—Glucagon expression in INS-1 clones. Using multiplex RT-PCR (MPX-PCR), expression of glucagon is measured in various INS-1 clones (numbers indicated at bottom of each graph; abbreviations as described in the legend to FIG. 2). MPX-PCR consisted of 18 cycles, and RT-PCR products were labeled with ³³P-dCTP. The left panel shows a representative autoradiogram of a polyacrylamide gel containing radiolabeled products generated by glucagon-specific oligonucleotide primers (bands labeled “glucagon”), relative to products generated with oligonucleotide sequences specific to tubulin (bands labeled “tubulin”). In the right hand panel, quantitative analysis of the ratio of glucagon:tubulin signals was performed by exposing gels to a phosphoimager screen, and processing the resulting scan with ImageQuant.

FIG. 5—Nkx2.2 & Nkx6.1 expression in INS-1 clones. Using multiplex RT-PCR (MPX-PCR), expression of Nkx 2.2 (left panel) and Nkx 6.1 (right panel) was evaluated in relation to an internal standard, glucose-6-phosphate dehydrogenase (G6PDH) in various INS-1 clones (numbers indicated at bottom of each graph; abbreviations as described in the legend to FIG. 2). Quantitative analysis of the ratio of Nkx 2.2/G6PDH (left panel) and Nkx 6.1/G6PDH (right panel) signals was performed by exposing gels to a phosphoimager screen, and processing the resulting scan with ImageQuant.

FIG. 6—Representational Difference Analysis (RDA). Schematic diagram of the representational difference analysis (RDA) method. Tester contains the gene of interest; drive is the substraction partner.

FIG. 7—Number of genes identified using RDA. Tabular summary of genes identified by RDA when INS-1 derived clone 832/13 was used as tester and clone 834/105 was used as driver (Strongly Responsive), or when clone 834/105 was used as tester and clone 832/13 was used as driver (Poorly Responsive). Genes identified in this fashion are classified as to whether they encode proteins of known function, are identical to expressed sequence tags encoding proteins of unknown function (ESTs), or are novel genes not present in any data bases available to the inventors (unknown).

FIG. 8—Genes identified by RDA. Tabular summary of genes identified by RDA comparison when INS-1 derived clone 832/13 was used as tester and clone 834/105 was used as driver (sequences specific for good responsders), or when clone 834/105 was used as tester and clone 832/13 was used as driver (sequences specific for poor responsers). In cases where the sequence of the gene identified by RDA matches a gene encoding a known protein, the protein is identified and a data base accession number is provided. In cases where the sequence corresponds to an expressed sequence tag (EST), this is noted and an accession number is provided. Genes identified by RDA with no match in the data base are annotated as “unknown, no homologies to ESTs”.

FIG. 9 and FIG. 10.—Expression pattern of genes identified using RDA. Using multiplex RT-PCR (MPX-PCR), the expression of genes isolated by RDA was measured in the various cell lines (cell line numbers indicated at top of each graph; abbreviations as described in the legend to FIG. 2), normalized to an internal standard, elongation factor i-α (EF1a). The left panel of FIG. 9 shows the expression of genes identified by RDA as preferentially expressed in robustly glucose responsive INS-1-derived clones, the right panel of FIG. 9 shows the expression of genes identified by RDA as preferentially expressed in poorly glucose responsive clones (right panel), and FIG. 10 shows the expression of an additional group of genes identified by RDA as preferentially expressed in robustly glucose responsive INS-1-derived clones.

FIG. 11 and FIG. 12—Tissue distribution of unknown genes and EST's. The tissue distribution pattern of genes identified by RDA that match ESTs or that have no match in the data base (unknowns) was measured by multiplex RT-PCR in the tissue types indicated at the bottom of each panel. Oligonucleotides specific for TATA box binding protein (TBP) were included in each multiplex PCR reaction as an internal standard.

### DESCRIPTION OF ILLUSTRATIVE EMBODIMENTS

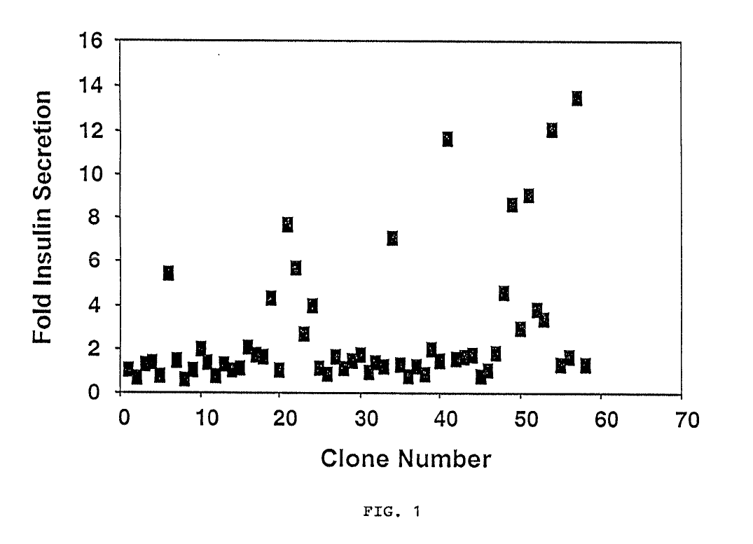

As discussed above, insulinoma cell lines are of primary interest in the development of insulin secreting host cells. One such cell line, the INS-1 line, was originally derived from a transplantable rat insulinoma tumor by Asfari et al. (1992). The cell line was genetically engineered to express the human insulin gene (Hohmeier et al., 2000; Chen et al., 2000). The clones derived from the human insulin engineering were heterogeneous with regard to magnitude of insulin secretion in response to glucose stimulation. Thus, 67% of the clones were found to be poorly responsive to glucose in terms of insulin secretion (≦2-fold stimulation by 15 mM compared to 3 mM glucose), 17% of the clones were moderately responsive (2-5-fold stimulation), and 16% were strongly responsive (5-13-fold stimulation) (FIG. 1). Since the cell lines had roughly equivalent insulin content, it was hypothesized that the differences in glucose stimulated insulin secretion were due to differentially expressed genes.

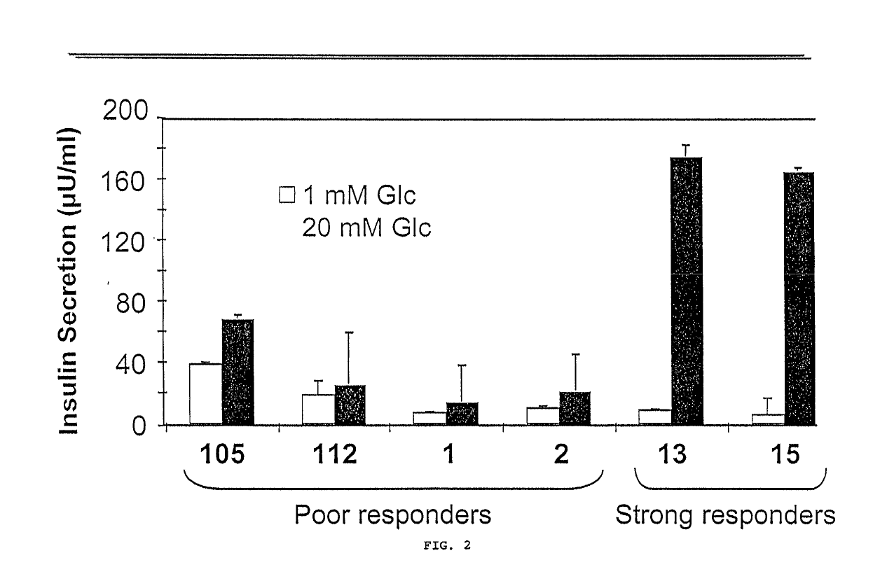

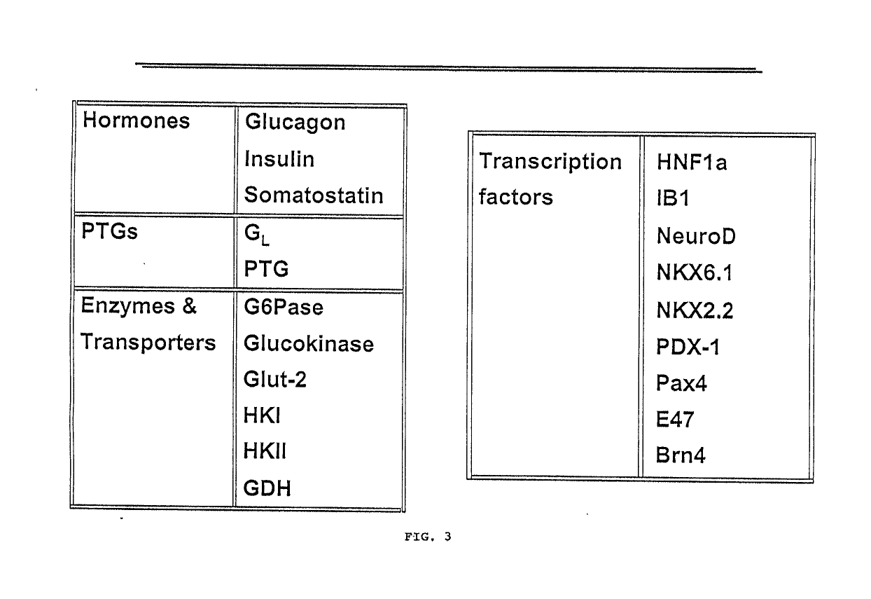

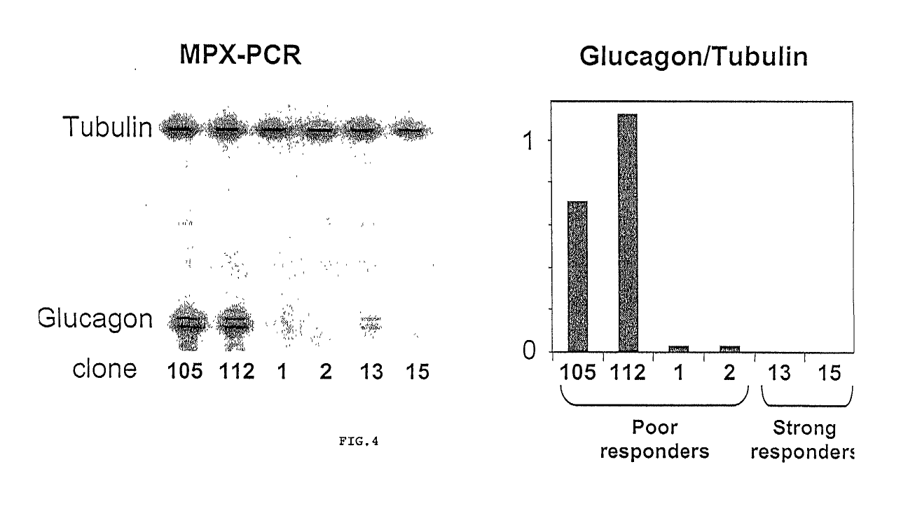

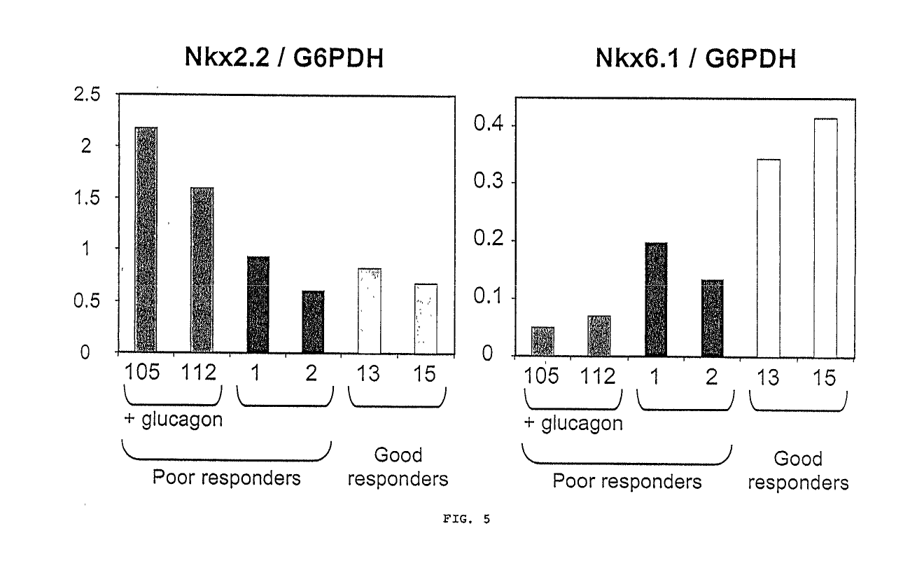

Two representative clones exhibiting robust glucose responsiveness (832/13 & 833/15) and four clones with poor glucose responsiveness (832/1, 832/2, 834/105 & 834/112) (FIG. 2) were screened for candidate genes known to play a role in insulin secretion using a multiplex RT-PCR method (Gasa et al., 2000). The panel of genes tested by this “candidate genes” approach is shown in FIG. 3. Of the 20 genes tested, 3 were found to be differentially expressed, as shown in FIGS. 4 and 5. Glucagon was found to be expressed in 2 of the poorly glucose responsive clones (834/105, 834/112), and not in the other poorly responsive clones (832/1, 832/2), or robustly responsive clones (832/13, 833/15) (FIG. 4). The transcription factor Nkx 2.2 had a similar expression pattern (highest in the glucagon positive, poorly responsive clones). In contrast, the transcription factor Nkx 6.1 was expressed at highest level in the robustly glucose responsive clones (832/13, 833/15), and at lower levels in the moderately or poorly responsive lines (FIG. 5). Stable expression of Nkx 6.1 in the poorly responsive clones did not convert these cells to a glucose-responsive phenotype. However, expression of Nkx 6.1 in clones 834/112 or 834/105 did reduce their glucagon expression, suggesting that Nkx 6.1 may be one gene involved in determining differentiated beta-cell phenotype in INS-1 derived cell lines and other insulinoma or neuroendocrine cells.

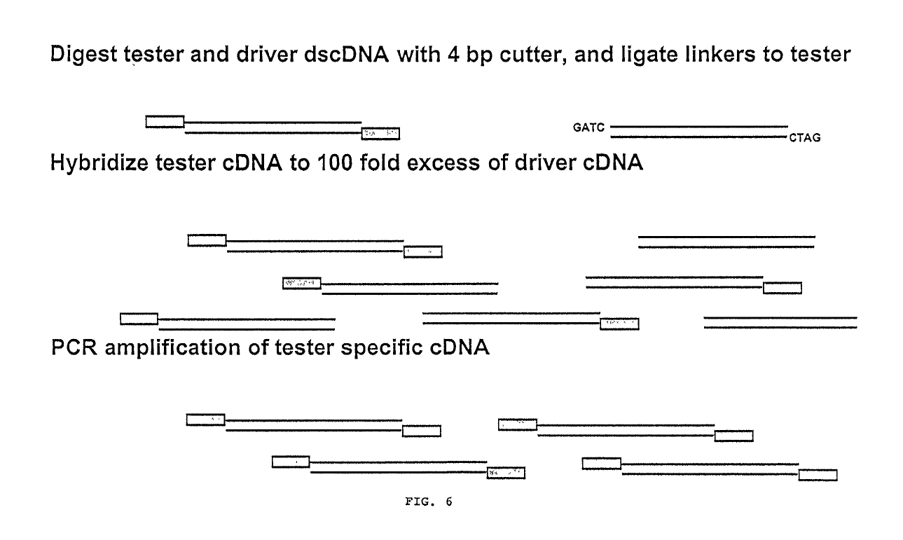

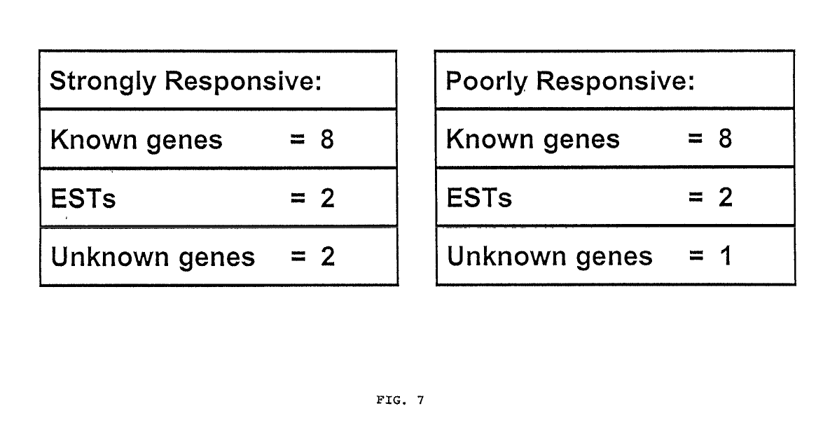

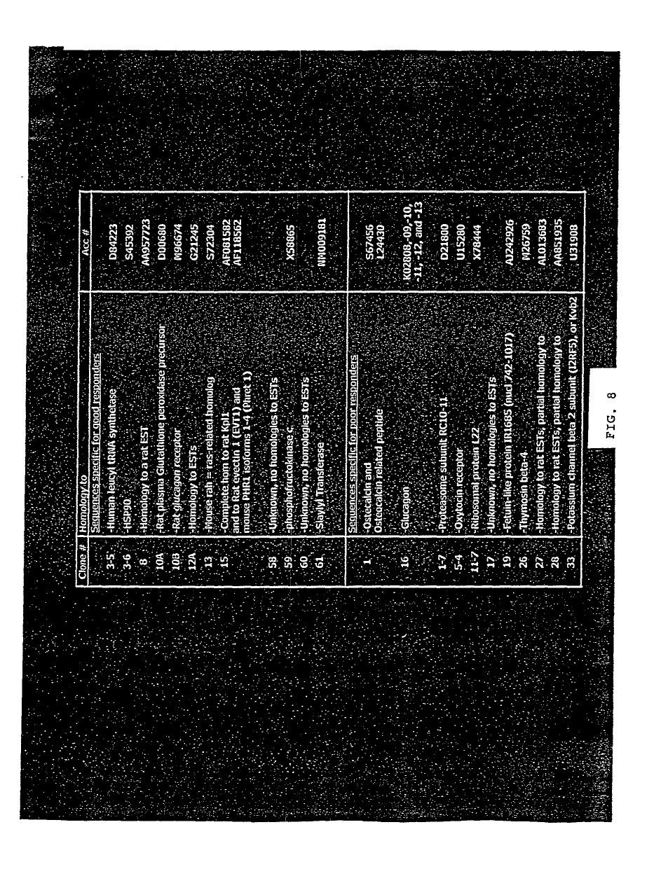

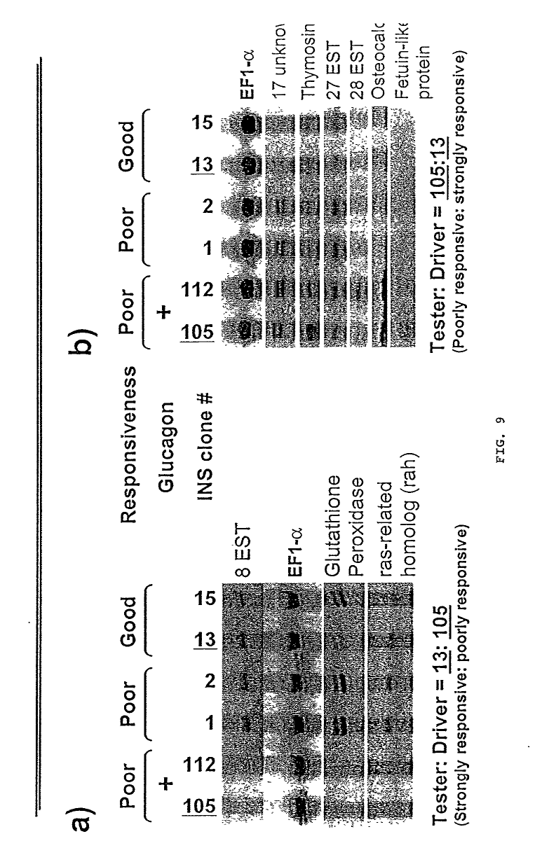

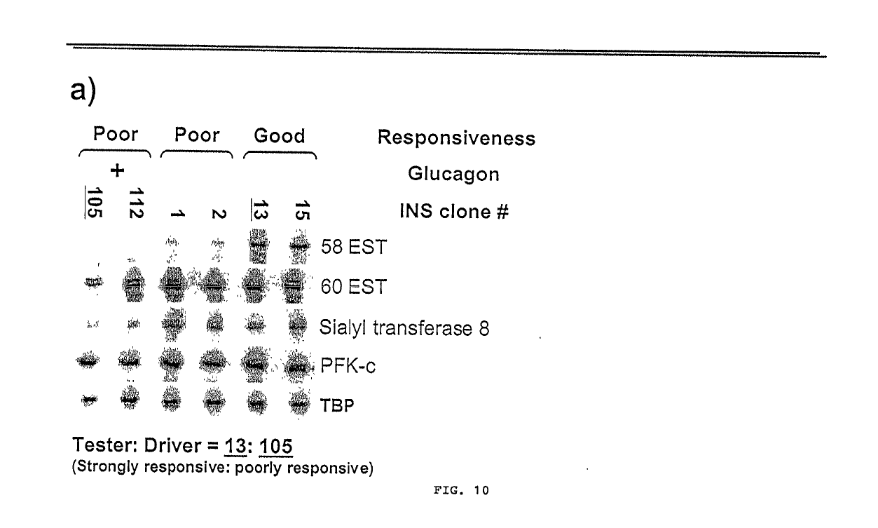

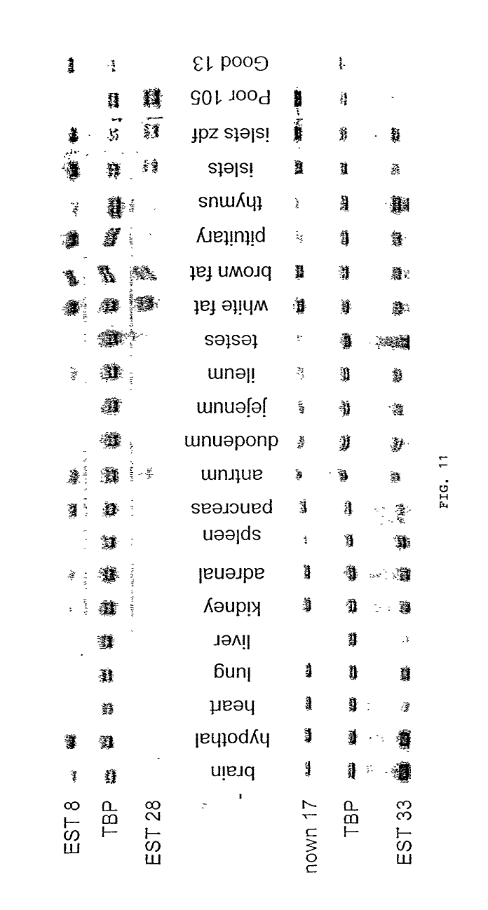

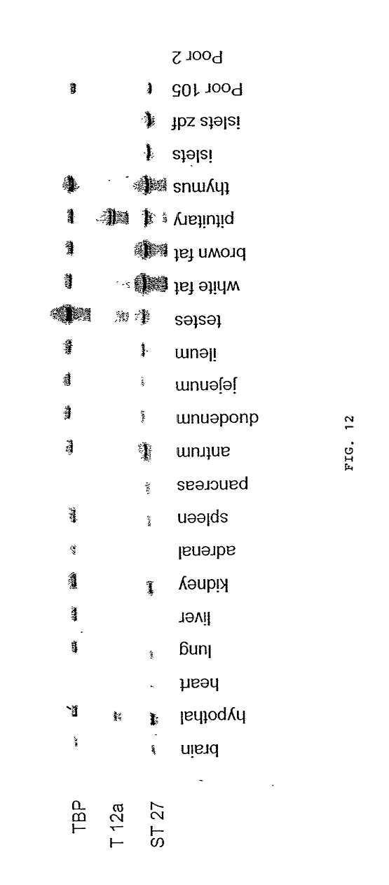

The inventors then focused on 2 clones, 832/13 (high responder) and 834/105 (low responder), and used a subtractive hybridization strategy called representational difference analysis (RDA) (Hubank & Schatz, 1994) to identify differentially expressed genes (for schematic summary of method, see FIG. 6). As summarized in FIG. 7, the inventors identified 11 genes preferentially expressed in the low responder clone (834/105) of which 8 were known genes, 2 matched sequences encoding expressed sequence tags (EST) of unknown function, and 1 was a novel, previously unreported sequence. The inventors also identified 12 genes preferentially expressed in the high responder clone (834/13). Of these 8 were known genes, 2 were ESTs of unknown function, and 2 were novel sequences. A listing of the genes uncovered in the RDA analysis is provided in FIG. 8. Importantly, the differential expression pattern of these genes has been confirmed by multiplex RT-PCR analysis (FIGS. 9 and 10). Furthermore, an analysis of the tissue expression pattern of these genes revealed that some are preferentially expressed in islets and other rare neuroendocrine tissues in the body, and are of relatively low abundance in visceral organs such as liver, muscle, kidney, etc. (FIGS. 11 and 12). A BLAST search reveals that the 5′ sequence of clone 8 (SEQ ID NO:66) shares high homology with mouse Ocat cDNA (bp 1933 to 2610 of SEQ ID NO: 68), while the 3′ sequence of clone 8 (SEQ ID NO:67) also shares high homology with mouse Ocat (bp 3119 to 3535 of SEQ ID NO:68). Szeto et al. (2000). A BLAST search of the human genome also identified the human homolog of clone 12a (SEQ ID NOS:69 and 70), which is shown as SEQ ID NO: 71 (genomic), SEQ ID NO: 72 (cDNA; ORF 28-1914) and SEQ ID NO:73 (protein). A rat/human 12a chimera is provided for cDNA (SEQ ID NO: 74; bp 1054-1257 being human) and protein (SEQ ID NO:75; residues 343-410 being human). The exon positions for each sequence are shown below:

| Exon # | SEQ ID NO: 71 | SEQ ID NO: 72 |
|---|---|---|
| 1 | 1-288 | 1-285 |
| 2 | 1529-1592 | 286-348 |
| 3 | 2095-2324 | 349-579 |
| 4 | 2576-2768 | 581-772 |
| 5 | 3224-3430 | 773-978 |
| 6 | 3952-4074 | 979-1098 |
| 7 | 4283-4337 | 1099-1152 |
| 8 | 4357-4453 | 1153-1248 |
| 9 | 4690-4791 | 1249-1350 |
| 10 | 4923-5029 | 1351-1455 |
| 11 | 5421-5602 | 1456-1635 |
| 12 | 6992-8247 | 1636-2892 |

The human 12a protein sequence gives 630 amino acids with a predicted molecular weight of about 71 kDa. High homlogy exists at residues 232 to 403 with Rho-type GTPase Activating Proteins. The rat/human 12a chimera uses residues 349 to 434 of human to “splice” the amino- and carboxy-terminal portions of the rat 12a clones (residues 343 to 410). The predicted 627 residue protein has a predicted molecular weight of 71 kDa.

A. Peptides, Polypeptides and Antibodies In certain embodiments, the present invention concerns novel compositions comprising a polypeptide encoded by SEQ ID NOS:1-7, 66, 67, 69, 70, 72 or 74. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 5 to about 100 amino acids. All the “proteinaceous” terms described above may be used interchangeably herein.

In certain embodiments the size of the at least one proteinaceous molecule may comprise, but is not limited to about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino molecule residues, and any range derivable therein.

As used herein, an “amino acid molecule” refers to any amino acid, amino acid derivitive or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 1
Modified and Unusual Amino Acids

| Abbr. | Amino Acid |
|---|---|
| Aad | 2-Aminoadipic acid |
| Baad | 3-Aminoadipic acid |
| Bala | β-alanine, β-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| AIle | allo-Isoleucine |
| MeGly | N-Methylglycine, |
| | sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Organisms include, but are not limited to, Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

In certain embodiments, the proteinaceous composition may be used to elicit an one antibody. It is contemplated that antibodies that bind immunoogically to the specified polypeptides may be useful for protein purification and in diagnostic contexts. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single-chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Polyclonal antibodies generally are raised in animals by multiple subcutaneous (sc) or intraperitoneal (ip) injections of protein and an adjuvant. It may be useful to conjugate antigen or a fragment containing the target amino acid sequence to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glytaradehyde, succinic anhydride, etc.

Animals are immunized against the immunogenic conjugates or derivatives by combining 1 mg of 1 μg of conjugate (for rabbits or mice, respectively) with 3 volumes of Freud's complete adjuvant and injecting the solution intradermally at multiple sites. One month later the animals are boosted with ⅕ to 1/10 the original amount of conjugate in Freud's complete adjuvant by subcutaneous injection at multiple sites. Seven to 14 days later the animals are bled and the serum is assayed for antibody titer. Animals are boosted until the titer plateaus. Preferably, the animal boosted with the antigen, but conjugated to a different protein and/or through a different cross-linking reagent. Conjugates also can be made in recombinant cell culture as protein fusions. Also, aggregating agents such as alum are used to enhance the immune response.

Monoclonal antibodies are obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies.

For example, the monoclonal antibodies of the invention may be made using the hybridoma method first described by Kohler & Milstein (1975), or may be made by recombinant DNA methods. U.S. Pat. No. 4,816,567.

In the hybridoma method, a mouse or other appropriate host animal, such as hamster is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell. Goding, Monoclonal Antibodies: Principles and Practice, pp.59-103 (Academic Press, 1986).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high level expression of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Culture medium in which hybridoma cells are growing is assayed for production of monoclonal antibodies. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by the Scatchard analysis of Munson & Pollard (1980).

After hybridoma cells are identified that produce antibodies of the desired specificity, affinity, and/or activity, the clones may be subcloned by limiting dilution procedures and grown by standard methods. Goding, Monoclonal Antibodies: Principles and Practice, pp.59-104 (Academic Press, 1986). Suitable culture media for this purpose include, for example, Dulbecco's Modified Eagle's Medium or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morriso et al., Proc. Nat'l Acad. Sci. USA 81, 6851 (1984), or by covalently joining to the immunoglobulin coding sequence all or part of the coding sequence for a non-immunoglobulin polypeptide. In that manner, “chimeric” or “hybrid” antibodies are prepared.

Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for one antigen and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

For diagnostic applications, the antibodies of the invention typically will be labeled with a detectable moiety. The detectable moiety can be any one which is capable of producing, either directly or indirectly, a detectable signal. For example, the detectable moiety may be a radioisotope, a fluorescent or chemiluminescent compound, such as fluorescein isothiocyanate, rhodamine, or luciferin; biotin; or an enzyme, such as alkaline phosphatase, beta-galactosidase or horseradish peroxidase.

Any method known in the art for separately conjugating the antibody to the detectable moiety may be employed, including those methods described by Hunter et al., Nature 144:945 (1962); David et al., Biochemistry 13:1014 (1974); Pain et al., J Immunol. Meth. 40:219 (1981); and Nygren, Histochem. and Cytochem. 30:407 (1982).

The antibodies of the present invention may be employed in any known assay method, such as competitive binding assays, direct and indirect sandwich assays, and immunoprecipitation assays. Zola, Monoclonal Antibodies: A Manual of Techniques, pp.147-158 (CRC Press, Inc., 1987).

Competitive binding assays rely on the ability of a labeled standard to compete with the test sample analyte for binding with a limited amount of antibody. The amount of analyte in the test sample is inversely proportional to the amount of standard that becomes bound to the antibodies. To facilitate determining the amount of standard that becomes bound, the antibodies generally are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three part complex. U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

B. Nucleic Acids

Certain embodiments of the present invention concerns the nucleic acids of SEQ ID NOS:1-7, 66, 67, 69, 70, 72 or 74. In certain aspects, both wild-type and mutant versions of these sequenes are employed. In particular aspects, a nucleic acid encodes for or comprises a transcribed nucleic acid. In other aspects, a nucleic acid comprises a nucleic acid segment of SEQ ID NOS:1-7, 66, 67, 69, 70, 72 or 74, or a biologically functional equivalent thereof.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 8 and about 100 nucleobases in length. The term “polynucleotide” refers to at least one molecule of greater than about 100 nucleobases in length.

Herein certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

“Isolated substantially away from other coding sequences” means that the gene of interest forms the significant part of the coding region of the nucleic acid, or that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

(i) Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A table non-limiting, purine and pyrimidine derivatives and analogs is also provided herein below.

TABLE 2
Purine and Pyrmidine Derivatives or Analogs

| Abbr. | Modified base description |
|---|---|
| ac4c | 4-acetylcytidine |
| Chm5u | 5-(carboxyhydroxylmethyl)uridine |
| Cm | 2′-O-methylcytidine |
| Cmnm5s2u | 5-carboxymethylamino-methyl-2-thioridine |
| Cmnm5u | 5-carboxymethylaminomethyluridine |
| D | Dihydrouridine |
| Fm | 2′-O-methylpseudouridine |
| Gal q | Beta,D-galactosylqueosine |
| Gm | 2′-O-methylguanosine |
| I | Inosine |
| I6a | N6-isopentenyladenosine |
| m1a | 1-methyladenosine |
| m1f | 1-methylpseudouridine |
| m1g | 1-methylguanosine |
| m1I | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| Mam5u | 5-methylaminomethyluridine |
| Mam5s2u | 5-methoxyaminomethyl-2-thiouridine |
| Man q | Beta,D-mannosylqueosine |
| Mcm5s2u | 5-methoxycarbonylmethyl-2-thiouridine |
| Mcm5u | 5-methoxycarbonylmethyluridine |
| Mo5u | 5-methoxyuridine |
| Ms2i6a | 2-methylthio-N6-isopentenyladenosine |
| Ms2t6a | N-((9-beta-D-ribofuranosyl-2-methylthiopurine-6-yl) |
| | carbamoyl)th |
| Mt6a | N-((9-beta-D-ribofuranosylpurine-6-yl)N-methyl- |
| | carbamoyl)threo |
| Mv | Uridine-5-oxyacetic acid methylester |
| o5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6-yl)carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, (acp3)u |

A nucleobase may be comprised in a nucleside or nucleotide, using any chemical or natural synthesis method described herein or known to one of ordinary skill in the art.

(ii) Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

(iii) Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

(iv) Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as flourescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moeity which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moeity replacing phosphodiester backbone moeity used for improved nuclease resistance, cellular uptake and regulating RNA expression; U.S. Pat. No. 5,858,988 which describes hydrophobic carrier agent attached to the 2′-O position of oligonuceotides to enhanced their membrane permeability and stability; U.S. Pat. No. 5,214,136 which describes olignucleotides conjugaged to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid.

(v) Polyether and Peptide Nucleic Acids

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moeity that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit nucleic acid amplification, such as in PCR, to reduce false positives and discriminate between single base mutants, as described in U.S. Pat. No. 5,891,625. Other modifications and uses of nucleic acid analogs are known in the art. In a non-limiting example, U.S. Pat. No. 5,786,461 describes PNAs with amino acid side chains attached to the PNA backbone to enhance solubility of the molecule. Another example is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

(vi). Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266 032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which are incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

(vii) Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

(viii) Nucleic Acid Segments

In certain embodiments, the nucleic acid is a nucleic acid segment. As used herein, the term “nucleic acid segment,” are smaller fragments of a nucleic acid, such as for non-limiting example, those that encode only part of the SEQ ID NOS:1-7, 66, 67, 69, 70, 72 or 74. Thus, a “nucleic acid segment” may comprise any part of a gene sequence, of from about 8 nucleotides to the full length of the SEQ ID NOS:1-7, 66, 67, 69, 70, 72 or 74.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n +y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. This algorithm would be applied to each of SEQ ID NOS:1-7, 66, 67, 69, 70, 72 or 74. As used herein, a “probe” generally refers to a nucleic acid used in a detection method or composition. As used herein, a “primer” generally refers to a nucleic acid used in an extension or amplification method or composition.

In a non-limiting example, one or more nucleic acid constructs may be prepared that include a contiguous stretch of nucleotides identical to or complementary to SEQ ID NOS:1-7, 66, 67, 69, 70, 72 or 74. A nucleic acid construct may be about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 60, about 70, about 80, about 90, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 14, about 15, about 16, about 17, about 18, about 19, about, 20, about 21, about 22, about 23, about 24, about 25, about 26, about 27, about 28, about 29, about 30, about 35, about 40, about 50, about 60, about 70, about 80, abot 90, about 100, about 125, about 150, about 175, about 200, about 500, about 1,000, about 10,000, about 50,000, about 100,000, about 250,00, about 500,00, about 1,000,000 or more bases.

(ix) Nucleic Acid Complements

The present invention also encompasses a nucleic acid that is complementary to a SEQ ID NOS:1-7, 66, 67, 69, 70, 72 or 74. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule of SEQ ID NOS:1-7, 66, 67, 69, 70, 72 or 74 during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

(x) Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

(xi) Genetic Degeneracy

The term “functionally equivalent codon” is used herein to refer to codons that encode the same amino acid, such as the six codons for arginine and serine, and also refers to codons that encode biologically equivalent amino acids. For optimization of expression in human cells, the codons are shown in Table 3 in preference of use from left to right. Thus, the most preferred codon for alanine is thus “GCC”, and the least is “GCG” (see Table 3 below). Codon usage for various organisms and organelles can be found at the website www.kazusa.orjp/codon/, incorporated herein by reference, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

TABLE 3
Preferred Human DNA Codons

| Amino Acids | | | Codons | | | | | |
|---|---|---|---|---|---|---|---|---|
| Alanine | Ala | A | GCC | GCT | GCA | GCG | | |
| Cysteine | Cys | C | TGC | TGT | | | | |
| Aspartic acid | Asp | D | GAC | GAT | | | | |
| Glutamic acid | Glu | E | GAG | GAA | | | | |
| Phenylalanine | Phe | F | TTC | TTT | | | | |
| Glycine | Gly | G | GGC | GGG | GGA | GGT | | |
| Histidine | His | H | CAC | CAT | | | | |
| Isoleucine | Ile | I | ATC | ATT | ATA | | | |
| Lysine | Lys | K | AAG | AAA | | | | |
| Leucine | Leu | L | CTG | CTC | TTG | CTT | CTA | TTA |
| Methionine | Met | M | ATG | | | | | |
| Asparagine | Asn | N | AAC | AAT | | | | |
| Proline | Pro | P | CCC | CCT | CCA | CCG | | |
| Glutamine | Gln | Q | CAG | CAA | | | | |
| Arginine | Arg | R | CGC | AGG | CGG | AGA | CGA | CGT |
| Serine | Ser | S | AGC | TCC | TCT | AGT | TCA | TCG |
| Threonine | Thr | T | ACC | ACA | ACT | ACG | | |
| Valine | Val | V | GTG | GTC | GTT | GTA | | |
| Tryptophan | Trp | W | TGG | | | | | |
| Tyrosine | Tyr | Y | TAC | TAT | | | | |

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

Excepting intronic and flanking regions, and allowing for the degeneracy of the genetic code, nucleic acid sequences that have between about 70% and about 79%; or more preferably, between about 80% and about 89%; or even more particularly, between about 90% and about 99%; of nucleotides that are identical to the nucleotides of SEQ ID NOS:1-7, 66, 67, 69, 70, 72 or 74 will be nucleic acid sequences that are “essentially as set forth in SEQ ID NOS:1-7, 66, 67, 69, 70, 72 or 74.”

C. Engineering Host Cells

Engineering of host cells will advantageously make use of many endogenous attributes of these cells. Regulated secretory cells present a natural bioreactor containing specialized enzymes involved in the processing and maturation of secreted proteins. All cells secrete proteins through a constitutive, non-regulated secretory pathway. A subset of cells are able to secrete proteins through a specialized regulated secretory pathway. Proteins destined for secretion by either mechanism are targeted to the endoplasmic reticulum and pass through the golgi apparatus. Constitutively secreted proteins pass directly from the golgi to the plasma membrane in vesicles, fusing and releasing the contents constitutively without the need for external stimuli. In cells with a regulated pathway, proteins leave the golgi and concentrate in storage vesicles or secretory granules. Release of the proteins from secretory granules is regulated, requiring an external stimuli. This external stimuli, defined as a secretagogue, can vary depending on cell type, optimal concentration of secretagogue, and dynamics of secretion. Proteins can be stored in secretory granules in their final processed form for long periods of time. In this way a large intracellular pool of mature secretory product exists which can be released quickly upon secretagogue stimulation.

A cell specialized for secreting proteins via a regulated pathway can also secrete proteins via the constitutive secretory pathway. Many cell types secrete proteins by the constitutive pathway with little or no secretion through a regulated pathway. As used herein, “secretory cell” defines cells specialized for regulated secretion, and excludes cells that are not specialized for regulated secretion. The regulated secretory pathway is found in secretory cell types such as endocrine, exocrine, neuronal, some gastrointestinal tract cells and other cells of the diffuse endocrine system. Nonetheless, modifications may be needed in order to fully exploit a cell's ability to deliver a heterologous polypeptide in an in vivo context. The following sections will discuss some of these modifications.

(i) Modulation of Glucose Responsive Genes

In accordance with the present invention, the inventors propose to modulate the expression of the polypeptides encoded by SEQ ID NOS:1-7, 66, 67, 69, 70, 72 or 74. For SEQ ID NOS:1-7, 66, 67, 69, 70, 72 or 74, the primary application will be the introduction of one or more nucleic acids encoding corresponding polypeptides into a host cell, where the expression of the polypeptide is driven by a promoter active in the host cell. Methods for engineering cells for high level expression, and for expression of ancillary polypeptide expression, are discussed elsewhere in this document. Also discussed elsewhere are methods for propagating such cells, introducing them into suitable devices, and implanting the devices in a subject.

(ii) Suitable Starting Cells for Engineering

According to the present invention, a wide variety of host cells may be used for genetic engineering. In addition to possessing secretory capabilities, other desireable properties include ease of culture, genetic stability during successive rounds of culture, multi-passage capability, resistance to suboptimal culture condition, insulin secretion, readily manipulable from a genetic engineering standpoint, non-toxic, good survival and function in transplantable device, capable of large scale production, post-translational peptide processing, high peptide production capabilities, cytokine resistance (U.S. Pat. No. 6,171,856), and/or non-tumorigenic. Of particular interest are insulinoma cell lines such as those deriving from the INS-1 line described by Asafari et al. (1992), e.g., those discussed by Hobmeimer et al. (2000).

Suitable cells also include any of the cells mentioned in U.S. Pat. Nos. 5,427,940, 5,744,327, 5,747,325, 5,792,656, 5,993,799, 5,811,266, 6,087,129, 6,110,707, and 6,171,856.

(iii) Methods for Blocking Endogenous Protein Production

Blocking expression of an endogenous gene product is another modification of host cells that may prove useful. A number of basic approaches are contemplated for blocking of expression of an endogenous gene in host cells. First, constructs are designed to homologously recombine into particular endogenous gene loci, rendering the endogenous gene nonfunctional. Second, constructs are designed to randomly integrate throughout the genome, resulting in loss of expression of the endogenous gene. Third, constructs are designed to introduce nucleic acids complementary to a target endogenous gene. Expression of RNAs corresponding to these complementary nucleic acids will interfere with the transcription and/or translation of the target sequences. Fourth, constructs are designed to introduce nucleic acids encoding ribozymes—RNA-cleaving enzymes—that will specifically cleave a target mRNA corresponding to the endogenous gene. Fifth, endogenous gene can be rendered dysfunctional by genomic site directed mutagenesis

Antisense. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

Ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990; Sioud et al., 1992). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

Homologous Recombination. Another approach for blocking of endogenous protein production involves the use of homologous recombination. Homologous recombination relies, like antisense, on the tendency of nucleic acids to base pair with complementary sequences. In this instance, the base pairing serves to facilitate the interaction of two separate nucleic acid molecules so that strand breakage and repair can take place. In other words, the “homologous” aspect of the method relies on sequence homology to bring two complementary sequences into close proximity, while the “recombination” aspect provides for one complementary sequence to replace the other by virtue of the breaking of certain bonds and the formation of others.

Put into practice, homologous recombination is used as follows. First, a target gene is selected within the host cell. Sequences homologous to the target gene are then included in a genetic construct, along with some mutation that will render the target gene inactive (stop codon, interruption, etc.). The homologous sequences flanking the inactivating mutation are said to “flank” the mutation. Flanking, in this context, simply means that target homologous sequences are located both upstream (5′) and downstream (3′) of the mutation. These sequences should correspond to some sequences upstream and downstream of the target gene. The construct is then introduced into the cell, thus permitting recombination between the cellular sequences and the construct.

As a practical matter, the genetic construct will normally act as far more than a vehicle to interrupt the gene. For example, it is important to be able to select for recombinants and, therefore, it is common to include within the construct a selectable marker gene. This gene permits selection of cells that have integrated the construct into their genomic DNA by conferring resistance to various biostatic and biocidal drugs. In addition, a heterologous gene that is to be expressed in the cell also may advantageously be included within the construct. The arrangement might be as follows:

. . . vector•5′-flanking sequence•heterologous gene•selectable marker gene•flanking sequence-3′•vector . . .

Thus, using this kind of construct, it is possible, in a single recombinatorial event, to (i) “knock out” an endogenous gene, (ii) provide a selectable marker for identifying such an event and (iii) introduce a heterologous gene for expression.

Another refinement of the homologous recombination approach involves the use of a “negative” selectable marker. This marker, unlike the selectable marker, causes death of cells which express the marker. Thus, it is used to identify undesirable recombination events. When seeking to select homologous recombinants using a selectable marker, it is difficult in the initial screening step to identify proper homologous recombinants from recombinants generated from random, non-sequence specific events. These recombinants also may contain the selectable marker gene and may express the heterologous protein of interest, but will, in all likelihood, not have the desired “knock out” phenotype. By attaching a negative selectable marker to the construct, but outside of the flanking regions, one can select against many random recombination events that will incorporate the negative selectable marker. Homologous recombination should not introduce the negative selectable marker, as it is outside of the flanking sequences.

In a particular aspect of this embodiment, the negative selectable maker is GLUT-2. It is also contemplated that GLUT-5 would function in a similar manner to GLUT-2. Therefore, the selection protocols described are intended to refer to the use of both GLUT-2 and GLUT-5.

In a first embodiment, a target gene within a GLUT-2⁻ host cell is selected as the location into which a selected gene is to be transferred. Sequences homologous to the target gene are included in the expression vector, and the selected gene is inserted into the vector such that target gene homologous sequences are interrupted by the selected gene or, put another way, such the target gene homologous sequences “flank” the selected gene. In preferred embodiments, a drug selectable marker gene also is inserted into the target gene homologous sequences. Given this possibility, it should be apparent that the term “flank” is used broadly herein, namely, as describing target homologous sequences that are both upstream (5′) and downstream (3′) of the selected gene and/or the drug selectable marker gene. In effect, the flanking sequences need not directly abut the genes they “flank.”

The construct for use in this embodiment is further characterized as having a negative selectable marker gene attached thereto. Thus, one possible arrangement of sequences would be:

. . . 5′-negative selectable marker gene•flanking target sequences•selected gene•drug-selectable marker gene•flanking target sequences-3′. . .

Of course, the negative selectable marker could be placed at the 3′-end of the construct and the selected gene and drug-selectable marker genes could exchange positions.

On the other hand, site-specific recombination, relying on the homology between the vector and the target gene, will result in incorporation of the selected gene and the drug selectable marker gene only; negative selectable marker sequences will not be introduced in the homologous recombination event because they lie outside the flanking sequences. These cells will be drug resistant and but not acquire the negative selectable marker sequences and, thus, remain insensitive to negative drug selection. This double-selection procedure should yield recombinants that lack the target gene and express the selected gene. Further screens for these phenotypes, either functional or immunologic, may be applied.

A modification of this procedure is one where no selected gene is included, i.e., only the selectable marker is inserted into the target gene homologous sequences. Use of this kind of construct will result in the “knock-out” of the target gene only.

Genomic Site-Directed Mutagenesis with Oligonucleotides. Through analysis of radiation-sensitive mutants of Ustilago maydis, genes have been characterized that participate in DNA repair (Tsukuda et al., 1989; Bauchwitz and Holloman, 1990). One such gene, REC2, encodes a protein that catalyzes homologous pairing between complementary nucleic acids and is required for a functional recombinational repair pathway (Kmiec et al., 1994; Rubin et al., 1994). In vitro characterization of the REC2 protein showed that homologous pairing was more efficient between RNA-DNA hybrids than the corresponding DNA duplexes (Kmiec et al, 1994; PCT, WO 96/22364). However, efficiency in pairing between DNA:DNA duplexes could be enhanced by increasing the length of the DNA oligonucleotides (Kmiec et al., 1994). These observations led investigators to test the use of chimeric RNA-DNA oligonucleotides (RDOs) in the targeted modification of genes in mammalian cell lines (Yoon et al., 1996; Cole-Strauss et al., 1996; PCT WO95/15972). The RNA-DNA oligonucleotides that were used to test this application contained self-annealing sequences such that double-hairpin capped ends are formed. This feature is thought to increase the in vivo half-life of the RDO by decreasing degradation by helicases and exonucleases. Further, the RDOs contained a single base pair that differs from the target sequence and otherwise aligns in perfect register. It is believed that the single mismatch will be recognized the DNA repair enzymes. And the RDOs contained RNA residues modified by 2′-O-methylation of the ribose sugar. Such modification makes the RDO resistant to degradation by ribonuclease activity (Monia et al., 1993).

Two separate experimental systems have been used to test the use of RDOs for targeted gene disruption in mammalian cell lines. In one system RDOs were used to target and correct an alkaline phosphatase cDNA in that was maintained in the episomal DNA of Chinese hamster ovary cells. An inactive form of alkaline phosphatase was converted to a wild-type form with an efficiency of about 30% (Yoon et al., 1996). In a second system, a genetic mutation within chromosomal DNA was targeted and corrected. A lymphoid blast cell line was derived from a patient with sickle cell disease who was homozygous for a point mutation in the β-globin gene. Here again the overall frequency of gene conversion from the mutant to the wild-type form was very high and was found to be dose-dependent on the concentration of the RDOs (Cole-Strauss et al., 1996).

If the use of RDOs or DNA oligonucleotides for the purposes of targeted gene conversion is broadly applicable to various mammalian cell lines, then it offers several advantages to current technologies that have been used to accomplish gene disruption such as homologous recombination. First, if gene conversion by RDO or DNA oligonucleotides occurs in various cell lines at an efficiency of 30% then this will represent a much higher rate than has been reported for targeted gene disruption via homologous recombination. Secondly, only short sequences are required for gene disruption by RDOs or DNA oligonucleotides(typically 60 mers to 70 mers); whereas homologous recombination requires very long stretches of complementary sequences. Homologous sequences from 9 to 15 kilobases are typically recommended in the construction of targeting vectors. As a result, construction of DNA vectors for homologous recombination usually involves extensive gene mapping studies and time consuming efforts in the isolation of genomic DNA sequences. Such efforts are unnecessary if RDOs are used for targeted gene conversions. Thirdly, assays for gene conversion by RDOs can be performed 4 to 6 hours following introduction of the RDOs or DNA oligonucleotides into the cell. In contrast, gene conversion by homologous recombination requires a relatively long period of time (days to weeks) between the time of introducing the targeting vector DNA and assaying for recombinants.

Random Integration. Though lacking the specificity of homologous recombination, there may be situations where random integration will be used as a method of knocking out a particular endogenous gene. Unlike homologous recombination, the recombinatorial event here is completely random, i.e., not reliant upon base-pairing of complementary nucleic acid sequences. Random integration is like homologous recombination, however, in that a gene construct, often containing a heterologous gene and a selectable marker, integrates into the target cell genomic DNA via strand breakage and reformation.

Because of the lack of sequence specificity, the chances of any given recombinant integrating into the target gene are greatly reduced. Also possible is integration into a second loci, resulting in the loss of expression of the gene of interest. This second locus could encode a transcription factor needed for expression of the first gene, a locus control region needed for the expression of the first gene, etc. As a result, it may be necessary to “brute force” the selection process. In other words, it may be necessary to screen hundreds of thousands of drug-resistant recombinants before a desired mutant is found. Screening can be facilitated, for example, by examining recombinants for expression of the target gene using immunologic or even functional tests; expression of the target gene indicate recombination elsewhere and, thus, lack of suitability.

(iv) Methods for Increasing Production of Recombinant Polyeptides from Secretory Cells

The present invention also contemplates augmenting or increasing the capabilities of cells to produce certain polypeptides, in particular, the polypeptides encoded by SEQ ID NOS:1-7, 66, 67, 69, 70, 72 or 74. This can be accomplished, in some instances, by overexpressing the proteins involved in protein processing, such as the endoproteases PC2 and PC3 (Steiner et al., 1992) or the peptide amidating enzyme, PAM (Eipper et al., 1992a) in the case of amidated peptide hormones.

Proteins involved in Secretion. Expression of proteins involved in maintaining the specialized phenotype of host cells, especially their secretory capacity, is important. Engineering the overexpression of a cell type-specific transcription factor such as the Insulin Promoter Factor 1 (IPF1) found in pancreatic β-cells (Ohlsson et al., 1993) could increase or stabilize the capabilities of engineered neuroendocrine cells. Insulin promoter factor 1 (IPF-1; also referred to as STF-1, IDX-1, PDX-1 and βTF-1) is a homeodomain-containing transcription factor proposed to play an important role in both pancreatic development and insulin gene expression in mature β cells (Ohlsson et al., 1993, Leonard et al., 1993, Miller et al., 1994, Kruse et al., 1993). In embryos, IPF-1 is expressed prior to islet cell hormone gene expression and is restricted to positions within the primitive foregut where pancreas will later form. Indeed, mice in which the IPF-1 gene is disrupted by targeted knockout do not form a pancreas (Jonsson et al., 1994). Later in pancreatic development, as the different cell types of the pancreas start to emerge, IPF-1 expression becomes restricted predominantly to β cells. IPF-1 binds to TAAT consensus motifs contained within the FLAT E and P1 elements of the insulin enhancer/promoter, whereupon, it interacts with other transcription factors to activate insulin gene transcription (Peers et al., 1994).

Stable overexpression of IPF-1 in neuroendocrine β cell lines will serve two purposes. First, it will increase transgene expression under the control of the insulin enhancer/promoter. Second, because IPF-1 appears to be critically involved in β cell maturation, stable overexpression of IPF-1 in β cell lines should cause these mostly dedifferentiated β-cells to regain the more differentiated function of a normal animal β cell. If so, then these redifferentiated β cell lines could potentially function as a more effective neuroendocrine cell type for cell-based delivery of fully processed, bioactive peptide hormones.

Ancillary Glucose-Response Genes. Also, further engineering of cells to generate a more physiologically-relevant regulated secretory response is claimed. Examples would include engineering the ratios of glucokinase to hexokinase in rat insulinoma cells that also overexpress the Type II glucose transporter (GLUT-2) such that a physiologically-relevant glucose-stimulated secretion of peptide hormones is achieved. Other examples include engineering overexpression of other signaling proteins known to play a role in the regulated secretory response of neuroendocrine cells. These include cell surface proteins such as the β-cell-specific inwardly rectifying potassium channel (BIR; Inagaki et al., 1995), involved in release of the secretory granule contents upon glucose stimulation, the sulfonylurea receptor (SUR), and ATP sensitive channel. Other cell surface signaling receptors which help potentiate the glucose-stimulated degranulation of β-cells including the glucagon-like peptide I receptor (Thorens, 1992) and the glucose-dependent insulinotropic polypeptide receptor (also known as gastric inhibitory peptide receptor) (Usdin, 1993) can be engineered into neuroendocrine cells. These β-cell-specific signaling receptors, as well as GLUT-2 and glucokinase, are involved in secretory granule release in response to glucose. In this way, glucose stimulated release of any heterologous peptide targeted to the secretory granule can be engineered. Alternatively, other cell surface signaling proteins involved in non-glucose-stimulated release of secretory granule contents can be engineered into neuroendocrine cells. Examples would include releasing factor receptors such as Growth Hormone Releasing Factor Receptor (Lin et al., 1992) and Somatostatin or Growth Hormone Releasing Hormone Receptor (Mayo, 1992).

One potential target for genetic engineering to improve cell characteristics for protein production is hexokinase I. It now has been determined that interfering with hexokinase I function reduces the growth rate of cells. The following is a discussion of engineering of hexokinases according to the present invention.

The transcription factors NKX-2.2 and NKX-6.1 have been shown to plan an important role controlling the expression of genes involved in glucose response. Their use to engineer cells is described in U.S. Pat. No. 6,127,598 (German et al.), incorporated herein by reference. Applicants specifically contemplate combining these transcription factors and the polynucleotides encoding gene products of the present invention in multiply engineered cells.

The glucagon receptor is another molecule that may be used in combination with polynucleotides encoding gene products of the present invention to create engineered cells. U.S. Pat. Nos. 5,776,725 and 5,770,445, incorprated herein by reference, describe glucagon receptor DNAs and proteins.

Other engineering steps applicable to the present invention can be found in U.S. Pat. No. 6,110,707, incorporated herein by reference.

(v) Methods for Re-engineering Engineered Cells

In many situations, multiple rounds of iterative engineering will be undertaken in generating the final cell lines. The events that may be conducted as separate construction events include blocking expression of endogenous gene products by molecular methods (including targeting of both copies of the endogenous gene), introducing a heterologous gene, and further modification of the host cell to achieve high level expression. The particular difficulty in performing multiple steps like this is the need for distinct selectable markers. This is a limitation in that only a few selectable markers are available for use in mammalian cells and not all of these work sufficiently well for the purposes of this invention.

The present invention therefore contemplates the use of the Cre/Lox site-specific recombination system (Sauer, 1993, available through Gibco/BRL, Inc., Gaithersburg, Md.) to rescue specific genes out of a genome, most notably drug selection markers. It is claimed as a way of increasing the number of rounds of engineering. Briefly, the system involves the use of a bacterial nucleotide sequence knows as a LoxP site, which is recognized by the bacterial Cre protein. The Cre protein catalyzes a site-specific recombination event. This event is bidirectional, i.e., Cre will catalyze the insertion of sequences at a LoxP site or excise sequences that lie between two LoxP sites. Thus, if a construct containing a selectable marker also has LoxP sites flanking the selectable marker, introduction of the Cre protein, or a polynucleotide encoding the Cre protein, into the cell will catalyze the removal of the selectable marker. If successfully accomplished, this will make the selectable marker again available for use in further genetic engineering of the cell. This technology is explained in detail in U.S. Pat. No. 4,959,317, which is hereby incorporated by reference in its entirety.

It also is contemplated that a series of different markers may be employed in some situations. These markers are discussed in greater detail, below.

(vi) Other Heterologous Proteins

A variety of other different proteins can be expressed according to the present invention, in order to enhance insulin secreation, and in particular, to confer glucose sensing or response capability on host cells. These proteins can have either direct or indirect impact on these pathways, and the products that effect insulin secretion. Such proteins include leptin, insulin, glucagon-like peptide I, somatostatin, amylin, pancreastatin pancreatic peptide, leptin receptor, sulfonylurea receptor, β-cell inward rectifying channels, protein processing enzymes such as PC2 and PC3, and PAM, transcription factors such as IPF1, and metabolic enzymes such as adenosine deaminase, phenylalanine hydroxylase, glucocerebrosidase.

Engineering mutated, truncated or fusion proteins into neuroendocrine cells also is contemplated. Examples of each type of engineering resulting in secretion of a protein are given (Ferber et al., 1991; Mains et al., 1995). Reviews on the use of such proteins for studying the regulated secretion pathway are also cited (Burgess and Kelly, 1987; Chavez et al., 1994).

(vii) Genetic Constructs

Also claimed in this patent are examples of DNA expression plasmids designed to optimize production of the heterologous proteins. These include a number of enhancers/promoters from both viral and mammalian sources that drive expression of the genes of interest in neuroendocrine cells. Elements designed to optimize messenger RNA stability and translatability in neuroendocrine cells are defined. The conditions for the use of a number of dominant drug selection markers for establishing permanent, stable neuroendocrine cell clones expressing the peptide hormones are also provided, as is an element that links expression of the drug selection markers to expression of the heterologous polypeptide.

Vector Backbone. Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid encoding a particular gene is not believed to be important; so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the gene of interest. The use of other viral or mammalian cellular or bacterial phage promoters which are well-known in the art to achieve expression of a gene of interest is contemplated as well, provided that the levels of expression are sufficient for a given purpose.

By employing a promoter with well-known properties, the level and pattern of expression of the gene product following transfection can be optimized. Further, selection of a promoter that is regulated in response to specific physiologic signals can permit inducible expression of the gene product. Tables 5 and 6 list several elements/promoters which may be employed, in the context of the present invention, to regulate the expression of the gene of interest. This list is not intended to be exhaustive of all the possible elements involved in the promotion of gene expression but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Tables 5 and Table 6). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 5
ENHANCER

Immunoglobulin Heavy Chain
Immunoglobulin Light Chain
T-Cell Receptor
HLA DQ α and DQ β
β-Interferon
Interleukin-2
Interleukin-2 Receptor
Gibbon Ape Leukemia Virus
MHC Class II 5 or HLA-DRα
β-Actin
Muscle Creatine Kinase
Prealbumin (Transthyretin)
Elastase I
Metallothionein
Collagenase
Albumin Gene
α-Fetoprotein
α-Globin
β-Globin
c-fos
c-HA-ras
Insulin Neural Cell Adhesion Molecule (NCAM)
α1-Antitrypsin
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40 or CMV
Polyoma
Retroviruses
Papilloma Virus
Hepatitis B Virus
Human Immunodeficiency Virus

TABLE 6

| Element | Inducer |
|---|---|
| MT II | Phorbol Ester (TFA) Heavy metals |
| MMTV (mouse mammary tumor | Glucocorticoids |
| virus) | |
| β-Interferon | poly(rI)X |
| | poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TFA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone α | Thyroid Hormone |
| Gene | |
| Insulin E Box | Glucose |

In preferred embodiments of the invention, the expression construct comprises a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

Regulatory Elements. Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed. The inventors have employed the human Growth Hormone and SV40 polyadenylation signals in that they were convenient and known to function well in the target cells employed. Also contemplated as an element of the expression cassette is a terminator. These elements can serve to enhance message levels and to minimize read through from the cassette into other sequences.

(viii) Selectable Markers

In certain embodiments of the invention, the delivery of a nucleic acid in a cell may be identified in vitro or in vivo by including a marker in the expression construct. The marker would result in an identifiable change to the transfected cell permitting easy identification of expression. Usually the inclusion of a drug selection marker aids in cloning and in the selection of transformants, for example, neomycin, puromycin, hygromycin, DHFR, GPT, zeocin and histidinol. Alternatively, enzymes such as herpes simplex virus thymidine kinase (tk) (eukaryotic) or chloramphenicol acetyltransferase (CAT) (prokaryotic) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

(ix) Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988; Jang et al., 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

D. In Vivo Delivery and Treatment Protocols

It is proposed that engineered cells of the present invention may be introduced into animals with certain needs, such as animals with insulin-dependent diabetes. In the diabetic treatment aspects, ideally cells are engineered to achieve glucose dose responsiveness closely resembling that of islets. However, other cells will also achieve advantages in accordance with the invention. It should be pointed out that the experiments of Madsen and coworkers have shown that implantation of poorly differentiated rat insulinoma cells into animals results in a return to a more differentiated state, marked by enhanced insulin secretion in response to metabolic fuels (Madsen et al., 1988). These studies suggest that exposure of engineered cell lines to the in vivo milieu may have some effects on their response(s) to secretagogues.

The preferred methods of administration involve the encapsulation of the engineered cells in a biocompatible coating. In this approach, the cells are entrapped in a capsular coating that protects the contents from immunological responses. One preferred encapsulation technique involves encapsulation with alginate-polylysine-alginate. Capsules made employing this technique generally have a diameter of approximately 1 mm and should contain several hundred cells.

Cells may thus be implanted using the alginate-polylysine encapsulation technique of O'Shea and Sun (1986), with modifications, as later described by Fritschy et al. (1991). The engineered cells are suspended in 1.3% sodium alginate and encapsulated by extrusion of drops of the cell/alginate suspension through a syringe into CaCl₂. After several washing steps, the droplets are suspended in polylysine and rewashed. The alginate within the capsules is then reliquified by suspension in 1 mM EGTA and then rewashed with Krebs balanced salt buffer.

An alternative approach is to seed Amicon fibers with cells of the present invention. The cells become enmeshed in the fibers, which are semipermeable, and are thus protected in a manner similar to the micro encapsulates (Altman et al., 1986). After successful encapsulation or fiber seeding, the cells may be implanted intraperitoneally, usually by injection into the peritoneal cavity through a large gauge needle (23 gauge).

A variety of other encapsulation technologies have been developed that are applicable to the practice of the present invention (see, e.g., Lacy et al., 1991; Sullivan et al., 1991; WO91/10470; WO 91/10425; WO 90/15637; WO 90/02580; U.S. Pat. No. 5,011,472; U.S. Pat. No. 4,892,538; and WO 89/01967; each of the foregoing being incorporated by reference).

Lacy et. al. (1991) encapsulated rat islets in hollow acrylic fibers and immobilized these in alginate hydrogel. Following intraperitoneal transplantation of the encapsulated islets into diabetic mice, normoglycemia was reportedly restored. Similar results were also obtained using subcutaneous implants that had an appropriately constructed outer surface on the fibers. It is therefore contemplated that engineered cells of the present invention may also be straightforwardly “transplanted” into a mammal by similar subcutaneous injection.

Sullivan et. al. (1991) reported the development of a biohybrid perfused “artificial pancreas”, which encapsulates islet tissue in a selectively permeable membrane. In these studies, a tubular semi-permeable membrane was coiled inside a protective housing to provide a compartment for the islet cells. Each end of the membrane was then connected to an arterial polytetrafluoroethylene (PTFE) graft that extended beyond the housing and joined the device to the vascular system as an arteriovenous shunt. The implantation of such a device containing islet allografts into pancreatectomized dogs was reported to result in the control of fasting glucose levels in 6/10 animals. Grafts of this type encapsulating engineered cells could also be used in accordance with the present invention.

The company Cytotherapeutics has developed encapsulation technologies that are now commercially available that will likely be of use in the application of the present invention. A vascular device has also been developed by Biohybrid, of Shrewsbury, Mass., that may have application to the technology of the present invention.

Implantation employing such an encapsulation technique are preferred for a variety of reasons. For example, transplantation of islets into animal models of diabetes by this method has been shown to significantly increase the period of normal glycemic control, by prolonging xenograft survival compared to unencapsulated islets (O'Shea and Sun, 1986; Fritschy et al., 1991). Also, encapsulation will prevent uncontrolled proliferation of clonal cells. Capsules containing cells are implanted (approximately 1,000-10,000/animal) intraperitoneally and blood samples taken daily for monitoring of blood glucose and insulin.

An alternate approach to encapsulation is to simply inject glucose-sensing cells into the scapular region or peritoneal cavity of diabetic mice or rats, where these cells are reported to form tumors (Sato et al., 1962). Implantation by this approach may circumvent problems with viability or function, at least for the short term, that may be encountered with the encapsulation strategy. This approach will allow testing of the function of the cells in experimental animals but obviously is not applicable as a strategy for treating human diabetes.

Engineering of primary cells isolated from patients is also contemplated as described by Dr. Richard Mulligan and colleagues using retroviral vectors for the purposes of introducing foreign genes into bone marrow cells (see, e.g. Cone et al., 1984; Danos et al., 1988). The cells of the bone marrow are derived from a common progenitor, known as pluripotent stem cells, which give rise to a variety of blood borne cells including erythrocytes, platelets, lymphocytes, macrophages, and granulocytes. Interestingly, some of these cells, particularly the macrophages, are capable of secreting peptides such as tumor necrosis factor and interleukin 1 in response to specific stimuli. There is also evidence that these cells contain granules similar in structure to the secretory granules of β-cells, although there is no clear evidence that such granules are collected and stored inside macrophages as they are in β-cells (Stossel, 1987).

It may ultimately be possible to use the present invention in combination with that previously described by the one of the present inventors (U.S. Pat. No. 5,427,940, incorporated herein by reference) in a manner described for clonal cells to engineer primary cells that perform glucose-stimulated insulin secretion. This approach would completely circumvent the need for encapsulation of cells, since the patient's own bone marrow cells would be used for the engineering and then re-implanted. These cells would then develop into their differentiated form (i.e., the macrophage) and circulate in the blood where they would be able to sense changes in circulating glucose by secreting insulin.

Alternatively, it may be desirable to introduce genetic constructs to cells in vivo. There are a number of way in which nucleic acids may introduced into cells. Several methods are outlined below.

(i) Adenovirus

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization or adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kB of DNA. Combined with the approximately 5.5 kB of DNA that is replaceable in the E1 and E3 regions, the maximum capacity of the current adenovirus vector is under 7.5 kB, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

(ii) Retroviruses

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

(iii) Other Viral Vectors as Expression Constructs

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. recently introduced the chloramphenicol acetyltransferase (C′AT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

(iv) Non-viral Vectors

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. As described above, the preferred mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid. encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In one embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the liposome may be complexed with a hemaglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type such as lung, epithelial or tumor cells, by any number of receptor-ligand systems with or without liposomes. For example, epidermal growth factor (EGF) may be used as the receptor for mediated delivery of a nucleic acid encoding a gene in many tumor cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues. Anderson et al., U.S. Pat. No. 5,399,346, and incorporated herein in its entirety., disclose ex vivo therapeutic methods.

(v) Pharmaceutical Compositions

Where clinical applications are contemplated, it will be necessary to prepare pharmaceutical compositions—either gene delivery vectors or engineered cells—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

Solutions of the active ingredients as free base or pharmacologically acceptable salts can be prepared in water suitably mixed with surfactant, such as hydroxypropylcellulose. Dispersions also can be prepared in glycerol, liquid polyethylene glycols, mixtures thereof and in oils. Under ordinary conditions of storage and use, these preparations contain a preservative to prevent growth of microorganisms.

The expression vectors and delivery vehicles of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The vectors and cells of the present invention are advantageously administered in the form of injectable compositions either as liquid solutions or suspensions; solid forms suitable for solution in, or suspension in, liquid prior to injection also may be prepared. These preparations also may be emulsified. A typical composition for such purposes comprises a 50 mg or up to about 100 mg of human serum albumin per milliliter of phosphate-buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

E. Diagnostics

(i) Nucleic Acid Detection

In addition to their use in directing the expression of proteins, polypeptides and/or peptides, the nucleic acid sequences disclosed herein have a variety of other uses. For example, they have utility as probes or primers for embodiments involving nucleic acid hybridization in the identification of cells and organisms that overexpress, underexpress, or express variant forms of polypeptides encoded by SEQ ID NOS:1-7, 66, 67, 69, 70, 72 or 74.

Hybridization. The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

Amplification of Nucleic Acids. Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to SEQ ID NOS:1-7 are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,633,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

Detection of Nucleic Acids. Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

Other Assays. Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substititution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

Kits. All the essential materials and/or reagents required for detecting SEQ ID NOS:1-7 in a sample may be assembled together in a kit. This generally will comprise a probe or primers designed to hybridize specifically to individual nucleic acids of interest in the practice of the present invention, including SEQ ID NOS:1-7, 66, 67, 69, 70, 72 or 74. Also included may be enzymes suitable for amplifying nucleic acids, including various polymerases (reverse transcriptase, Taq, etc.), deoxynucleotides and buffers to provide the necessary reaction mixture for amplification. Such kits may also include enzymes and other reagents suitable for detection of specific nucleic acids or amplification products. Such kits generally will comprise, in suitable means, distinct containers for each individual reagent or enzyme as well as for each probe or primer pair.

(ii) Immunologic Detection

Compositions. Diagnostic procedures also may involved detection of proteins. A convenient means for selectively examining a protein encoded by SEQ ID NOS:1-7, 66, 67, 69, 70, 72 or 74 is by immonological means. For example, an antibody of sufficient selectivity, specificity or affinity may be employed as the basis for an antibody-basd detection method. Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging.”

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (HI), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens, & Haley, 1987; Atherton et al., 1985). The 2- and 8-azido nucleotides have also been used to map nucleotide binding domains of purified proteins (Khatoon et al, 1989; King et al., 1989; and Dholakia et al, 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. Nos. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

Methods. In still further embodiments, the present invention concerns immunological methods for detecting polypeptides encoded by SEQ ID NOS:1-7, 66, 67, 69, 70, 72 or 74. Antibodies to these targets, prepared in accordance with the present invention, may be employed to detect wild-type and/or mutant polypeptides, or changes in levels of expression. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H & Ben-Zeev O, 1999; Gulbis & Galand, 1993; De Jager et al., 1993; each incorporated herein by reference.

The immunobinding methods include methods for detecting and quantifying the amount of a wild-type or mutant polypeptide in a sample and the detection and quantification of any immune complexes formed during the binding process. Here, one would obtain a sample suspected of containing a wild-type or mutant polypeptide and contact the sample with an antibody that recognizes the target, and then detect and quantify the amount of immune complexes formed under the specific conditions.

In terms of antigen detection, the biological sample analyzed may be any sample that is suspected of containing a wild-type or mutant polypeptide, such as a tissue section or specimen, a homogenized tissue extract, a cell, or separated and/or purified forms of any of the above wild-type or mutant polypeptide.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes, i.e., to bind with antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. No. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

ELISAs. As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, the antibodies of the invention are immobilized onto a selected surface exhibiting protein affinity, such as a well in a polystyrene microtiter plate. Then, a test composition suspected of containing the wild-type and/or mutant polypeptide antigen is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second unlabeled antibody, followed by the addition of a third antibody that has binding affinity for the second antibody, with the third antibody being linked to a detectable label.

In another exemplary ELISA, the samples suspected of containing the wild-type and/or mutant antigen are immobilized onto the well surface and/or then contacted with the antibodies of the invention. After binding and/or washing to remove non-specifically bound immune complexes, the bound antibodies are detected. Where the initial anti-antibodies are linked to a detectable label, the immune complexes may be detected directly. Again, the immune complexes may be detected using a second antibody that has binding affinity for the first antibody, with the second antibody being linked to a detectable label.

Another ELISA in which the wild-type and/or mutant polypeptides of the invention are immobilized involves the use of antibody competition in the detection. In this ELISA, labeled antibodies against wild-type or mutant protein are added to the wells, allowed to bind, and/or detected by means of their label. The amount of protein in an unknown sample is then determined by mixing the sample with the labeled antibodies against protein before and/or during incubation with coated wells. The presence of protein in the sample acts to reduce the amount of antibody against protein available for binding to the well and thus reduces the ultimate signal. This is also appropriate for detecting antibodies against protein in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

Immunohistochemistry. The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

Immunodetection Kits. In still further embodiments, the present invention concerns immunodetection kits for use with the immunodetection methods described above. The immunodetection kits will thus comprise, in suitable container means, a first antibody that binds to a wild-type and/or mutant polypeptide.

The immunodetection reagents of the kit may take any one of a variety of forms, including those detectable labels that are associated with and/or linked to the given antibody. Detectable labels that are associated with and/or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and/or all such labels may be employed in connection with the present invention.

The container means of the kits will generally include at least one vial, test tube, flask, bottle, syringe and/or other container means, into which the antibody may be placed, and/or preferably, suitably aliquoted. The kits of the present invention will also typically include a means for containing the antibody, antigen, and/or any other reagent containers in close confinement for commercial sale. Such containers may include injection and/or blow-molded plastic containers into which the desired vials are retained.

F. Drug Screening

The present invention further comprises methods for identifying compounds that modulate the expression and/or function of polypeptides encoded by SEQ ID NOS:1-7, 66, 67, 69, 70, 72 or 74. These assays may comprise random screening of large libraries of candidate substances; alternatively, the assays may be used to focus on particular classes of compounds selected with an eye towards structural or functional attributes that are believed to make them more likely to modulate the function of the aforementioned targets.

To identify a modulator, one generally will determine the expression of target in the presence and absence of the candidate substance, a modulator defined as any substance that alters function. For example, a method generally comprises:

(a) providing a candidate modulator;

(b) admixing the candidate modulator with an isolated cell or cell population, or a suitable experimental animal;

(c) measuring expression of one or more target genes; and

(d) comparing the expression measured in step (c) with the expression of the same gene in the absence of said candidate modulator,

wherein a difference between the measured expression indicates that said candidate modulator is, indeed, a modulator of expression of the target gene or genes.

Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

1. Modulators

As used herein the term “candidate substance” refers to any molecule that may potentially inhibit or enhance expression. The candidate substance may be a protein or fragment thereof, a small molecule, or even a nucleic acid molecule. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be candidate modulators.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on the glucose sensing apparatus. Regardless of the type of inhibitor or activator identified by the present screening methods, the effect of the inhibition or activator by such a compound results in improved glucose responsiveness as compared to that observed in the absence of the added candidate substance.

2. In Cyto Assays

The present invention contemplates the screening of compounds for their ability to modulate expression in isoalted cells. Various cell lines can be utilized for such screening assays, including cells specifically engineered for this purpose.

Depending on the assay, culture may be required. The cell is examined using any of a number of different physiologic assays, especially those designed to identify whether or not the glucose sensing/responsiveness of a cell has been altered. Alternatively, molecular analysis may be performed, for example, looking at protein expression, mRNA expression (including differential display of whole cell or polyA RNA) and others.

3. In Vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

### G. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### MATERIALS AND METHODS

### Materials

All RNA was extracted using RNeasy Mini kit (QIAGEN Inc, Valencia Calif.). For cDNA: Superscript II RT kit (Life Technologies/Gibco, Inc., Rockville Md.), except as noted. All primers used were from (Life Technologies/Gibco, Inc., Rockville Md.).

For all PCRs in MPX-PCR, RDA, and RACE, the following were used: Taq DNA Polymerase+10×buffer (Promega Corp., Madison Wis.); dNTP (Life Technologies/Gibco, Inc., Rockville Md.); Mineral oil (Sigma, St. Louis Mo.); SeaKem ME agarose (FMC, Rockland Me.); PCR machine and T3 thermocycler (Biometra, Göttingen, Germany).

All DNA extraction from agarose gels used QiaQuick Gel extraction kit. PCR purification used QiaQuick PCR purification kit, both (QIAGEN Inc, Valencia Calif.).

### Cell Line Characterization

The INS-1 cell line is a rat insulin secreting cell line derived from a radiation induced rat insulinoma (Asfari et al., 1992). The cell line was genetically engineered to express the human insulin gene (Hohmeier et al., 2000; Chen et al., 2000). The clones derived from the human insulin engineering were heterogeneous with regard to magnitude of insulin secretion in response to glucose stimulation. After antibiotic selection and clonal expansion, 67% of the resultant clones were found to be poorly responsive to glucose in terms of insulin secretion (≦2-fold stimulation by 15 mM compared to 3 mM glucose), 17% of the clones were moderately responsive (2- to 5-fold stimulation), and 16% were strongly responsive (5- to 13-fold stimulation) (FIG. 1). Since the cell lines had roughly equivalent insulin content, it was hypothesized that the differences in glucose stimulated insulin secretion were due to differentially expressed genes.

### Multiplex RT-PCR

The method of Gasa et al., (2000) was followed. Briefly, cells were grown in 10 cm tissue culture dishes. RNA was extracted using the RNeasy kit (Quiagen Inc., Valencia Calif.). RNA was dissolved in dH₂O at a concentration of 0.2 μg/μl. cDNA was prepared from 1 μg of RNA using the Superscript RT kit and random hexamer primers (Life Technologies, Inc., Rockville Md.). cDNA was diluted 1:6 with dH₂O. PCR was carried out according to a protocol supplied by Promega Corporation (Promega Corp., Madison Wis.). Each reaction contained 3 μl diluted cDNA, 2.5 units Taq DNA polymerase, 40 mM each dATP, dTTP, dGTP, 20 mM dCTP (Life Technologies, Rockville, Md.), [α-³³P]dCTP (2000 Ci/mmol) (NEN Life Science Products, Boston Mass.) and primer sets (0.4 μM final concentration for each primer) in a final volume of 25 μl of 1×Taq polymerase buffer (Promega Corp., Madison, Wis.). The sequences of the oligonucleotides used as primer sets are provided in Table 7.

TABLE 7
PRIMERS USED IN RACE IN ADDITION TO THE GSP'S
5′/3′ RACE primers

| | |
|---|---|
| Oligo-dT | acgcggatccatcgatgtcgacttttttttttttttttttv |
| anchor | |
| Anchor | acgcggatccatcgatgtcgac |
| Oligo-dG | ggccacgcgtcgactagtacgggiigggiigggig |
| anchor | |
| G anchor | ggccacgcgtcgactagtac |

PRIMERS USED IN RDA

| | |
|---|---|
| R24 | agcactctccagcctctcaccgca |
| R12 | gatctgcggtga |
| N24 | aggcaactgtgctatccgagggaa |
| N12 | gatcttccctcg |
| J24 | accgacgtcgactatccatgaaca |
| J12 | gatctgttcatg |

PCR reactions were initially maintained for 2 min at 95° C., followed by 18-24 cycles of 94° C. for 45 sec to 55° C. for 45 sec to 72° C. for 45 sec. PCR reaction products were denatured with 98% formamide denaturing loading buffer, and separated on 6% (w/v) polyacrylamide gel w/7 M urea. Band image and intensity were obtained and quantified using an ImageQuant analyser (Molecular Dynamics, Sunnyvale Calif.).

### Representational Difference Analysis (RDA)

Representational difference analysis (RDA) was carried out according to the protocol of Hubank & and Schatz (1994). Total RNA was prepared from INS-1-derived cell lines grown in 10 cm dishes to 100% confluence using the RNeasy kit (QIAGEN Inc, Valencia Calif.). mRNA was prepared from 0.5-1 mg total RNA using the Oligotex kit (QIAGEN Inc, Valencia Calif.). Double stranded cDNA was prepared using oligodT hybridized to 2 μg mRNA and the Superscript Choice system for cDNA synthesis (Life Technologies Inc., Rockville, Md.). Double stranded cDNA was digested with the restriction enzyme DpnII (New England BioLabs, Beverly Mass.) by combining 1 μl DpnII (10 U/μl), 2 μg double stranded cDNA, and 1 μl 10×DpnII buffer, brought to a final volume of 10 μl with dH₂O, and incubated at 37° C. for 3 hr. The DpnII enzyme was denatured by increasing the temperature of the mixture to 65° C. for 20 min. Digested cDNA was ligated to R-12/24 linkers (Life Technologies, Rockville, Md.) by combining 5 μl of the foregoing Dpn II digested cDNA, 1 μl desalted R-24 linker (2 mg/ml), 1 μl desalted R-12 linker (1 mg/ml), 6 μl 10×ligase buffer (New England BioLabs, Beverly Mass.), and dH₂O to a final volume of 60 μl. Annealing was carried out by increasing the temperature of the mixture to 50° C., and then cooling to 10° C. over 1 hr. One μl T4 DNA ligase (400 U/μl) (New England BioLabs, Beverly Mass.) was added to the mixture, and incubated overnight at 14° C. The ligation mixture was diluted by adding 140 μl TE for a final volume of 200 μl. The representative amplicons were generated from 20 PCR reactions per cDNA sample.

For each PCR reaction, 155 μl dH₂O, 20 μl 10×PCR buffer, 16 μl nucleotide mix (4 mM each dNTP) (Life Technologies, Rockville, Md.), 2 μl R-24 primer (1 mg/ml), and 2 μl diluted ligation mixture were combined in a 0.5 ml eppendorf tube, and overlaid with mineral oil. The mixture was heated for 3 min at 72° C., 1 μl Taq DNA polymerase (5 U) was added, and incubated for an additional 5 min at 72° C. The PCR reactions were 22 cycles of 1 min at 95° C., and 3 min at 72° C. After cycling the reactions were maintained for 10 min at 72° C. and cooled to 4° C. PCR reaction products were purified using centricon-30 concentrators (Amicon Inc., Beverly Mass.) and eluted in 100 μl H₂O. The concentration of eluted samples was adjusted to 0.5 μg/μl. The DNA samples were then digested with DpnII in reactions containing 300 μl DNA solution, 70 μl DpnII buffer; 27 μl DpnII (10 U/μl), and 30 μl dH₂O, incubated at 37° C. for 5 hrs, after which the enzyme was denatured at 65° C. for 20 min. Digested products were phenol/chloroform extracted, isopropanol precipitated, and resuspended in TE at 0.2-0.3 μg DNA/μl. This sample was used as the “driver.” The “tester” was prepared by running 40 μl digested DNA, 30 μl TE, and 14 μl 6×loading buffer over a 1.2% agarose/TBE prep gel. Tester DNA separated from the linkers was extracted from the gel slice using QIAquick Gel Extraction Kit (QIAGEN Inc, Valencia Calif.) and eluted in 120 μl TE. The tester was ligated to J-oligos in a reaction mixture that contained 2 μg gel purified tester; 6 μl 10×ligase buffer, 4 μl desalted J-24 (2 mg/ml), and 4 μl desalted J-12 (1 mg/ml) brought to a final volume of 57 μl with dH₂O. Annealing was carried out by heating the DNA to 50° C. for 1 min and cooling to 10° C. at 1° C. per min. Three μl T4 DNA ligase (400 U/ml) was added, and incubated at 12-14 ° C. overnight. For subtractive hybridization, 25 μl driver DNA at a concentration of 200-300 ng/μl was mixed with 10 μl J-ligated tester (0.25-0.3 μg) and 75 μl TE. The driver/tester DNA mixture was phenol/CHCl₃ extracted, ethanol precipitated, and resuspended in 4 μl EE×3 buffer. The driver/tester DNA mixture was overlaid with mineral oil and denatured for 5 min at 98° C. The mixture was rapidly cooled to 67° C., and 1 μl 5 M NaCl was added immediately. The mixture was hybridized at 67° C. for 20 hrs, after which the surface mineral oil was removed and 400 μl TE was added to dilute the hybridization mix.

To generate the first difference products (DP1), five PCR reactions for each hybridization were set up. For each PCR reaction, 140 μl dH₂O, 20 μl 10×PCR buffer, 16 μl dNTPs (4 mM each), 4 μl MgCl₂ (25 mM), and 20 μl diluted hybridization mix were combined in a 0.5 ml eppendorf tube. The PCR mixture was denatured for 3 min at 72° C. One μl (5 U) Taq DNA polymerase was added and the mixtures were further incubated at 72° C. for 5 min. 2.5 μl J-24 primer (100 μM) was added, and the PCR reactions were run for 11 cycles of 1 min at 95° C. to 3 min at 70° C. Final extension was carried out for 10 min at 72° C., followed by cooling to room temperature. The five PCR reactions were combined, phenol/CHCl₃ extracted, CHCl₃ extracted, isopropanol precipitated, and resuspended in 40 μl 0.2×TE. The PCR products were then digested by mixing 20 μl DNA, 4 μl 10×Mung Bean Nuclease buffer, 14 μl dH₂O, and 2 μl Mung Bean Nuclease (10 U/μl; NEB) at 30° C. for 35 min. The enzyme was inactivated using 160 μl 50 mM Tris-HCl (pH 8.9) heated to 98° C. for 5 min, followed by chilling on ice. For each subtraction, another 4 PCR reactions were set up. Each PCR reaction contained a mixture of 137 μl dH₂O; 20 μl 10×PCR buffer; 16 μl dNTPs (4 mM each); 2.5 μl J-24 (100 μM); 20 μl MBN treated DNA. The PCR mixture was heated for 1 min to 95° C., and cooled to 80° C. before 1 μl Taq DNA polymerase (5 U) was added. PCR was performed for 18 cycles of 1 min at 95° C., 3 min at 70° C., followed by 10 min of extension at 72° C. and cooling to 4° C. PCR DNA products were combined, phenol/CHCl₃ extracted, CHCl₃ extracted, isopropanol precipitated, and resuspended in 100 μl 0.2×TE. The J-adaptors of the first difference product were changed to N-adaptors. To remove the J-adaptors, 20 μl (10 μg) difference product 1 (DP1) DNA, 30 μl 10×DpnII buffer, 7.5 μl Dpn II (75 U), and 242.5 μl dH₂O were combined. The digestion reaction was carried out for 3h at 37° C.

DNA digestion products were phenol/CHCl₃ extracted, CHCl₃ extracted, ethanol precipitated, and resuspended in 20 μl TE (0.2-0.3 mg/ml). One μl DP1 (200-300 ng), 6 μl 10×ligase buffer; 10 μl desalted N-24 (100 mM), 10 μl desalted N-12 (100 mM), and 30 μl dH₂O were combined. The mixture was annealed by incubating at 50° C. for 1 min, then cooled to 10° C. at 1° C. per min. Three μl T4 DNA ligase (400 U/μl) was added to the mixture, and the ligation reaction was carried out for 18 hrs at 12-14° C., after which the DP1/N-adapter ligation mixture was diluted with TE to a final concentration of 1.25 ng DNA/μl.

To generate the second difference product (DP2), 12 μl DP1/N-adapter ligation (15 ng) and 40 μl driver DNA (12 μg) were combined. The remainder of the subtractive hybridization and PCR amplification was the same as for the first difference product except that the annealing temperature in the PCR was 72° C. To generate the third difference product (DP3) the linker on DP2 was changed from N to J as before. Then 1.5 μl DP2/J-adaptor ligation (0.3 ng) was mixed with 40 μl driver (12 μg) (1:40.000), and put through the same subtractive hybridization and PCR amplification reactions described for DP1, except that the last PCR was performed for 23 cycles at 95° C. 1 min, 70° C. 3 min, with a final extension at 72° C. for 10 min.

### Cloning and Sequencing of DP2 and DP3

P2 and DP3 were digested with Dpn II as above, and separated on a 1.2 % agarose gel. Individual bands were cut out, purified using Qiaquick gel extraction kit, and ligated to Bam HI digested and phosphatase treated pBluescript II in an overnight ligation using T4 DNA ligase. Competent DHα5 cells (Invitrogen, Carlsbad Calif.) were transformed with the ligated vectors and plated on LB/ampicilin plates prepared for blue/white selection using IPTG and x-gal. White colonies were picked and screened using PCR (primers T3 and T7) for inserts in the vector. Positive colonies were amplified and minipreps prepared. The resulting plasmids were sequenced using T7 and T3 primers on an automated ABI Prism 377 DNA Sequencer at the sequencing facility, Dept. of Biochemistry, University of Texas Southwestern Medical Center at Dallas.

### Cloning Unknown Genes and ESTs using RACE PCR

Unknown genes and ESTs were cloned using the 3′ and 5′ RACE System for Rapid Amplification of cDNA Ends kits, Version 2.0, (Life Technologies, Inc., Rockville Md.), with minor modifications as noted, and the First Choice RLM-Race kit (Ambion Inc., Austin Tex.). RNA for cDNA synthesis was extracted from cells grown to near confluency in 10 cm tissue culture dishes using the RNeasy kit (Quiagen Inc., Valencia Calif.).

First strand cDNA for 3′ RACE was synthesized using 1 μg total RNA mixed with 2 μl Oligo-dT anchor primer (20 μM) and H₂O to a final volume of 10 μl, heated to 70° C. for 10 min and then chilled on ice. To this was added a 10 μl mix consisting of 2 μl 5×1st strand buffer, 1 μl 0.1 M DTT, 1 μl RNAse-out, 2 μl dNTP mix (10 mM each) and 1 μl Superscript II reverse transcriptase (all Life Technologies Inc., Rockville Md.). Samples were incubated for 15 min at 37° C., 45 min at 45° C., and 10 min at 50° C., and reactions terminated by heating to 95° C. for 3 min. One μl of RNase H was added and the mixture was incubated for 20 min at 37° C., followed by termination of the reaction by heating for 10 min at 70° C. The following PCR was carried out using the anchor primer specific for the 3′-end of the oligo-dT anchor primer and a gene specific primer (GSP.s) pointing in one direction arbitrarily called the sense primer or an “antisense” GSP (GSP.a) pointing in the opposite direction. Using two primers pointing in either direction in two PCR reactions allows for the determination of the sense and anti sense orientation of the gene. The PCR mixture consisted of 5 μl 10×PCR buffer; 1 μl 10 mM dNTP mix; 1 μl 20 μM GSP.s or GSP.a, 1 μl 20 μM anchor primer, 0.75 μl of 5U/μl Taq DNA polymerase, and ddH₂O to a final volume of 49 μl. The mixture was overlaid with mineral oil and then incubated at 95° C. One μl first strand DNA was added (hotstart) and denatured for 2 min at 95° C. PCR amplification was conducted for 10 cycles of 94° C. for 1 min, 55° C. for 1 min, 72° C. for 2 min, followed by 25 cycles in which the elongation step was increased by 5 sec for each additional round. A final extension of 72° C. for 7 min was applied, and samples were stored at 4° C. PCR products were separated on a 1% agarose gel. PCR fragments of interest were cut out and purified using QUAquick Gel Extraction kit (Quiagen Inc., Valencia Calif.).

The fragment was either sequenced directly using the GSP or cloned using the TA cloning kit (Invitrogen, Carlsbad Calif.). DNA sequencing was performed at the Department of Biochemistry DNA Sequencing Laboratory at the University of Texas Southwestern Medical Center at Dallas by mixing 100 ng PCR DNA or 1 μg of plasmid and 16 p moles of oligonucleotide for sequencing on an ABI Prism 377 DNA Sequencer. For all genes a nested PCR was performed on either total PCR products (purified using the Qiaquick PCR purification kit (Qiagen)) or purified PCR fragments using a second GSP positioned 3′ to the first primer called GSP.s2 or GSP.a2, and the anchor primer. PCR scheme; add DNA at 95° C. to denature for 2 min, then 25 cycles of 94° C. for 45 sec, 55 or 58° C. for 45 sec, 72° C. for 2 min. Final extension was made at 72° C. for 7 min 2° C. PCR treated as for the first PCR.

For 5′ RACE, the 5′ RACE kit instructions (Life Technologies, Inc., Rockville Md.) were followed with minor deviations. First strand cDNA synthesis was carried out as for the 3′ RACE except for the use of a GSP for the initiation of 1 st.synthesis. Instead of Superscript II, Thermoscript (Life Technologies, Inc., Rockville Md.) was used. Thermoscript RT works at temperatures of up to 60° C. and allows for a more specific annealing of the GSP. Five μg of RNA was used with 0.5 μl GSP (20 μM), and after denaturation, the RNA was kept at 55° C. and the reaction mix was heated to 55° C. before being added to the RNA. The reaction was carried out at 55° C. for 60 min, then 60° C. for 10 min. This protocol will also minimize secondary structures. To terminate the reaction, samples were incubated at 85° C. for 10 min. One μl RNase mix was then added and samples were incubated at 37° C. for 30 min, followed by termination at 70° C. for 10 min. The cDNA was purified using QIAquick PCR purification kit and eluted in 30 μl elution buffer. After 1st strand cDNA synthesis a poly-C tail was added using the TdT kit (NEB, Beverly Mass.). The reaction mixture contained 2.5 μl 10×buffer; 2.5 μl CoCl₂ (10×), 1 μl 5 mM dCTP, 10 μl purified cDNA, and dH₂O to 25 μl. The mixture was incubated at 85° C. for 3 min, cooled to 37° C., and 1 μl TdT was added, and the reaction was carried out at 37° C. for 20 min. The TdT was heat inactivated at 70° C. for 10 min, and placed on ice. The 5′ RACE-PCR reaction was carried out as for the 3′ RACE-PCR except for the use of a oligo-dG anchor primer and a GSP primer 3′ to the primer used for 1^{st} strand cDNA synthesis. PCR fragments of interest was treated as for 3′ RACE. Nested PCR was carried out using a third GSP (GSP.s3 or GSP.a3) and a “G-anchor” primer, and amplified fragments were subjected to DNA sequencing as above. If the 5′ end of the gene was not reached, a new gene specific primer nested in the region of newly sequenced DNA was synthesized and the whole process repeated.

Unknown genes and ESTs were also cloned using the 5′ and 3′ RLM-RACE Protocol kit (Ambion Inc., Austin Tex.). For the 5′ RLM-RACE, 10 μg total cell RNA; 2 μl 10×calf intestinal phosphatase (CIP) buffer; 2 μl CIP; and dH₂O were combined in a final volume 20 μl and reacted at 37° C. for 1 hr. CIP'd RNA was phenol extracted, ethanol precipitated, resuspended in 11 μl TE (1.1 μg/ml). Five μl CIP'd RNA; 1 μl 10×tobacco acid pyrophosphatase (TAP) buffer; 2 μl TAP; and 2 μl dH₂O were mixed, and reacted at 37° C. for 1 hr. To ligate the 5′ RACE adapter, 2 μl CIP/TAP-treated RNA; 1 μl 5′ RACE adapter; 1 μl 10×RNA ligase buffer; 2 μl (5 U) T4 RNA ligase, and 4 μl dH₂O were mixed, and reacted at 37° C. for 1 hr. For reverse transcription, 2 μl ligated RNA; 4 μl dNTP mix; 2 μl random decamers; 2 μl 10×RT buffer; 1 μl Rnase inhibitor; 1 μl MMLV reverse transcriptase, and dH₂O were combined to a final volume 20 μl, and reacted at 42° C. for 1 hr.

To amplify a gene-specific 5′ fragment, a nested PCR protocol, which utilizes 2 nested primers corresponding to the 5′ RACE Adapter sequence and 2 nested gene-specific primers (GSP), was employed. For the initial amplification, 1 μl RT reaction, 2 μl 10×PCR buffer, 2 μl 8 mM (2 mM each) dNTP mix, 0.6 μl 50 mM MgCl₂, 0.8 μl 10 μM 5′ RACE outer primer, 2 μl 4 μl 5′ outer GSP, 0.3 μl (1.5 U) Taq Polymerase (Life Technologies, Inc.) and 11.3 μl dH₂O were mixed. To amplify, the mixture was subjected to 35 cycles having the following parameters: denature, 30 seconds at 95° C., anneal, 30 seconds at 55-60° C., and elongate for 45 seconds to 1.5 minutes at 72 ° C. The reactions was completed by a 7 minute incubation at 72° C.

For the second (i.e., “nested”) amplification, 1 μl “outer” 5′ RACE PCR reaction, 2 μl 10×PCR buffer, 2 μl 8 mM (2 mM each) dNTP mix, 0.6 μl 50 mM MgCl₂, 0.8 μl 10 μM 5′ RACE inner primer, 2 μl 4 μM 5′ inner GSP, 0.3 μl (1.5 U) Taq Polymerase (Life Technologies, Inc.) and 11.3 μl dH₂O were mixed. The PCR denaturation, cycling, and final extension protocol was the same as for the 5′ outer primer PCR previously described. Gel separation, analysis, and sequencing of PCR product were performed as described for the Life Technologies kit.

For the 3′ RLM-RACE Protocol (Ambion Inc., Austin Tex.), 2 μl total cellular RNA (1 μg); 4 μl dNTP mix; 2 μl 3′ RACE adapter; 2 μl 10×reverse transcriptase (RT) buffer; 1 μl RNase inhibitor; 1 μl MMLV RT; and 8 μl dH₂O were mixed. The mixture was reacted for 1 hr at 42° C.

To amplify a gene-specific 3′ fragment, 1 μl RT reaction, 2 μl 10×PCR buffer, 2 μl 8 mM (2 mM each) dNTP mix, 0.6 μl 50 mM MgCl₂, 0.8 μl 10 μM 3′ RACE outer primer, 2 μl 4 μM 3′ outer GSP, 0.3 μl (1.5 U) Taq Polymerase (Life Technologies, Inc.) and 11.3 μl dH₂O were mixed. The PCR denaturation, cycling, and final extension protocol was the same as for the 5′ outer primer PCR previously described. Gel separation, analysis, and sequencing of PCR product were performed as described for the Life Technologies kit.

If necessary an inner (i.e., “nested”) amplification was performed. In this case, 1 μl “outer” 3′ RACE PCR reaction, 2 μl 10×PCR buffer, 2 μl 8 mM (2 mM each) dNTP mix, 0.6 μl 50 mM MgC₂, 0.8 μl 10 μM 3′ RACE inner primer, 2 μl 4 μM 3′ inner GSP, 0.3 μl (1.5 U) Taq Polymerase (Life Technologies, Inc.) and 11.3 μl dH₂O were mixed. The PCR denaturation, cycling, and Final extension protocol was the same as for the 5′ outer primer PCR previously described. Gel separation, analysis, and sequencing of PCR product were performed as described for the Life Technologies kit.

All of the COMPOSITIONS and METHODS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS and METHODS and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defmed by the appended claims.

KEY TO SEQUENCE LISTING

| SEQ | | |
|---|---|---|
| ID | | |
| NO | IDENTIFICATION | OTHER* |
| 1 | Clone 8 | starts: 296-298; 706-708; 866- |
| | | 868 |
| | | stops: 26-28; 33-35; 61-63; |
| | | 740-742; 880-882; 1001-1003 |
| 2 | Clone 12a | starts: 306-308 |
| | | stops: 67-69; 369-371 |
| 3 | Clone 17 | starts: 187-189; 365-367; 389- |
| | | 391; 482-484; 682-684 |
| | | stops: 3-5; 44-46; 109-111; |
| | | 346-348; 455-457; 573-575; |
| | | 790-792; 926-928 |
| 4 | Clone 27 | starts: 77-79; 209-211; 405- |
| | | 407; 588-590; 715-717; 999- |
| | | 1001; 1137-1139 |
| | | stops: 188-190; 320-322; 373- |
| | | 375; 585-587; 738-740; 805- |
| | | 807 |
| 5 | Clone 28 | starts: 130-132; 137-139; 192- |
| | | 194; 820-822 |
| | | stops: 5-7; 49-51; 120-122; |
| | | 205-207; 288-290; 461-463; |
| | | 949-951 |
| 6 | Clone 58 | starts: 166-168 |
| | | stops: 18-20; 71-73; 87-89; |
| | | 227-279 |
| 7 | Clone 60 | starts: 221-223 |
| | | stops: 77-79 |
| 8 | oligo 8.s1 | |
| 9 | oligo 8.s2 | |
| 10 | oligo 8.s3 | |
| 11 | oligo 8.s4 | |
| 12 | oligo 8.s5 | |
| 13 | oligo 8.a1 | |
| 14 | oligo 8.a2 | |
| 15 | oligo 8.a3 | |
| 16 | oligo 8.a4 | |
| 17 | oligo 8.a5 | |
| 18 | oligo 12.s1 | |
| 19 | oligo 12.s2 | |
| 20 | oligo 12.s3 | |
| 21 | oligo 12.s4 | |
| 22 | oligo 12.a1 | |
| 23 | oligo 12.a2 | |
| 24 | oligo 12.a3 | |
| 25 | oligo 12.a41 | |
| 26 | oligo 17.sense | |
| 27 | oligo 17.newsense | |
| 28 | oligo 17.5′97-.s | |
| 29 | oligo 17.3′567-.s | |
| 30 | oligo 17.3′1034-.s | |
| 31 | oligo 17.3′1313-.s | |
| 32 | oligo 17.a-sense | |
| 33 | oligo 17.a2 | |
| 34 | oligo 17.a3(EcoRI) | |
| 35 | oligo 17.3′-26.a | |
| 36 | oligo 17.5′-78.a | |
| 37 | oligo 17.3′-588.a | |
| 38 | oligo 17.3′-687.a | |
| 39 | oligo 27.newsense | |
| 40 | oligo 27.s3 | |
| 41 | oligo 27.s4 | |
| 42 | oligo 27.s5 | |
| 43 | oligo 27.a1 | |
| 44 | oligo 27.a2 | |
| 45 | oligo 27.a3 | |
| 46 | oligo 27.a4 | |
| 47 | oligo 27.a5 | |
| 48 | oligo 28.s1 | |
| 49 | oligo 28.s2 | |
| 50 | oligo 28.s3 | |
| 51 | oligo 28.s4 | |
| 52 | oligo 28.s5 | |
| 53 | oligo 28.a1 | |
| 54 | oligo 28.a2 | |
| 55 | oligo 28.a3 | |
| 56 | oligo 28.a4 | |
| 57 | oligo 28.a5 | |
| 58 | oligo 58.s1 | |
| 59 | oligo 58.s2 | |
| 60 | oligo 58.s3 | |
| 61 | oligo 58.a1 | |
| 62 | oligo 58.a2 | |
| 63 | oligo 58.a3 | |
| 64 | oligo 60.s1 | |
| 65 | oligo 60.a1 | |
| 66 | Clone 8 5′ portion | |
| 67 | Clone 8 3′ portion | |
| 68 | Murine Ocat cDNA | |
| 69 | Clone 12a 5′ portion | |
| 70 | Clone 12a 3′ portion | |
| 71 | Human clone 12a | |
| 72 | Human clone 12a cDNA | |
| 73 | Human clone 12a protein | |
| 74 | Rat/Human 12a chimeric cDNA | |
| 75 | Rat/Human 12a chimeric protein | |

*start and stop sites based on minimum 25 residue open reading frames (20 residues for clone 12a) with largest ORF indicated by underlined start/stop pair

### REFERENCES

The following references, to the extent that they provide exemplary procedural or other details supplementary to those set forth herein, are specifically incorporated herein by reference.

U.S. Pat. No. 3,817,837

U.S. Pat. No. 3,817,837

U.S. Pat. No. 3,850,752

U.S. Pat. No. 3,850,752

U.S. Pat. No. 3,939,350

U.S. Pat. No. 3,939,350

U.S. Pat. No. 3,996,345

U.S. Pat. No. 3,996,345

U.S. Pat. No. 4,275,149

U.S. Pat. No. 4,275,149

U.S. Pat. No. 4,277,437

U.S. Pat. No. 4,277,437

U.S. Pat. No. 4,352,883

U.S. Pat. No. 4,366,241

U.S. Pat. No. 4,366,241

U.S. Pat. No. 4,472,509

U.S. Pat. No. 4,472,509

U.S. Pat. No. 4,683,195

U.S. Pat. No. 4,683,202

U.S. Pat. No. 4,800,159

U.S. Pat. No. 4,883,750

U.S. Pat. No. 4,892,538

U.S. Pat. No. 4,938,948

U.S. Pat. No. 4,938,948

U.S. Pat. No. 4,938,948

U.S. Pat. No. 4,946,773

U.S. Pat. No. 5,021,236

U.S. Pat. No. 5,196,066

U.S. Pat. No. 5,279,721

U.S. Pat. No. 5,399,346

U.S. Pat. No. 5,770,445

U.S. Pat. No. 5,776,725

U.S. Pat. No. 5,840,873

U.S. Pat. No. 5,843,640

U.S. Pat. No. 5,843,650

U.S. Pat. No. 5,843,651

U.S. Pat. No. 5,843,663

U.S. Pat. No. 5,846,708

U.S. Pat. No. 5,846,709

U.S. Pat. No. 5,846,717

U.S. Pat. No. 5,846,726

U.S. Pat. No. 5,846,729

U.S. Pat. No. 5,846,783

U.S. Pat. No. 5,849,481

U.S. Pat. No. 5,849,483

U.S. Pat. No. 5,849,486

U.S. Pat. No. 5,849,487

U.S. Pat. No. 5,849,497

U.S. Pat. No. 5,849,546

U.S. Pat. No. 5,849,547

U.S. Pat. No. 5,851,770

U.S. Pat. No. 5,851,772

U.S. Pat. No. 5,853,990

U.S. Pat. No. 5,853,992

U.S. Pat. No. 5,853,993

U.S. Pat. No. 5,856,092

U.S. Pat. No. 5,858,652

U.S. Pat. No. 5,861,244

U.S. Pat. No. 5,863,732

U.S. Pat. No. 5,863,753

U.S. Pat. No. 5,866,331

U.S. Pat. No. 5,866,337

U.S. Pat. No. 5,866,366

U.S. Pat. No. 5,882,864

U.S. Pat. No. 5,900,481

U.S. Pat. No. 5,905,024

U.S. Pat. No. 5,910,407

U.S. Pat. No. 5,912,124

U.S. Pat. No. 5,912,145

U.S. Pat. No. 5,912,148

U.S. Pat. No. 5,916,776

U.S. Pat. No. 5,916,779

U.S. Pat. No. 5,919,626

U.S. Pat. No. 5,919,630

U.S. Pat. No. 5,922,574

U.S. Pat. No. 5,925,517

U.S. Pat. No. 5,925,525

U.S. Pat. No. 5,928,862

U.S. Pat. No. 5,928,869

U.S. Pat. No. 5,928,870

U.S. Pat. No. 5,928,905

U.S. Pat. No. 5,928,906

U.S. Pat. No. 5,929,227

U.S. Pat. No. 5,932,413

U.S. Pat. No. 5,932,451

U.S. Pat. No. 5,935,791

U.S. Pat. No. 5,935,825

U.S. Pat. No. 5,939,291

U.S. Pat. No. 5,942,391

U.S. Pat. No. 5,011,472

Abbondanzo et al., Am. J Clin. Pathol., (5):698-702, 1990.

Adams et al., “Porin interaction with hexokinase and glycerol kinase: Metabolic microcompartmentation at the outer mitochondrial membrane,” Bioch. Med. Met. Biol., 45:271-291, 1991.

Aguilar-Bryan et al., “Cloning of the Beta cell high-affinity sulfonylurea receptor: A regulator of insulin secretion,” Science, 268:423-426, 1995.

Ahlgren, Pfaff, Jessell, Edlund, Edlund, Nature, 385:257-260, 1997.

Allred et al., Arch Surg. 125(1):107-13, 1990.

Altman et al, Diabetes 35:625-633, 1986.

Anderson et al., “Cloning, structure, and expression of the mitochondrial cytochrome P-450 sterol 26-hydroxylase, a bile acid biosynthetic enzyme,” J.B.C., 264:8222-8229, 1989.

Antinozzi, Segall, Prentki, McGarry, Newgard, “Molecular or pharmacologic perturbation of the link between glucose and lipid metabolism is without effect on glucose-stimulated insulin secretion. A re-evaluation of the long-chain acyl-CoA hypothesis,” J Biol Chem, 273:16146-16154, 1998.

Arimura et al., “Regulation of growth hormone secretion,” The Pituitary Gland, 2nd Edition, Chap. 9:217-259. Imura (Ed.), Raven Press, N.Y., 1994.

Arora et al. J. Biol. Chem., 268:18259-18266, 1993.

Asfari et al., “Establishment of 2--mercaptoethanol-dependent differentiated insulin-secreting cell lines.” Endocrinology, 130:167-178, 1992.

Atherton et al., Biol. of Reproduction, 32:155-171, 1985.

Bahnemann et al. “Animal cell, viral antigens and vaccines,” Abs. Pap. ACS, 180:5. 1980.

Baichwal et al., “Vectors for gene transfer derived from animal DNA viruses: Transient and stable expression of transferred genes,” In: Kucherlapati (Ed.), Gene transfer, New York: Plenum Press, pp. 117-148, 1986.

Baijal, et al., J.E. Arch. Bioch. Biophys. 298:271-278, 1992.

Bailey et al., “Methylmercury as a reversible denaturing agent for agarose gel electrophoresis,” Anal. Biochem., 70:75-85, 1976.

Barr et al., “Systemic delivery of recombinant proteins by genetically modified myoblasts,” Science, 254:1507-1509, 1991.

Bassel-Duby, Hernandez, Gonzalez, Krueger, Williams, “A 40 kilodalton protein binds specifically to an upstream sequence element essential for muscle-specific transcription of the human myglobin promoter,” Mol. Cell. Biol., 12: 5024-5033, 1992.

Bauchwitz and Holloman, “Isolation of the REC2 gene controlling recombination in Ustilago maydis,” Gene, 96:285-288, 1990.

Becker et al., “Differential effects of overexpressed glucokinase and hexokinase I in isolated islets: Evidence for functional segregation of the high and low Kₘ enzymes,” J. Biol. Chem., 271:390-394, 1996.

Becker et al, “Overexpression of hexokinase 1 in isolated islets of langerhans via recombinant adenovirus,” J.B.C., 269:21234-21238, 1994.

Becker et al., “Use of recombinant adenovirus for metabolic engineering of mammalian cells,” Methods in Cell Biology, 43:161-189, Roth, M., Ed. New York, Academic Press, 1994.

Bell et al., “Characterization of the 56-kDa subunit of yeast trehalose-6-phosphate synthase and cloning of its gene reveal its identity with the product of CIF1, a regulator of carbon catabolite inactivation,” Eur. J. Biochem., 209:951-959, 1992.

Bell et al., “Nucleotide sequence of a cDNA clone encoding human preproinsulin.” Nature 282:525-527, 1979.

Benjannet et al., “Comparative biosynthesis, covalent post-translational modifications and efficiency of prosegmant cleavage of the prohormone convertases PC1 and PC2: Glycosylation, sulphation and identification of the intracellular site of prosegment cleavage of PC1 and PC2,” Biochem. J., 294:735-743, 1993.

Benvenisty and Neshif, “Direction introduction of genes into rats and expression of the genes,” Proc. Nat'l Acad. Sci. USA, 83:9551-9555, 1986.

Berberian et al., Science 261:1588-1591, 1993.

Berzal-Herranz et al., Genes and Devel., 6:129-134, 1992.

Blasquez et al., “Trehalose-6-phosphate, a new regulator of yeast glycolysis that inhibits hexokinases,” FEBS Lett., 329:51-54, 1993.

Bradford, “A rapid and sensitive method for the quantitation of microgram quantities of protein utilizing the principle of protein-dye binding,” Anal. Biochem., 72:248-254, 1976

Brewer, “Cytomegalovirus plasmid vectors for permanent lines of polarized epithelial cells,” In: Methods in Cell Biology, 43:233-245, Roth, M., Ed. New York, Academic Press, 1992.

Brunstedt et al., “Direct effect of glucose on the preproinsulin mRNA level in isolated pancreatic islets,” B.B.R.C., 106:1383-1389, 1982.

Burch et al., “Adaptation of glycolytic enzymes. Glucose use and insulin release during fasting and refeeding.” Diabetes 30:923-928.

Burgess et al., “Constitutive and regulated secretion of proteins,” Ann. Rev. Cell Biology, 3:243-293, 1987.

Cech et al., “In vitro splicing of the ribosomal RNA precursor of Tetrahymena: involvement of a guanosine nucleotide in the excision of the intervening sequence,” Cell, 27:487-496, 1981.

Challita et al., “Lack of expression from a retroviral vector after transduction of murine hematopoietic stem cells is associated with methylation in vivo,” Proc. Nat'l Acad. Sci. USA, 91:2567-2571, 1994.

Chang et al., “Foreign gene delivery and expression in hepatocytes using a hepatitis B virus vector,” Hepatology, 14:134A, 1991.

Chavez et al., “Expression of exogenous proteins in cells with regulated secretory pathways,” In: Methods in Cell Biology, 43:263-288, Roth, M., Ed. New York, Academic Press, 1994.

Chen and Okayama, “High-efficiency transfection of mammalian cells by plasmid DNA,” Mol. Cell Biol., 7:2745-2752, 1987.

Chen et al., “Regulated Secretion of Prolactin by the mouse cell line Beta TC-3,” Biotechnology, 13:1191-1197, 1995.

Chen et al., Diabetes 49:562-570, 2000.

Chowrira et al., “In vitro and in vivo comparison of hammerhead, hairpin, and hepatitis delta virus self-processing ribozyme cassetyes,” J. Biol. Chem., 269:25856-25864, 1994.

Chowrira et al., Biochemistry, 32:1088-1095.

Clark et al., “Islet cell culture in defined serum-free medium,” Endocrinology, 126:1895-1903, 1990.

Clark et al., “Modulation of glucose-induced insulin secretion from a rat clonal beta-cell line,” Endocrinology, 127:2779-2788, 1990.

Clark et al., “Novel insulinoma cell lines produced by iterative engineering of GLUT-2, glucokinase, and human insulin expression,” Diabetes, 1997.

Clark et al., “Novel insulinoma cell lines produced by iterative engineering of GLUT2, glucokinase, and human insulin expression,” Diabetes, 46:958-967, 1997.

Cleary et al., Trends Microbiol.., 4:131-136, 1994.

Coffin, “Retroviridae and their replication,” In: Virology, Fields et al. (eds.), New York: Raven Press, pp. 1437-1500, 1990.

Cole-Strausset al., “Correction of the mutation responsible for sickle cell anemia by and RNA-DNA oligonucleotide,” Science, 273:1386-1389, 1996.

Cone et al., Proc. Nat'l Acad. Sci. U.S.A. 81:6349-6353, 1984

Cordell et al., “Isolation and characterization of a cloned rat insulin gene.” Cell, 18:533-543, 1979.

Couch et al., “Immunization with types 4 and 7 adenovirus by selective infection of the intestinal tract,” Am. Rev. Resp. Dis., 88:394-403, 1963.

Coupar et al., “A general method for the construction of recombinant vaccinia virus expressing multiple foreign genes,” Gene, 68:1-10, 1988.

Curry, “Insulin content and insulinogenesis by the perfused rat pancreas: Effects of long term glucose stimulation,” Endocrinology, 118;170-175, 1986.

Cuttitta, “Peptide amidation: Signature of bioactivity,” The Anatomical Record, 236:87-93, 1993.

Dai et al., “Cellular and humoral immune responses to adenoviral vectors containing factor IX gene: Tolerization of factor IX and vector antigens allows for long-term expression,” Proc. Nat'l Acad. Sci. USA, 92:1401-1405, 1995.

Danos et al., Proc. Nat'l Acad. Sci. U.S.A. 85:6460-6464, 1988.

Day et al., “Distribution and regulation of the prohormone convertases PC1 and PC2 in the rat pituitary” Molecular Endocrinology, 6:485-497, 1992.

De Jager et al.,_“Current status of cancer immunodetection with radiolabeled human monoclonal antibodies,” Semin. Nucl. Med., 23(2):165-179, 1993.

Dhawan et al., “Systemic delivery of human growth hormone by injection of genetically engineered myoblasts,” Science, 254:1509-1512, 1991.

Dholakia et al., J. Biol. Chem., 264, 20638-20642, 1989.

Dickerson et al., “Transfected human Neuropeptide Y cDNA expression in mouse pituitary cells,” J.B.C., 262:13646-13653, 1987.

Doolittle M H and Ben-Zeev O,_“Immunodetection of lipoprotein lipase: antibody production, immunoprecipitation, and western blotting techniques” Methods Mol Biol., 109:215-237, 1999.

Drucker et al., “Cell-specific post-translational processing of preproglucagon expressed from a metallothionein-glucagon fusion gene,” J.B.C., 261:9637-9643, 1986.

Dubensky et al., “Direct transfection of viral and plasmid DNA into the liver or spleen of mice,” Proc. Nat'l Acad. Sci. USA, 81:7529-7533, 1984.

Ebbinghaus, S. W., Vigneswaran, N., Miller, C. R., Chee-Awai, R. A., Mayfield, C. A., Curiel, D. T., and Miller, D. M. 1996. Efficient delivery of triplex forming oligonucleotides to tumor cells by adenovirus-polylysine complexes. Gene Therapy 3: 287-297.

Edlund, Diabetes, 47:1817-1823, 1998.

Efrat and Fleischer, “Engineering the Pancreatic β-cell, in Diabetes mellitus: a fundamental and clincial text, LeRoith, Taylor, Olefsky, (Eds), Lippincott-Raven, N.Y., pp. 438-442, 1996.

Efrat et al., “Beta-cell lines derived from transgenic mice expressing a hybrid insulin gene- oncogene,” P.N.A.S., 85:9037-9041, 1988.

Efrat et al., “Conditional transformation of a pancreatic beta cell line derived from transgenic mice expressing a tetracycline-regulated oncogene,” Proc. Nat'l Acad. Sci., USA, 92: 3576-3580, 1995.

Efrat et al., “Murine insulinoma cell line with normal glucose-regulated insulin secretion,” Diabetes, 42:901-907, 1993.

Efrat et al., “Ribozyme-mediated attenuation of pancreatic β-cell glucokinase expression in transgenic mice results in impaired glucose-induced insulin secretion,” Proc. Nat'l Acad. Sci. USA, 91:2051-2055, 1994.

Eipper et al., “Alternative splicing and endoproteolytic processing generate tissue-specific forms of pituitary peptidylglycine alpha-amidating monooxygenase (PAM),” J.B.C., 267:4008-4015, 1992b.

Eipper et al., “The biosynthesis of neuropeptides: Peptide alpha-amidation,” Annu. Rev. Neuroscience, 15:57-85, 1992a.

Eizirik, Korbutt, Hellerström, “Prolonged exposure of human pancreatic islets to high glucose concentrations in vitro impairs the β-cell function,” J. Clin. Invest., 90:1263-1268, 1992.

EPA 0 273 085

EPA 0 320 308

EPA 0 329 822

Epstein, et al., “Expression of yeast hexokinase in pancreatic beta-cells of transgenic mice reduces blood glucose, enhances insulin secretion, and decreases diabetes,” Proc. Nat'l Acad. Sci. USA, 89:12038-12042, 1992.

Ercolani et al., “Isolation and complete sequence of a functional human glyceraldehyde-3-phosphate dehydrogenase gene,” J. Biol. Chem., 263;15335-15341, 1988.

Eshkol, “Mammalian cell-derived recombinant human growth hormone: Pharmacology, metabolism and clinical results,” Hormone Research, 37:1-3, 1992.

Fanciulli et al., Glycolysis and growth rate in normal and in hexokinase-transfected NIH-3T3 cells,” Oncology Res., 6(9): 405-409, 1994.

Fechheimer et al., “Transfection of mammalian cells with plasmid DNA by scrape loading and sonication loading,” Proc. Nat'l Acad. Sci. USA, 84:8463-8467, 1987.

Felgner, et al., Arch. Bioch. Biophys. 182:282-94, 1977.

Ferber et al., “Transfection of rat insulinoma cells with GLUT-2 confers both low and high affinity glucose-stimulated insulin release: relationship to glucokinase activity,” J. Biol. Chem., 269:11523-11529, 1994.

Ferber, et al., “GLUT-2 Gene Transfer into Insulinoma Cells Confers Both Low and High Affinity Glucose-stimulated insulin Release”, J. Biological Chemistry, 269:12523-12529, 1994.

Ferkol et al., FASEB J., 7:1081-1091, 1993.

Fiedorek et al., “Selective expression of the insulin I gene in rat insulinoma-derived cell lines,” Mol. Endocrin., 4:990-999, 1990.

Fiek et al., “Evidence for identity between hexokinase-binding protein and the mitochondrial porin in the outer membrane of rat liver mitochondria,” Biochem. Biophys. Acta, 688:429-440, 1982.

Forster and Symons, “Self-cleavage of plus and minus RNAs of a virusoid and a structural model for the active sites,” Cell, 49:211-220, 1987.

Fort et al., “Various rat adult tissues express only one major species from the glyceraldehyde-3-phosphate-dehydrogenase multigenic family,” Nucleic Acids Research, 13:1431-1442, 1985.

Fraley et al., “Entrapment of a bacterial plasmid in phospholipid vesicles: Potential for gene transfer,” Proc. Nat'l Acad. Sci. USA, 76:3348-3352, 1979.

Fricker, “Carboxypeptidase E,” Ann. Rev. Physiology, 50:309-321, 1988.

Friedmann, “Progress toward human gene therapy,” Science, 244:1275-1281, 1989.

Fritschy et al., Diabetes, 40:37, 1991.

Frohman, In: PCR Protocols: A guide to methods and applications, Academic Press, N.Y., 1990.

Frougel et al., “Familial hyperglycemia due to mutations in glucokinase,” N. Engl. J Med., 328:105-112, 1993.

Gasa et al., J. Biol. Chem., 275(34):26396-26403, 2000.

Gasa, Trinh, Yu, Wilkie, Newgard, “Manipulation of inositol phosphate levels by overexpression of phospholipase C isoforms and/or heterotrimeric G protein subunits is without effect on insulin secretion,” Diabetes, 48:1035-1044, 1999.

Gazdar et al. “Continuous, clonal, insulin- and somatostatin-secreting cell lines established from a transplantable rat islet cell tumor,” Proc. Nat'l Acad. Sci. USA, 77:3519-3523, 1980.

GB Application No. 2 202 328

Gelb et al., “Targeting of hexokinase 1 to liver the hepatoma mitochondria,” Proc. Nat'l Acad. Sci. USA, 89:202-206, January, 1992.

Gembal, Gilon, Henquin, “Evidence that glucose can control insulin release independently from its action on ATP-sensitive K⁺ channels in mouse B cells.” J. Clin. Invest., 89:128008-1295, 1992.

Gerlach et al., “Construction of a plant disease resistance gene from the satellite RNA of tobacco ringspot virus,” Nature (London), 328:802-805, 1987.

German et al., “The insulin and islet amyloid polypeptide genes contain similar cell-specific promoter elements that bind identical β-cell nuclear complexes,” Mol. Cell. Biol., 12:1777-1788, 1992.

Ghosh and Bachhawat, “Targeting of liposomes to hepatocytes,” In: Wu and Wu (ed.) Liver diseases, targeted diagnosis and therapy using specific receptors and ligands. New York: Marcel Dekker, pp. 87-104, 1991.

Ghosh-Choudhury et al., “Protein IX, a minor component of the human adenovirus capsid, is essential for the packaging of full-length genomes,” EMBO J., 6:1733-1739, 1987.

Giddings et al., “Effects of glucose on proinsulin messenger RNA in rats in vivo,” Diabetes, 31:624-629, 1982.

Gomez-Foix et al., “Adenovirus-mediated transfer of the muscle glycogen phosphorylase gene into hepatocytes confers altered regulation of glycogen,” J. Biol. Chem., 267:25129-25134, 1992.

Gopal, “Gene transfer method for transient gene expression, stable transfection, and cotransfection of suspension cell cultures,” Mol. Cell Biol., 5:1188-1190, 1985.

Gossen et al., “Tight control of gene expression in mammalian cells by tetracycline-responsive promoters,” Proc. Nat'l Acad. Sci. USA, 89:5547-5551, 1992.

Graham and Prevec, “Adenovirus-based expression vectors and recombinant vaccines,” Biotechnology, 20:363-390, 1992.

Graham and Prevec, “Manipulation of adenovirus vector,” In: Murray (ed.), methods in molecular biology: gene transfer and expression protocol, Clifton, N.J.: Humana Press, 7:109-128, 1991.

Graham and van der Eb, “A new technique for the assay of infectivity of human adenovirus 5 DNA”, Virology, 52:456-467, 1973.

Graham et al., “Characteristics of a human cell line transformed by DNA from human adenovirus type 5”, J. Gen. Virol., 36:59-72, 1977.

Grampp et al., “Use of regulated secretion in protein production from animal cells: An overview,” Advances in Biochemical Engineering, 46:35-62, 1992.

Gross et al., “Partial diversion of a mutant proinsulin (B10 aspartic acid) from the regulated to the constitutive secretory pathway in transfected AtT-20 cells,” P.N.A.S., 86:4107-4111, 1989.

Grunhaus and Horwitz, “Adenovirus as cloning vector,” Seminar in Virology, 3:237-252, 1992.

Gulbis and Galand, “Immunodetection of the p21-ras products in human normal and preneoplastic tissues and solid tumors: a review,” Hum. Pathol., 24(12):1271-1285, 1993.

Hakes et al., “Isolation of two complementary deoxyribonucleic acid clones from a rat insulinoma cell line based on similarities to Kex2 and furin sequences and the specific localization of each transcript to endocrine and neuroendocrine tissues in rat,” Endocrinology, 129:3053-3063, 1991.

Halban et al, “Abnormal glucose metabolism accompanies failure of glucose to stimulate insulin release from a pancreatic cell line (RINm5f),” Biochem. J., 212:439-443, 1983.

Halban et al., “High-performance liquid chromatography (HPLC): a rapid, flexible and sensitive method for separating islet proinsulin and insulin,” Daibetologia, 29:893-896, 1986.

Halban et al., “Intracellular degradation of insulin stores by rat pancreatic islets in vitro: An alternative pathway for homeostasis of pancreatic insulin content,” J. Biol Chem., 255:6003-6006, 1980.

Halban, “Structural domains and molecular lifestyles of insulin and its precursors in the pancreatic beta cell,” Daibetologia, 34:767-778, 1991.

Harland and Weintraub, “Translation of mammalian mRNA injected into Xenopus oocytes is specifically inhibited by antisense RNA,” J. Cell Biol., 101: 1094-1099, 1985.

Haseloff and Gerlach, “Simple RNA enzymes with new and highly specific endoribonuclease activities,” Nature, 334:585-591, 1988.

Heartlein et al., “Long-term production and delivery of human growth hormone in vivo,” P.N.A.S., 91:10967-10971, 1994.

Heinrich et al., “Pre-glucagon messenger ribonucleic acid: Nucleotide and encoded amino acid sequences of the rat pancreatic complementary deoxyribonucleic acid., Endocrinology, 115:2176-2181, 1984.

Hermonat and Muzycska, “Use of adenoassociated virus as a mammalian DNA cloning vector: Transduction of neomycin resistance into mammalian tissue culture cells,” Proc. Nat'l Acad. Sci. USA, 81:6466-6470, 1984.

Hersdorffer et al., “Efficient gene transfer in live mice using a unique retroviral packaging line,” DNA Cell Biol., 9:713-723, 1990.

Herz and Gerard, “Adenovirus-mediated transfer of low density lipoprotein receptor gene acutely accelerates cholesterol clearance in normal mice,” Proc. Nat'l Acad. Sci. USA 90:2812-2816, 1993.

Hohmeier et al., “Isolation of INS-1-Derived Cell Lines with Robust K_{ATP} Channel-Dependent and-Independent Glucose-Stimulated Insulin Secretion Diabetes, 49: 424, 2000.

Hohmeier et al., “Regulation of insulin secretion from novel engineered insulinoma cell lines,” Diabetes, 46:958-967, 1997.

Hoppener et al., “Molecular physiology of the islet amyloid polypeptide (IAAP)/amylin gene in man, rat, and transgenic mice,” J. Cell. Biochem., 55S:39-53, 1994.

Horwich et al. “Synthesis of hepadenovirus particles that contain replication-defective duck hepatitis B virus genomes in cultured HuH7 cells,” J. Virol., 64:642-650, 1990.

Hosokawa, et al., “Upregulated hexokinase activity in isolated islets from diabetic 90% pancreatectomized rats.” Diabetes 44:1328-1333, 1995.

Hubank and Schatz, Nucleic Acids Research, 22(25):5640-5648, 1994.

Hughes et al., “Engineering of Glucose-stimulated Insulin Secretion and Biosynthesis in Non-islet Cells”, Proc. Nat'l Acad. Sci. USA, 89:688-692, 1992.

Hughes et al., “Expression of normal and novel glucokinase mRNA's in anterior pituitary and islet cells,” J. Biol. Chem., 266:4521-4530, 1991.

Hughes et al., “Transfection of AtT-20ᵢₙₛ Cells with GLUT-2 but Not GLUT-1 Confers Glucose-stimulated Insulin Secretion”, The Journal of Biological Chemistry, 268:15205-15212, 1993.

Hughes et al., “Transfection of AtT-20ins cells with GLUT-2 but not GLUT-1 confers glucose-stimulated insulin secretion: relationship to glucose metabolism,” J.B.C., 268:15205-15212, 1993.

Huypens et al., Daibetologia 43(8):1012-9, 2000.

Inagaki et al., “Reconstitution of IKATP: An inward rectifier subunit plus the sulfonylurea receptor,” Science, 270:1166-1170, 1995.

Innis et al., “DNA sequencing with Thermus aquaticus DNA polymerase and direct sequencing of polymerase chain reaction-amplified DNA,” Proc. Nat'l Acad. Sci. USA. 85(24):9436-9440, 1988.

Isaksson et al., “Mode of action of pituitary growth hormone on target cells,” Ann. Rev. Physiol., 47:483-499, 1985.

Ishibashi et al., “Hypercholesterolemia in low density lipoprotein receptor knockout mice and its reversal by adenovirus-mediated gene delivery,” J. Clin. Invest, 92:883-893, 1993.

Ishihara et al., “Overexpression of hexokinase I but not GLUT1 glucose transporter alters concentration dependence of glucose-stimulated insulin secretion in pancreatic beta-cell line MIN6,” J.Biol.Chem., 269:3081-3087, 1994.

Jang et al., “Initiation of protein synthesis by internal entry of ribosomes into the 5′ nontranslated region of encephalomyocarditis virus RNA in vivo,” J. Virology, 63:1651-1660, 1989.

Johnson et al., “Underexpression of Beta cell high Kₘ glucose transporters in noninsulin-dependent diabetes,” Science, 250:546-549, 1990.

Jones and Shenk, “Isolation of deletion and substitution mutants of adenovirus type 5,” Cell, 13:181-188, 1978.

Jonsson et al., “Insulin-promoter-factor 1 is required for pancreas development in mice,” Nature, 371:606-609, 1994.

Jonsson, Carlsson, Edlund, Edlund, Nature, 371:606-609, 1994.

Joyce, “RNA evolution and the origins of life,” Nature, 338:217-244, 1989.

Kabir and Wilson, “Mitochondrial Hexokinase in Brain: Coexistence of Forms Differing in Sensitivity to Solubilization by Glucose-6-Phosphate on the Same Mitochondria,” Arch. Biochem,. Biophys., 310(2):410-416, 1994.

Kaneda et al., “Increased expression of DNA cointroduced with nuclear protein in adult rat liver,” Science, 243:375-378, 1989.

Kang et al., Science, 240:1034-1036, 1988.

Karlsson et al., “A mutational analysis of the insulin gene transcription control region: expression in β-cells is dependent on two related sequences within the enhancer,” Proc. Nat'l Acad. Sci. USA, 84:8819-8823, 1987.

Karlsson et al., “Individual protein-binding domains of the insulin gene enhancer positively activate β-cell-specific transcription,” Mol. Cell. Biol., 9:823-827, 1989.

Karlsson et al., EMBO J., 5:2377-2385, 1986.

Kato et al., “Expression of hepatitis β virus surface antigen in adult rat liver,” J. Biol. Chem., 266:3361-3364, 1991.

Kaufman et al., “Construction of a modular dihydrofolate reductase cDNA gene: Analysis of signals utilized for efficient expression,” Mol. Cell. Biology, 2:1304-1319, 1982.

Keller et al., “Insulin prophylaxis in individuals at high risk for the development of type I diabetes mellitus,” Lancet., 341:927-928, 1993.

Kennedy et al., “The minisatellite in the diabetes susceptibility locus IDDM2 regulates insulin transcription,” Nature Genetics, 9:293-298, 1995.

Khatoon et al., Ann. Neurology, 26, 210-219, 1989.

Khatoon et al., Ann. of Neurology, 26, 210-219, 1989.

Kim & Cech, “Three dimensional model of the active site of the self-splicing rRNA precursor or Tetrahymena,” Proc. Nat'l Acad. Sci. USA, 84:8788-8792, 1987.

King et al., J. Biol. Chem., 269, 10210-10218, 1989.

Klein et al., “High-velocity microprojectiles for delivering nucleic acids into living cells,” Nature, 327:70-73, 1987.

Kmiec, E. B., Cole, A., and Holloman, W. K. 1994. The REC2 gene encodes the homologous pairing protein of Ustilago maydis. Mol. Cell. Biol. 14: 7163-7172.

Knaack et al., “Clonal Insulinoma Cell Line That Stably Maintains Correct Glucose Responsiveness,” Diabetes, 43:1413-1417, December, 1994.

Kohler & Milstein, Nature 256:495, 1975.

Kohler et al., Methods Enzymol., 178:3, 1989.

Kreier et al., Infection, Resistance and Immunity, Harper and Row, New York, 1991.

Kreig and Melton, “Functional messenger RNA's are produced by SP6 in vitro transcription of cloned cDNA's,” N.A.R., 12:7057-7070, 1984.

Kreymann et al., “Glucagon-like peptide-1 7-36: a physiological incretion in man,” Lancet, 2:1300-1303, 1987.

Kruse et al., “An endocrine-specific element is an integral component of an exocrine-specific pancreatic enhancer,” Genes and Dev., 7:774-786, 1993.

Kuwajima M, Newgard C B, Foster D W, McGarry J D. The glucose phosphorylating capacity of liver as measured by three independent assays: implication for the mechanism of hepatic glycogen synthesis. J. Biol. Chem., 261: 8849-53, 1986.

Kwoh et al., “Transcription-based amplification system and detection of amplified human immunodeficiency virus type 1 with a bead-based sandwich hybridization format, Proc Nat'l Acad Sci USA 86(4):1173-1177, 1989.

Lacy et al., Science, 254:1782-1784, 1991.

Larsson and Litwin, “The growth of polio virus in human diploid fibroblast grown with cellulose microcarries in suspension culture,” Dev. Biol. Standard., 66:385-390, 1987.

Le Gal La Salle et al., “An adenovirus vector for gene transfer into neurons and glia in the brain,” Science, 259:988-990, 1993.

Leffert et al., “Rat amylin: Cloning and tissue-specific expression in pancreatic islets,” Proc. Nat'l Acad. Sci. USA, 86: 3127-3130, 1989.

Lenert et al., Science, 248:1639-1643, 1990.

Leonard et al., “Characterization of Somatostatin Transactivating Factor-1, a Novel Homeobox Factor That Stimulates Somatostatin Expression in Pancreatic Islet Cells,” Mol Endocrinol, 7:1275-1283, 1993.

Levrero et al., “Defective and nondefective adenovirus vectors for expressing foreign genes in vitro and in vivo,” Gene, 101:195-202, 1991.

Li et al., “Effect of disruption of actin filaments by Clostridium botulinum C2 toxin on insulin secretion in HIT-T15 cells and pancreatic islets,” M.B.C., 5:1199-1213, 1994.

Liang et al., “Effects of alternate RNA splicing on glucokinase isoform activities in the pancreatic islet, liver, and anterior pituitary,” J.B.C., 266:6999-7007, 1991.

Liang et al., “Enhanced and Switchable Expression Systems for Gene-Transfer,” J. Cell. Biochem. Supplement 21A:379, 1995.

Liang Y, Bai G, Doliba N, Buettger C, Wang L Q, Berner D K, Matschinsky F M, “Glucose metabolism and insulin release in mouse beta-HC9 cells, as model for wild-type pancreatic beta-cells.” Am J. Phys. 33: E846-E857, 1996.

Liang, et al. “Glucose regulates glucokinase activity in cultured islets from rat pancreas.” J. Biol. Chem. 265:16863-16866, 1990.

Lieber, A. and Strauss, M. “Selection of efficient cleavage sites in target RNAs by using a ribozyme expression library.” Mol. Cell. Biol., 15: 540-551, 1995.

Lin et al., “Pit-1-dependent pituitary cell proliferation involves activation of the growth hormone releasing factor receptor gene,” Nature, 360:765-768, 1992.

Linden et al., “Pore protein and the hexokinase-binding protein from the outer membrane of rat liver mitochondria are identical,” FEBS Lett., 141:189-192, 1982.

Macejak and Sarnow, “Internal initiation of translation mediated by the 5′ leader of a cellular mRNA,” Nature, 353:90-94, 1991.

Madsen, et al., Proc. Nat'l Acad. Sci. U.S.A., 85:6652-6656, 1988.

Mann et al., “Construction of a retrovirus packaging mutant and its use to produce helper-free defective retrovirus,” Cell, 33:153-159, 1983.

Marchetti et al., “Pulsatile insulin secretion from isolated human pancreatic islets,” Diabetes, 43:827-830, 1994.

Marie et al., “The pyruvate kinase gene is a model for studies of glucose-dependent regulation of gene expression in the endocrine pancreatic Beta-cell type,” J.B.C., 268:23881-23890, 1993.

Markowitz et al., “A safe packaging line for gene transfer: Separating viral genes on two different plasmids,” J. Virol., 62:1120-1124, 1988.

Mayo, “Molecular cloning and expression of a pituitary-specific receptor for growth hormone-releasing hormone,” Mol. Endocrinol., 1734-1744, 1992.

Meglasson and Matschinsky, “Pancreatic islet glucose metabolism and regulation of insulin secretion,” Diabetes Metab. Rev., 2:163-214, 1986.

Michel and Westhof, “Modeling of the three-dimensional architecture of group I catalytic introns based on comparative sequence analysis,” J. Mol. Biol., 216:585-610, 1990.

Milgram et al., “Expression of individual forms of peptidylglycine alpha-amidating monooxygenase in AtT-20 cells: Endoproteolytic processing and routing to secretory granules,” J.Chem.Biol., 117:717-728, 1992.

Miller et al., “IDX-1: a new homeodomain transcription factor expressed in rat pancreatic islets and duodenum that transactivates the somatostatin gene,” EMBO J, 13:1145-1156, 1994.

Mirmira et al., J. Biol. Chem., 275:14743-14751, 2000.

Miyazaki et al., “Establishment of a pancreatic Beta-cell line that retains glucose-inducible insulin secretion: special reference to expression of glucose transporter isoforms,” Endocrinology, 127:126-132, 1990.

Mizrahi, “Production of human interferons: An overview,” Process Biochem., (August):9-12, 1983.

Mojsov et al., “Both amidated and nonamidated forms of glucagon-like peptide 1 are synthesized in the rat intestine and the pancreas,” J.B.C., 265:8001-8008, 1990.

Mojsov et al., “Preglucagon gene expression in the pancreas and intestine diversifies at the level of post-translational processing,” J.B.C., 261:11880-11889, 1986.

Monia, “Evaluation of 2′-modified oligonucleotides containing 2′-deoxy gaps as antisense inhibitors,” J. Biol. Chem., 268:14541-14521, 1993.

Moore et al., “Expressing a human proinsulin cDNA in a mouse ACTH-secreting cell. Intracellular storage, proteolytic processing, and secretion on stimulation,” Cell, 35:531-538, 1983.

Moore et al., “Secretory protein targeting in a pituitary cell line: Differential transport of foreign secretory proteins to distinct secretory pathways,” J.C.B., 101:1773-1781, 1985.

Muir et al., “Antigen specific immunotherapy. Oral tolerance and subcutaneous immunization in the treatment of insulin-dependent diabetes,” Diabetes. Metab. Rev., 9:279-287, 1993.

Muir et al., “Insulin immunization of nonobese diabetic mice induces a protective insulitis characterized by diminished intraislet interferon-gamma transcription,” J. Clin. Invest., 95:628-634, 1995.

Mulligan et al., “Expression of a bacterial gene in mammalian cells,” Science, 209:1422-1427, 1980.

Mulligan et al., “Selection for animal cells that express the Escherichia coli gene coding xanthine-guanine phosphoribosyltransferase,” Proc. Nat'l Acad. Sci. USA, 78:2072-2076, 1981.

Mulligan, “The Basic Science of Gene Therapy,” Science, 260:926-932, 1993.

Munson and Pollard, Anal. Biochem., 107:220, 1980.

Naya, Huang, Qiu, Mutoh, DeMayo, Leiter, Tsai, Genes Dev., 11:2323-2334, 1997.

Newgard et al., “Glucokinase and glucose transporter expression in liver and islets: implications for control of glucose homeostasis,” Biochem. Soc. Trans., 18:851-853, 1990.

Newgard, “Cellular engineering and gene therapy strategies for insulin replacement in diabetes,” Diabetes, 43:341-350, 1994.

Newgard, “Regulatory role of glucose transport and phosphorylation in pancreatic islet β-cells,” Diabetes Reviews, 4:191-206, 1996.

Newgard, et al., “Metabolic coupling factors in pancreatic Beta-cell signal transduction,” Ann. Rev. Biochem., 64:689-719, 1995.

Nicolas and Rubinstein, “Retroviral vectors,” In: Vectors: A survey of molecular cloning vectors and their uses, Rodriguez and Denhardt (eds.), Stoneham: Butterworth, pp. 494-513, 1988.

Nicolau and Sene, “Liposome-mediated DNA transfer in eukaryotic cells,” Biochem. Biophys. Acta, 721:185-190, 1982.

Nicolau et al., “Liposomes as carriers for in vivo gene transfer and expression,” Methods Enzymol., 149:157-176, 1987.

Nilsson and Mosbach, “Immobilized animal cells,” Dev. Biol. Standard., 66:183-193, 1987.

Noda, Komatsu, Sharp, “The beta-HC-9 pancreatic beta-cell line preserves the characteristics of progenitor mouse islets,” Diabetes, 45:1766-1773, 1996.

Noel, Antinozzi, McGarry, Newgard, “Engineering of glycerol-stimulated insulin secretion in islet beta cells,” J. Biol. Chem., 272:18621-18627, 1997.

Offield, Jetton, Labosky, Ray, Stein, Magnuson, Hogan, Wright, Development, 122:983-995, 1996.

Ogawa et al., “Loss of glucose-induced insulin secretion and GLUT-2 expression in transplanted Beta-cells,” Diabetes, 44:75-79, 1995.

Ogawa et al., “Roles of insulin resistance and β-cell dysfunction in dexamethasone-induced diabetes,” J. Clin. Invest., 90:497-502, 1992

Ohagi et al., “Identification and analysis of the gene encoding PC2, a prohormone convertase expressed in neuroendocrine tissues,” P.N.A.S., 89:4977-4981, 1992.

Ohara et al., “One-sided polymerase chain reaction: the amplification of cDNA,” Proc. Nat'l Acad. Sci. USA. 86(15):5673-5677, 1989.

Ohlsson et al., “IPF1, a homeodomain-containing transactivator of the insulin gene,” EMBO J., 12:4251-4259, 1993.

Orci et al., “Evidence that down-regulation of Beta-cell glucose transporters in non-insulin-dependent diabetes may be the cause of diabetic hyperglycemia,” P.N.A.S., 87:9953-9957, 1990.

Orskov et al., “Biological effects and metabolic rates of glucagonlike peptide-1 7-36 amide and glucagonlike peptide-1 7-37 in healthy subjects are indistinguishable,” Diabetes, 42:658-661, 1993.

Orskov et al., “Complete sequence of glucagon-like peptide-1 from human and pig small intestine,” J. Biol. Chem., 264:12826-12829, 1989.

O'Shannessy et al, J. Immun. Meth., 99, 153-161, 1987.

O'Shea and Sun, Diabetes 35:943-946, 1986.

Ouafik et al., “Developmental regulation of peptidylglycine alpha-amidating monooxygenase (PAM) in rat heart atrium and ventricle,” J.B.C., 264:5839-5845, 1989.

Ouafik et al., “The multifunctional peptidylglycine alpha-amidating monooxygenase gene: Exon/intron organization of catalytic, processing, and routing domains,” Molecular Endocrinology, 6:1571-1584, 1992.

Owens and Haley, J. Biol. Chem., 259:14843-14848, 1987.

Owerbach and Aagaard, “Analysis of a 1963-bp polymorphic region flanking the human insulin gene,” GENE, 32:475-479, 1984.

Palmer et al., “Genetically modified skin fibroblasts persist long after transplantation but gradually inactivate introduced genes,” Proc. Nat'l Acad. Sci. USA, 88:1330-1334, 1991.

Palmer et al., “Production of human Factor IX in animals by genetically modified skin fibroblasts: Potential therapy for hemophilia B,” Blood, 73:438-445, 1989.

Palukaitis et al., “Characterization of a viroid associated with avocado sunblotch disease,” Virology, 99:145-151, 1979.

Paskind et al., “Dependence of moloney murine leukemia virus production on cell growth,” Virology, 67:242-243, 1975.

Pavlakis and Hamer, “Regulation of a metallothionein-growth hormone hybrid gene in bovine papilloma virus,” P.N.A.S. 80:397-401, 1983.

PCT Application No. PCT/US87/00880.

PCT Application No. PCT/US89/01025.

Peers et al., “Insulin Expression in Pancreatic Islet Cells Relies on Cooperative Interactions between the Helix Loop Helix Factor E47 and the Homeobox Factor STF-1,” Mol Endocrin, 8:1798-1806, 1994.

Pelletier and Sonenberg, “Internal initiation of translation of eukaryotic mRNA directed by a sequence derived from poliovirus RNA,” Nature, 334:320-325, 1988.

Perales et al., Proc. Nat'l Acad. Sci. USA, 91:4086-4090, 1994.

Perriman et al., “Extended target-site specificity for a hammerhead ribozyme,” Gene, 113:157-163, 1992.

Perrotta and Been, Biochem. 31:16, 1990.

Petricciani, “Should continuous cell lines be used as substrates for biological products?,” Dev. Biol. Standard., 66:3-12, 1985.

Philippe et al., “Multipotential phenotypic expression of genes encoding peptide hormones in rat insulinoma lines,” J. Clin. Invest., 79:351-358, 1987.

Phillips et al., In: Large Scale Mammalian Cell Culture (Feder, J. and Tolbert, W. R., eds.), Academic Press, Orlando, Fla., U.S.A., 1985.

Pieber et al., “Direct plasma radioimmunoassay for rat amylin-(1-37): concentrations with acquired and genetic obesity,” Am. J. Physiol., 267:E156-E164, 1994.

Polakis and Wilson, “An Intact Hydrophobic N-Terminal Sequence Is Critical for Binding of Rat Brain Hexokinase to Mitochondria,” Arch. Biochem. Biophys., 236(1):328-337, 1985.

Pontoglio et al., “Defective insulin secretion in hepatocyte nuclear factor 1α-deficient mice,” J. Clin. Invest., 101:2215-2222, 1998.

Potter and Haley, Meth. Enzymol., 91:613-633, 1983.

Potter et al., “Enhancer-dependent expression of human k immunoglobulin genes introduced into mouse pre-B lymphocytes by electroporation,” Proc. Nat'l Acad. Sci. USA, 81:7161-7165, 1984.

Powell et al, “Efficient targeting to storage granules of human proinsulins with altered propeptide domain,” J. Chem. Biol., 106:1843-1851, 1988.

Prentki, “New insight into pancreatic beta-cell metabolic signaling.” Eur. J. Endocrinol., 134:272-286, 1996.

Prody et al., “Autolytic processing of dimeric plant virus satellite RNA.” Science, 231:1577-1580, 1986.

Quaade et al., “Analysis of the protein products encoded by variant glucokinase transcripts via expression in bacteria,” FEBS Lett, 280:47-52, 1991.

Racher et al., Biotechnology Techniques, 9:169-174, 1995.

Radvanyi et al., “Pancreatic beta bells cultured from individual preneoplastic foci in a multistage tumorigenesis pathway: a potentially general technique for isolating physiologically representative cell lines,” Molec. and Cell. Biol., 13(7):4223-4232, 1993.

Ragot et al., “Efficient adenovirus-mediated transfer of a human minidystrophin gene to skeletal muscle of mdx mice,” Nature, 361:647-650, 1993.

Reinhold-Hurek and Shub, “Self-splicing introns in tRNA genes of widely divergent bacteria,” Nature, 357:173-176, 1992.

Renan, “Cancer genes: current status, future prospects, and applicants in radiotherapy/oncology,” Radiother. Oncol., 19:197-218, 1990.

Rhodes and Alarcon, “What beta-cell defect could lead to hyperproinsulinemia in NIDDM?,” Diabetes, 43:511-517, 1994.

Rhodes and Halban, “The intracellular handling of insulin-related peptides in isolated pancreatic islets,” Biochem. J., 251:23-30, 1988.

Rich et al., “Development and analysis of recombinant adenoviruses for gene therapy of cystic fibrosis,” Hum. Gene Ther., 4:461-476, 1993.

Ridgeway, “Mammalian expression vectors,” In: Rodriguez R L, Denhardt D T, ed. Vectors: A survey of molecular cloning vectors and their uses. Stoneham: Butterworth, pp. 467-492, 1988.

Rippe et al., “DNA-mediated gene transfer into adult rat hepatocytes in primary culture,” Mol. Cell Biol., 10:689-695, 1990.

Roche et al., “Induction by glucose of genes encoding for glycolytic enzymes in a pancreatic beta cell line (INS-1),” J. Biol. Chem., 272:3091-3098, 1997.

Rosenfeld et al., “Adenovirus-mediated transfer of a recombinant a1-antitrypsin gene to the lung epithelium in vivo,” Science, 252:431-434, 1991.

Rosenfeld et al., “In vivo transfer of the human cystic fibrosis transmembrane conductance regulator gene to the airway epithelium,” Cell, 68:143-155, 1992.

Rouille et al., “Differential processing of proglucagon by the subtilisin-like prohormone convertases PC2 and PC3 to generate either glucagon or glucagon-like peptide,” J.B.C., 270:26488-26496, 1995.

Rouille et al., “Proglucagon is processed to glucagon by prohormone convertase PC2 in alphaTC1-6 cells,” P.N.A. S., 91:3242-3246, 1994.

Roux et al., “A versatile and potentially general approach to the targeting of specific cell types by retroviruses: Application to the infection of human cells by means of major histocompatibility complex class I and class II antigens by mouse ecotropic murine leukemia virus-derived viruses,” Proc. Nat'l Acad. Sci. USA, 86:9079-9083, 1989.

Rubin, Ferguson, Holloman, “Structure of REC2, a recombinational repair gene of Ustilago maydis, and its function in homologous recombination between plasmid and chromosomal sequences,” Mol. Cell. Biol., 14:6287-6296, 1994.

Sadelain et al., “Prevention of type I diabetes in NOD mice by adjuvant immunotherapy,” Diabetes, 39:583-589, 1990.

Sambanis et al., “A model of secretory protein trafficking in recombinant AtT-20 cells,” Biotechnology and Bioengineering, 36:280-295, 1991.

Sambanis et al., “Use of regulated secretion in protein production from animal cells: An evaluation with the AtT-20 model cell line,” Biotechnology and Bioengineering, 35:771-780, 1990.

Sambrook, Fritsch, Maniatis, In: Molecular Cloning: A Laboratory Manual 2 rev.ed., Cold Spring Harbor: Cold Spring Harbor Laboratory Press, 1(7,7):19-17.29, 1989.

Sander and German, J. Mol. Med., 75:327-340, 1997.

Sander, Kalamaras, German, Keystone Symposium on Vertebrate Development, Steamboat Springs, Colo., Apr. 3-8, 1998

Sander, Neubuser, Kalamaras, Ee, Martin, German, Genes Dev., 11:1662-1673, 1997.

Sanke et al., “An islet amyloid peptide is derived from an 89-amino acid precursor by proteolytic processing,” J.B.C., 263:17243-17246, 1988.

Santerre et al., “Insulin synthesis in a clonal cell line of simian virus 40-transformed hamster pancreatic beta-cells,” P.N.A.S., 78:4339-4343, 1981.

Sarver, et al, “Ribozymes as potential anti-HIV-1 therapeutic agents,” Science, 247:1222-1225, 1990.

Sasso et al., J. Immunol., 142:2778-2783, 1989.

Sato et al, Proc. Nat'l Acad. Sci. U.S.A. 48:1184-1190, 1962.

Sauer, “Manipulation of transgenes by site-specific recombination: Use of Cre recombinase,” Methods in Enzymology, 225:890-900, 1993.

Scanlon et al., “Ribozyme-mediated cleavages of c-fos mRNA reduce gene expression of DNA synthesis enzymes and metallothionein,” Proc Natl Acad Sci USA, 88:10591-10595, 1991.

Scharfmann et al., “Long-term in vivo expression of retrovirus-mediated gene transfer in mouse fibroblast implants,” P.N.A.S. USA, 88:4626-4630, 1991.

Scharp et al., “Protection of encapsulated human islets implanted without immunosuppression in patients with Type I or Type II diabetes and in nondiabetic control subjects,” Diabetes, 43:1167-1170, 1994.

Schmidt et al., “Synthesis and targeting of insulin-like growth factor-1 to the hormone storage granules in an endocrine cell line,” J.B.C., 269:27115-27124, 1994.

Schnedl et al., “STZ transport and cytotoxicity: Specific enhancement in GLUT-2-expressing cells,” Diabetes, 43:1326-1333, 1994.

Schnedl, Ferber, Johnson, Newgard, “STZ transport and cytotoxicity, specific enhancement in GLUT2-expressing cells,” Diabetes, 13:1326-1333, 1994.

Schnetzler et al., “Adaptation to supraphysiological levels of insulin gene expression in transgenic mice: Evidence for the importance of posttranscriptional regulation,” J. Clin. Invest., 92:272-280, 1994.

Schwab et al., “Complete amino acid sequence of rat brain hexokinase, deduced from the cloned cDNA, and proposed structure of a mammalian hexokinase,” Proc. Nat'l Acad. Sci. USA, 86:2563-2567, 1989.

Seeburg, “The human growth hormone gene family: Nucleotide sequences show recent divergence and predict a new polypeptide hormone,” DNA, 1;239-249, 1982.

Selden et al., “Implantation of genetically engineered fibroblasts into mice: Implications for gene therapy,” Science, 236:714-718, 1987.

Sevarino et al., “Cell-specific processing of preprosomatostatin in cultured neuroendocrine cells,” J.B.C., 262:4987-4993, 1987.

Sevarino et al., “Thyrotropin releasing hormone (TRH) precursor processing,” J.B.C., 264:21529-21535, 1989.

Shehadeh et al., “Effect of adjuvant therapy on development of diabetes in mouse and man,” Lancet., 343:706-707, 1994.

Shimizu et al., “Control of glucose phosphorylation and glucose usage in clonal insulinoma lines,” Diabetes, 37:563-568, 1988.

Shorki et al., J. Immunol., 146:936-940, 1991.

Silvermann et al., J. Clin. Invest., 96:417-426, 1995.

Sioud et al., “Preformed ribozyme destroys tumour necrosis factor mRNA in human cells,” J. Mol. Biol., 223:831-835, 1992.

Sizonenko and Halban, “Differential rates of conversion of rat proinsulins I and II:

Evidence for slow cleavage at the B-chain/C-chain junction of proinsulin II,” Biochemistry J., 278:621-625, 1991.

Skelly, Bollheimer, Wicksteed, Corkey, Rhodes, “A distinct difference in the metabolic stimulus-response coupling pathways for regulating proinsulin biosynthesis and insulin secretion that lies at the level of a requirement for fatty acyl moieties,” Biochem J., 331:553-561,1993.

Smeekens and Steiner, “Identification of a human insulinoma cDNA encoding a novel mammalian protein structurally related to the yeast dibasic processing protease Kex2,” J.B.C., 265:2997-3000, 1990.

Smith, et al., Arch. Bioch. Biophys. 287:359-366, 1991.

Sosa-Pineda, Chowdhury, Torres, Oliver, Gruss, Nature, 386:199-402, 1997.

Steiner et al., “The new enzymology of precursor processing endoproteases,” J.B.C., 267:23435-23438, 1992.

Stoffers et al., “Alternative mRNA splicing generates multiple forms of peptidyl-glycine alpha-amidating monooxygenase in rat atrium,” P.N.A.S., 86:735-739, 1989.

Stoffers et al., “Characterization of novel mRNA's encoding enzymes involved in peptide alpha-amidation,” J.B.C., 266:1701-1707, 1991.

St-Onge, Sosa-Pineda, Chowdhury, Mansouri, Gruss, Nature, 387:406-409, 1997.

Stossel, “The Molecular Basis of Blood Diseases,” Chapter 14, pp. 499-533, W. B. Saunders Co., Philadelphia, Pa., 1987.

Stratford-Perricaudet and Perricaudet, “Gene transfer into animals: the promise of adenovirus,” p. 51-61, In: Human Gene Transfer, Eds, O. Cohen-Haguenauer and M. Boiron, Editions John Libbey Eurotext, France, 1991.

Stratford-Perricaudet et al., “Evaluation of the transfer and expression in mice of an enzyme-encoding gene using a human adenovirus vector,” Hum. Gene Ther., 1:241-256, 1990.

Subramani et al., “Expression of the mouse dihydrofolate reductase complementary deoxyribonucleic acid in simian virus 40 vectors,” Mol. Cell. Biol., 1:854-864, 1981.

Sullivan et al., Science 252:718-721, 1991.

Sussel, Kalamaras, Hartigan-O'Connor, Meneses, Pedersen, Rubenstein, German, Development, 125:2213-2221, 1998.

Swarovsky et al., “Sodium butyrate induces neuroendocrine cytodifferentiation in the insulinoma cell line RINm5F,” Pancreas, 9:460-468, 1994.

Symons, “Avacado sunblotch viroid: primary sequence and proposed secondary structure.” N.A.R., 9:6527-6537, 1981.

Symons, “Small catalytic RNAs,” Annu. Rev. Biochem., 61:641-671, 1992.

Szeto et al., Genomics, 67:221-227, 2000.

Takeda et al., “Organization of the human GLUT-2 (Pancreatic Beta-cell and hepatocyte) glucose transporter gene,” Diabetes, 42:773-777, 1993.

Takeuchi et al., “Expression of human pancreatic peptide in heterologous cell lines,” J.B.C., 266:17409-17415, 1991.

Tang, Sharp, “Atypical protein kinase C isozyme zeta mediates carbachol- stimulated insulin secretion in RINm5F cells,” Diabetes, 47:905-912, 1998.

Temin, “Retrovirus vectors for gene transfer: Efficient integration into and expression of exogenous DNA in vertebrate cell genome,” In: Gene Transfer, Kucherlapati (ed.), New York: Plenum Press, pp. 149-188, 1986.

Thompson, J. D. et al., “Ribozymes in gene therapy.” Nature Medicine, 1:277-278, 1995.

Thorens et al., “Cloning and functional expression in bacteria of a novel glucose transporter present in liver, intestine, kidney and beta-pancreatic islet cells.” Cell, 55:281-290, 1988.

Thorens et al., “Reduced expression of the liver/beta-cell glucose transporter isoform in glucose-insensitive pancreatic beta-cells of diabetic rats,” Proc. Nat'l Acad. Sci. USA, 87:6492-6496, 1990.

Thorens et al., “The loss of GLUT-2 expression by glucose-unresponsive beta cells of db/db mice is reversible and is induced by the diabetic environment,” J. Clin. Invest., 90:77-85, 1992b.

Thorens, “Expression cloning of the pancreatic betacell receptor for the gluco-incretion hormone glucagon-like peptide 1,” P.N.A.S., 89:8641-8646, 1992.

Thorne et al., “Expression of mouse proopiomelanocortin in an insulinoma cell line,” J.B.C., 264:3545-3552, 1989.

Ting and Lee, “Human gene encoding the 78,000-Dalton glucose-regulated protein and its pseudo gene: Structure, conservation, and regulation,” DNA, 7:275-286, 1989.

Top et al., “Immunization with live types 7 and 4 adenovirus vaccines. II. Antibody response and protective effect against acute respiratory disease due to adenovirus type 7,” J. Infect. Dis., 124:155-160, 1971.

Trinh, Minassian, Lange, O'Doherty, Newgard, “Adenovirus-mediated expression of the catalytic subunit of glucose-6-phosphatase in INS-1 cells. Effects on glucose cycling, glucose usage, and insulin secretion,” J. Biol Chem., 272: 24837-24842, 1997.

Tsukuda, Bauchwitz, Holloman, “Isolation of the REC1 gene controlling recombination in Ustilago maydis,” Gene, 85:335-341, 1989.

Tur-Kaspa et al., “Use of electroporation to introduce biologically active foreign genes into primary rat hepatocytes,” Mol. Cell Biol., 6:716-718, 1986.

Unger and Orci, “Glucagon and the Alpha cell,” N.E.J.M., 304:1518-1524, 1981.

Unger, “Daibetic hyperglycemia: Link to impaired glucose transport in pancreatic Beta cells,” Science, 251:1200-1205, 1991.

Usdin et al., “Gastric inhibitory peptide receptor, a member of the secretin-vasoactive intestinal peptide receptor family, is widely distributed in peripheral organs and the brain,” Endocrinology, 133:2861-2870, 1993.

van Wezel, “Growth of cell-strains and primary cells on microcarriers in homogeneous culture,” Nature, 216:64-65, 1967.

Varmus et al., “Retroviruses as mutagens: Insertion and excision of a nontransforming provirus alter the expression of a resident transforming provirus,” Cell, 25:23-36, 1981.

Vollenweider et al., “Processing of proinsulin by furin, PC2 and PC3 in (co)transfected COS (monkey Kidney) cells,” Diabetes, 44:1075-1080, 1995.

Vosberg and Eckstein, “Effect of deoxynucleoside phosphorothioates incorporated in DNA on cleavage by restriction enzymes,” J. Biol. Chem., 257:6595-6599, 1982.

Voss-McGowan, Xu, Epstein, “Insulin synthesis, secretory competence, and glucose utilization are sensitized by transgenic yeast hexokinase,” J. Biol. Chem., 269:15814-15818, 1994.

Waeber et al., “Characterization of the murine high Kₘ glucose transporter GLUT-2 gene and its transcriptional regulation by glucose in a differentiated insulin-secreting cell line,” J. Biological Chemistry, 43:26912-26919, 1994.

Wagner et al., Science, 260:1510-1513, 1990.

Walker et al., “Strand displacement amplification—an isothermal, in vitro DNA amplification technique,” Nucleic Acids Res. 20(7): 1691-1696, 1992.

Wang et al., “Design and performance of a packed bed bioreactor for the production of recombinant protein using serum-free medium,” Proceeding of the Japanese Society for Animal Cell Technology, 1994.

Wang et al., “High density perfusion culture of hybridoma cells for production of monoclonal antibodies in the CelliGen packed bed reactor, In: Animal Cell Technology: Basic & Applied Aspects, S. Kaminogawa et al., (eds), vol. 5, pp. 463-469, Kluwer Academic Publishers, Netherlands, 1993.

Wang et al, “Modified CelliGen-packed bed bioreactor for hybridoma cell cultures,” Cytotechnology, 9:41-49, 1992.

Watada et al., “The human glucokinase gene β-cell-type promoter,” Diabetes, 15:1478-1488, 1996

Welsh et al., “Stimulation of growth hormone synthesis by glucose in islets of Langerhans isolated from transgenic mice,” J.B.C., 261:12915-12917, 1986.

White et al., Arch. Bioch. Biophys. 259:402-411, 1987.

White et al., Arch. Bioch. Biophys. 277:26-34, 1990.

Whitesell et al., “Transport and metabolism of glucose in an insulin-secreting cell line, BetaTC-1,” Biochemistry, 30:11560-11566, 1991.

Willnow and Herz, “Homologous Recombination for Gene replacement in Mouse Cell Lines,” Methods in Cell Biology, 43 pt A:305-334, 1994.

Wilson and Chung, “Rat brain hexokinase: further studies on the specificity of the hexose and hexose-6-phosphate binding sites,” Arch. Biochem. Biophys., 269:517-525, 1989

Wilson, “Brain hexokinase: A proposed relation between soluble-particulate distribution and activity in vivo,” J. Biol. Chem., 243:3640-3647, 1968.

Wilson, “Hexokinases,” In: Reviews of Physiology, Biochemistry and Pharmacology, Pette, D. (Ed.), 126:65-174, 1994.

Wilson, “Ligand induced confirmations of rat brain hexokinase: Effects of glucose-6-phosphate and inorganic phosphate,” Arch. Biochem. Biophys., 159:543-549, 1973.

Wilson, “Regulation of mammalian hexokinase activity,” In: Regulation of Carbohydrate Metabolism, Beitner, R. (Ed.) Vol. 1, CRC, Boca Raton, 45-81, 1985.

WO 88/10315

WO 89/01967

WO 89/01967

WO 89/06700

WO 90/02580

WO 90/02580

WO 90/07641

WO 90/15637

WO 90/15637

WO 91/10425

WO 91/10425

WO 91/10470

WO 91/10470

Wong et al., “Appearance of β-lactamase activity in animal cells upon liposome mediated gene transfer,” Gene, 10:87-94, 1980.

Wu and Wu, “Evidence for targeted gene delivery to HepG2 hepatoma cells in vitro,” Biochemistry, 27:887-892, 1988.

Wu and Wu, “Receptor-mediated in vitro gene transfections by a soluble DNA carrier system,” J. Biol. Chem., 262:4429-4432, 1987.

Wu and Wu, Adv. Drug Delivery Rev., 12:159-167, 1993.

Xie and Wilson, “Rat brain hexokinase: the hydrophobic N-terminus of the mitochondrially bound enzyme is inserted in the lipid bilayer,” Arch. Biochem. Biophys., 267:803-810, 1988.

Yajima et al., “cAMP enhances insulin secretion by an action on the ATP-sensitive K⁺ channel-independent pathway of glucose signaling in rat pancreatic islets,” Diabetes, 48:1006-1012, 1999.

Yang et al., “Cellular immunity to viral antigens limits E1-deleted adenoviruses for gene therapy,” P.N.A.S. USA, 91:4407-4411, 1994.

Yang et al, “In vivo and in vitro gene transfer to mammalian somatic cells by particle bombardment,” Proc. Nat'l Acad. Sci. USA, 87:9568-9572, 1990.

Yoon, Cole-Strauss, Kmiec, “Targeted gene correction of episomal DNA in mammalian cells mediated by a chimeric RNA-DNA oligonucleotide,” Proc. Nat'l Acad. Sci. USA, 93:2071-1076, 1996.

Yuan and Altman, “Selection of guide sequences that direct efficient cleavage of mRNA by human ribonuclease P,” Science, 263:1269-1273, 1994.

Yuan et al., “Targeted cleavage of mRNA by human RNase P.” P.N.A.S., 89:8006-8010, 1992.

Yun and Eipper, “Addition of an endoplasmic reticulum retention/retrieval signal does not block maturation of enzymatically active peptidylglycine alpha-amidating monooxygenase,” J.B.C., 270:15412-15416, 1995.

Zawalich and Zawalich, “Regulation of insulin secretion by phospholipase C.” Am J Physiol., 271:E409-E416, 1996.

Zelenin et al, “High-velocity mechanical DNA transfer of the chloramphenicol acetyltransferase gene into rodent liver, kidney and mammary gland cells in organ explants and in vivo,” FEBS Lett., 280:94-96, 1991.

Zhou et al. , “In vitro and in vivo evaluation of insulin-producing βTC6-F7 cells in microcapsules.: Am J. Physiol. 1998 43: C1356-C1362.

What is claimed is:

1. An isolated and purified polynucleotide encoding a polypeptide having an amino acid sequence encoded by SEQ ID NOS:1-7, 66, 67, 69, 70, 72 or 74.

2. The polynucleotide of claim 1, wherein the polynucleotide is selected from SEQ ID NOS:1-7, 66, 67, 69, 70, 72 or 74, or the complement thereof.

3. An expression cassette comprising (a) a polynucleotide encoding a polypeptide having an amino acid sequence encoded by SEQ ID NOS:1-7, 66, 67, 69, 70, 72 and 74 and (b) a promoter, wherein said polynucleotide is positioned so as to be under the regulatory control of said promoter.

4. The expression cassette of claim 3, wherein the polynucleotide is selected from SEQ ID NOS:1-7, 66, 67, 69, 70, 72 or 74.

5. The expression cassette of claim 3, wherein said promoter is CMV, SV40 IE, RSV, GAPHD, or RIP1.

6. The expression cassette of claim 3, further comprising a polyadenylation signal.

7. The expression cassette of claim 3, further comprising a heterologous leader sequence.

8. The expression cassette of claim 3, wherein said expression cassette is further comprised within an expression vector comprising an origin of replication.

9. The expression cassette of claim 8, wherein said origin of replication is a bacterial origin or replication or a viral origin of replication.

10. The expression cassette of claim 8, wherein said expression vector is a viral vector.

11. The expression cassette of claim 10, wherein said viral vector is selected from the group consisting of an adenoviral vector, a retroviral vector, a vaccinia viral vector, a herpesviral vector, an adeno-associated viral vector, and a polyoma viral vector.

12. The expression cassette of claim 8, wherein said expression vector is a prokaryotic plasmid vector.

13. The expression cassette of claim 8, further comprising a positive-selectable marker gene.

14. The expression cassette of claim 8, further comprising a negative-selectable marker gene.

15. The expression cassette of claim 8, further comprising an internal ribosome entry site.

16. An oligonucleotide of 15 to about 50 bases, comprising 15 or more consecutive bases of SEQ ID NOS:1-7, 66, 67, 69, 70, 72 or 74, or the complement thereof.

17. The oligonucleotide of claim 16, wherein said oligonucleotide is 15, 16, 17, 18, 19, 20, 21, 22, 23, 24, 25, 30, 35, 40, 45 or 50 bases in length.

18. The oligonucleotide of claim 16, wherein said oligonucleotide comprises 15, 16, 17, 18, 19, 20, 21, 22, 23, 24, 25, 30, 35, 40, 45 or 50 consecutive bases of SEQ ID NOS:1-7, 66, 67, 69, 70, 72 or 74, or the complement thereof.

19. The oligonucleotide of claim 16, further comprising a restriction enzyme site.

20. The oligonucleotide of claim 16, wherein the entire oligonucleotide base pairs with SEQ ID NOS:1-7, 66, 67, 69, 70, 72 or 74, or the complement thereof.

21. A cell comprising a heterologous polynucleotide encoding a polypeptide having an amino acid sequence encoded by SEQ ID NOS:1-7, 66, 67, 69, 70, 72 or 74.

22. The cell of claim 21, wherein said cell is a eukaryotic cell.

23. The cell of claim 21, wherein said cell is a prokaryotic cell.

24. The cell of claim 21, wherein said polynucleotide further comprises a promoter active in said cell, said promoter positioned so as to provide regulatory control for the region encoding said polypeptide.

25. The cell of claim 24, further comprising a positive- and/or negative-selectable marker gene.

26. A method of expressing a polypeptide comprising:
(a) providing a host cell, said host cell comprising a heterologous an expression cassette comprising (a) a polynucleotide encoding a polypeptide having an amino acid sequence encoded by SEQ ID NOS:1-7, 66, 67, 69, 70, 72 or 74 and (b) a promoter active in said host cell, wherein said polynucleotide is positioned so as to be under the regulatory control of said promoter; and
(b) culturing said host cell under conditions supporting the expression of said polypeptide.

27. The method of claim 26, wherein said host cell is a prokaryotic host cell.

28. The method of claim 26, wherein said host cell is a eukaryotic host cell.

29. The method of claim 26, further comprising isolating said polypeptide from said host cell.

30. The method of claim 26, wherein said promoter is an inducible promoter, and said conditions further comprise induction of said promoter.

31. A polypeptide having an amino acid sequence encoded by SEQ ID NOS:1-7, 66, 67, 69, 70, 72 or 74.

32. The polypeptide of claim 31, fused to a heterologous peptide or polypeptide sequence.

33. A peptide of 5 to about 35 residues comprising 5 or more consecutive residues of a polypeptide encoded by SEQ ID NOS:1-7, 66, 67, 69, 70, 72 or 74.

34. The peptide of claim 33, wherein said peptide is 5, 6, 7, 8, 9, 10, 11, 12, 13, 14, 15, 16, 17, 18, 19, 20, 21, 22 23, 24, 25, 26, 27, 28, 29, 30, 31, 32, 33 34 or 35 residues in length.

35. The peptide of claim 33, wherein said peptide comprises 5, 6, 7, 8, 9, 10, 11, 12, 13, 14, 15, 16, 17, 18, 19, 20, 21, 22 23, 24, 25, 26, 27, 28, 29, 30, 31, 32, 33 34 or 35 consecutive residues of a polypeptide encoded by SEQ ID NOS:1-7, 66, 67, 69, 70, 72 or 74.

36. The peptide of claim 33, linked to a carrier molecule.

37. A monoclonal antibody that binds immunologically to a polypeptide encoded by SEQ ID NOS:1-7, 66, 67, 69, 70, 72 or 74.

38. The monoclonal antibody of claim 37, wherein said monoclonal antibody is a single-chain monoclonal antibody.

39. A nucleic acid encoding single chain monoclonal antibody that binds immunologically to a polypeptide encoded by SEQ ID NOS:1-7, 66, 67, 69, 70, 72 or 74.

40. A polyclonal antibody composition, antibodies of which bind immunologically to a polypeptide having an amino acid sequence encoded by SEQ ID NOS:1-7, 66, 67, 69, 70, 72 or 74.

41. A method for modulating the glucose-responsiveness of a cell comprising introducing into said cell an expression cassette comprising (a) a polynucleotide encoding a polypeptide having an amino acid sequence encoded by SEQ ID NOS:1-7, 66, 67, 69, 70, 72 or 74 and (b) a promoter active in said cell, wherein said polynucleotide is positioned so as to be under the regulatory control of said promoter.

42. The method of claim 41, wherein said cell is an insulin-producing cell.

43. The method of claim 41, wherein said cell is a rat cell.

44. The method of claim 41, wherein said cell is a human cell.

45. The method of claim 41, wherein said cell further comprises a positive- and/or negative-selectable marker gene.

46. The method of claim 41, wherein said cell is immortalized.

47. The method of claim 41, wherein said cell further comprises one or more heterologous coding regions for insulin, GLUT-2, glucokinase, insulin-like growth factor GLP-1, IPF1, PC2, PC3, PAM, glucagon-like peptide I receptor, glucose-dependent insulinotropic polypeptide receptor, KIR, SUR, GHRFR, glucagon receptor, NKX2.2, NKX6.1 or GHRHR.

48. The method of claim 41, wherein said promoter is an inducible and said method further comprises induction.

49. The method of claim 41, wherein said cell further comprises a positive- and/or negative-selectable marker gene.

50. The method of claim 41, further comprising introducing said cell into a mammalian host organism.

51. The method of claim 50, wherein said cell is encapsulated in a biocompatible device.

52. The method of claim 50, wherein said biocompatible device comprises a permeable membrane.

53. A method of screening for a modulator of expression of a polypeptide encoded by SEQ ID NOS:1-7, 66, 67, 69, 70, 72 or 74 comprising:
(a) providing a cell that expresses a polypeptide encoded by SEQ ID NOS:1-7, 66, 67, 69, 70, 72 or 74;
(b) treating said cell with a candidate modulator;
(c) determining the effect on expression of a polypeptide encoded by SEQ ID NOS:1-7, 66, 67, 69, 70, 72 or 74, as compared to a similar cell not treated with said candidate modulator,
wherein a difference between the expression in treated and untreated cells indicates that said candidate modulator is a modulator of expression of a polypeptide encoded by SEQ ID NOS:1-7, 66, 67, 69, 70, 72 or 74.

54. A method for measuring the expression of a polypeptide encoded by SEQ ID NOS:1-7, 66, 67, 69, 70, 72 or 74 in a cell comprising:
(a) obtaining an mRNA population from said cell;
(b) contacting said population with a DNA probe that hybridizes to SEQ ID NOS:1-7, or the complement thereof; and
(c) measuring hybridization of said probe to members of said population,
whereby measuring hybridization of said probe measures the expression of a polypeptide encoded by SEQ ID NOS:1-7, 66, 67, 69, 70, 72 or 74.

55. The method of claim 54, further comprising amplification of members of said population.


SEQUENCE LISTING (75 sequences)

SEQ ID NO: 1 - DNA, 1105 bp, RAT
  modified_base (1096): N = A, C, G, OR T/U
gttcagtgtctgagatctcctctgctagtgtgtaacaaggttagatttagagtttacacctgaccatacctgggccgtgtgcctcaggattctgccattgaacagcaggaagggagggatgaggttttcaggagaggagtcacctgtcatcccataatatggcactcaagggatgagctgacactgttgatgcctgcagggagttttgcagtggagcagggactaaggagtttttgtgttctttcttcctgagatgggactgtcagaggtagcatgaacccaacgcctttaaggaatgctgagggccatcggcgggaggagcggtagggcttggctttatttccacctttctgctgctgcaggaggcacgtttttgagactcacttctccagaagagcatgggagcagaacccacaccctcagctatcaatatgtacatgaatataaaaatatgaccagggtccacaccttccctttgcaggatattgccaaacattccaggttttatcacactttcatggagttccagaaacctctgggctggtccatggacttccgtgcaaaatcttttattagcaagaacctactacccccacacctgacatatctgatcaccttccttatcctacccagtgacgtctcaactccctgccctgtgtccagcaagactgctgggtcagagtggagagaattcagctcatccttatgtttccatgggaacctttccttcccatggacctgatcctgattcttgccgatagacaacagcttgcccacactgtgtttggaattagactgtatcttcaccccttaatcttcataggaaaacccaaggctggtgacatggctcagctgggtaagggtgatggtggccaagcatgatgacttgaatttggtccctgatactgggcattctttccgtcacgggaaggagagggctgactcctgcaagttgtcttttgatctccacaagtgcacatcccccttcctgcaaaataaataacctaatgtaatttaagaagatgaagacgaaaaaacactgtcaaggtccttatatctatcgagacagtcctcaataaaatttttcttacacagncaaaaaaaa

SEQ ID NO: 2 - DNA, 397 bp, RAT
  modified_base (17): N = A, C, G, OR T/U
tttgcttttattaaaanctgttttaaagacagctggtgttcacacctctgttgtcagcaaaacaactagttaggcacagacacatagttccaagtagttaaaatcacctgattacaaacactcttccttatcgtctctgtagatcctctatgcaggacggtacaaatttgcgggacttgctctggtaaaacacagatatgggttatgtctgacatccaaattataactgatatttatgggtaacaacagctaaggtccttagaatgaagaatgtattgtattgagggatagaaactgatcatacaatgggtaacaaccacagagctcgaagatttgtgattgttaaaacttctctggcatttcatcattaataaacatctgtgttgtgacagcagca

SEQ ID NO: 3 - DNA, 1588 bp, RAT
  modified_base (201)..(1581): N = A, C, G, OR T/U
gctaatgaagagccccaaggaaagaaagacctacccatcgctctgaatcctcttccccaccatgccacagacctgagggcacgtgacccgggagtgtgcatcagaagataatactaccttcagagaactctacttataggcaagtagaggaactggacagacaagaacagtcctgccagttgatcaatgacttggggtgtnttcaacgagatgcttcgtttaaagtgcttaccacaagccagttattgttctaggtgcttcagaacgaagacctggtcctattcatcggtatcctggcaggaaacagatggcgtacacaaattggatacactgaagtgtaccgactgaaaaggcaaagcgctgcatggctctggcgaatccagtaagggatggtgcaacaccccgcctcaccgcagcacggcgcctgtcaccctacactgcctgcaggaactcataaggggatcctcacacacacacccggatgtaccccagggagagcttcttcccagaggcatcttggcaagagccttgggttgcagacaattcctggccacctggtagaaagcgaagggtgaataaacacctccaacctcactcttcccctgccttgggctctctggctgccgtctcctctcagctaaccaacaaaagaccagaaggcaaagcgccccttgatgccggatgtatggggttggtcttcgaaagcatagaacggggaagagaactgttgaaatctgggccaaagggatcctatgggtactctcacatcacccctaccttaacggtccttaattacatgcattctacagaacggacacacagggaggttacgaacttcttccaaaacagaacagacaagaaaaaacaacaacaacaacaacaacaaaaacttgttcaaggtcataggtgctcacacttgaacacgtaaatgttcaaggtcacaagagctcataggtgacagacttaggtgttcctcccaggtattttattctttcatatttcacactatgcacttacatatcttactatagaaytctctttttaaacagaggcaaaacacatttctttgggagctgaagagatggctcagcagttagagcgcttgctgttcttgtaaaggacctgagttcatccccagaacccacaagtatcaccctctcctggttgccacggacatctgtcaaagagcattttactaatggatcaattccgtggtagacctactccccggaaccttcttgacagagtttggtagattgcagccacaggraactcagaggaaggaactttggtcgagtttgcgagggcccagttacaaatgtggacgacgaattcattaagttttccacacacgacttattttagattccaaaataatcgtgggaccctggcccttcttaataaagggatgagctcccgttgtagatttggggtgggtattcgagctatttaaaaaaaagtcttgagtttggtataaaacaaatgcaccccagcaaataactgnaaagcaggntctaaaagaaagccacacttggtatcttgtcacggggttgaaatggntttttttggggnaaaaaatanaaaaaaa

SEQ ID NO: 4 - DNA, 1169 bp, RAT
  modified_base (4)..(90): N = A, C, G, T or U
aatnnnnntnnctnnncnnntgatcgacatcargtgtcgggcgggcacattgaaaggttatgtgaaatgaaaagacatgatchccctgbnttgtggcatgggccagatcccgtgttggcatgggcaagagtgtctttttgtgtgtttcctcaggaccagcaagatccgttgtgggtccctgaaatattgaccaggaagtacaagaagaatgaagatcctgctggtgctcacacttctcggtgtgacccaaatgcttggtccaaatggaacccctgtgggggatattatcggtgttcccgagactcatgccgatacatcatgacttttaagtccactcctctactttgtctaaacagttgtcaatttatcttctaggtaattggacaggagaattcgatttcttaatggatcagctacgtgtatccatcatgaccgtgaactccacacgtgcagatgctagactggctatgggattgtcatcttggattgaggctgctatgaatcatctgagggagtgggcgggtatgggaaccctagctagactcttgctgctggtctccctagtgcgcctgtggtgtctctgttgaatgaggtttgttcagaggcggcacgcagttatggtggtacaggtatttgcagccattaaatcaggacagtcccctcaagcctggcttacggtcattcagaagaccttgttgkacctcacgcctgagtatgtatkragtgaccctgcccagtgatgggsmamcgcccttytacctaaggcatgggaccttgtggagacgaggtgtcctaaractgctttaatgaacaaatgatcctaagaacggttggatgggtagccacgctcaggggtgctttgcctgcttgattaattaaacaaaaaaggggaactataggcagttgcgacatggtgcacctcaaagatggtgctggtttccgccttccaccctcctaatggtgcgtggtccttgtagcaaggaagtccttatttggctatgcctgcctggcctcccagcttccgcatagtgagagccgatcttcatgacacacgtagcttgctgggattggccagcagtagctatttaaatgtgctccagggtttccaagggggcagaagattgtatcaaggttcctgaataaaactgcttgaagaagaaaaaaaaaaa

SEQ ID NO: 5 - DNA, 1197 bp, RAT
  modified_base (1)..(88): N = A, C, G, T or U
nccntgaacaggagggtcagccganntnngttaaccatgggtnagcaatagntcgccngntggacaggtnttgcactcctagaacctnggtttattgcacaggattagagtgtcgggcttgagcagcccatgagggatgacgagcttggcatcgcacagggaagctttaccaaacacgctccctggaaggcatgtcatattacatgagggtatctgccccagttttccctccctcgaaaaaatggcagtgcgacgggtcgggagtgccctgggtcacaacaacagcttaagggtatctctcttgtacaggtttgccaactttgtgcgaggatatgacctctgccttcaccctcctgtttatgggtgtcctatttgcttaaaaacagataagggggagatgtggagggccgtcctcacattctccactgtaagatggcaccgatatccggcaggatgcacctaataaaagggcaatacgcaagtgcctggtaaattcctcactcagggggacacgtatgctgtggtacgtcatctggcacatctggcagcgaccagccagagagtgacacgtcctaggtgaggatagcttcctatataagggatggtctttccctcactcgaggtctccgtattgtaagcttgtgctctcgctctcaagatgcattaaagctttttctgcagaaggatcctttgtgtcctgcgttattcttgctggcgagacgtagtgtgggacaatggttcctgtatgggtctgagctaagtcttctgcaaatactttatggttgtatagcttggtgttctgtggaactccccacccaaatgggccaggggttgtcgctgattcttttgcctgtgcttgagatcatttttctcctatgtgtcgccacatctagttgtgataagggaggttttgcccagttttactgcatcttcttaggccatattagataactgtttctaggagaattgttcttttttttctttctctatcttttgaaaagaaacagagggtacttggtctaggagaaaccaagggaagggactgtaaaagtggagagaggggaaactatggtcatgatgtaatgaataaaagaaaaattaaagtttaaaaaaatgaaccaataaggtacttgctaagtagataaaactcttcattttgtttaaaaataagtgtaataaattatgaatgttacaaaa

SEQ ID NO: 6 - DNA, 514 bp, RAT
  modified_base (423)..(510): N = A, C, G, OR T/U
gatcctggccagagatgagaggactctggctccatgtttgtgtcatgctacacagagggaagcaaaacactgagaacctgagaccgtgaatagtgagaaggaggtctctgctgccacctccaccctcctttctgagcaagctcccaagatacctgtcctgaggtgatgccacataagtctctggtacccacagtcttcttgttccagcaaccctttctgtgtctgactctctcagggtctcatgtattccaggttggtctcaaagcccatagcctttgaagataacctttgagtctctgatcctcctgcttcgactctctgagtgttcacacagtgctgggcacaaaacccaaagatttgtgcccaccagggagacactctaccagtgaagctacgtctcctgccgcagatttattatatttnaaaaactttccatgacagtaaaatttataatcatataaaaaaaaaaaaaaaaatttatttttttttgttttnnaaaaaaaaaaaannaaaa

SEQ ID NO: 7 - DNA, 329 bp, RAT
gatctggtccctctctgtcctaacaggctctgagccatcgctgaaagagagtcctctgaggattgagggaagtccatagcccgtactctgacttccaggaaggtttgaggaaggcggtcctcttcttgtggtgcctctagtaggagcagcccacacttgggcccagctcccactatcaggaaaagacaggtggctctggtctttgcacagctccagcggaatgaacagtgcatctctcctctctctcttgcaggaagtctcagcctctggtgcaggtgatgttcccaaggcagaggaaagggaagtgcagagtgaggactcccacgatc

SEQ ID NO: 8 - DNA, 21 bp, Artificial Sequence
  Description of Artificial Sequence  Synthetic Primer
cactgtcaaggtccttatatc

SEQ ID NO: 9 - DNA, 19 bp, Artificial Sequence
  Description of Artificial Sequence  Synthetic Primer
gcattctttccgtcacggg

SEQ ID NO: 10 - DNA, 21 bp, Artificial Sequence
  Description of Artificial Sequence  Synthetic Primer
gacttgaatttggtccctgat

SEQ ID NO: 11 - DNA, 19 bp, Artificial Sequence
  Description of Artificial Sequence  Synthetic Primer
gtgtccagcaagactgctg

SEQ ID NO: 12 - DNA, 21 bp, Artificial Sequence
  Description of Artificial Sequence  Synthetic Primer
cactgtcaaggtccttatatc

SEQ ID NO: 13 - DNA, 21 bp, Artificial Sequence
  Description of Artificial Sequence  Synthetic Primer
attcttgccgatagacaacag

SEQ ID NO: 14 - DNA, 21 bp, Artificial Sequence
  Description of Artificial Sequence  Synthetic Primer
gaattagactgtatcttcacc

SEQ ID NO: 15 - DNA, 20 bp, Artificial Sequence
  Description of Artificial Sequence  Synthetic Primer
aatttggtccctgatactgg

SEQ ID NO: 16 - DNA, 19 bp, Artificial Sequence
  Description of Artificial Sequence  Synthetic Primer
gtgtccagcaagactgctg

SEQ ID NO: 17 - DNA, 21 bp, Artificial Sequence
  Description of Artificial Sequence  Synthetic Primer
cactgtcaaggtccttatatc

SEQ ID NO: 18 - DNA, 22 bp, Artificial Sequence
  Description of Artificial Sequence  Synthetic Primer
gtattgagggatagaaactgat

SEQ ID NO: 19 - DNA, 20 bp, Artificial Sequence
  Description of Artificial Sequence  Synthetic Primer
ggtaacaacagctaaggtcc

SEQ ID NO: 20 - DNA, 20 bp, Artificial Sequence
  Description of Artificial Sequence  Synthetic Primer
gacttgctctggtaaaacac

SEQ ID NO: 21 - DNA, 20 bp, Artificial Sequence
  Description of Artificial Sequence  Synthetic Primer
catctgtgttgtgacagcag

SEQ ID NO: 22 - DNA, 19 bp, Artificial Sequence
  Description of Artificial Sequence  Synthetic Primer
cctctatgcaggacggtac

SEQ ID NO: 23 - DNA, 20 bp, Artificial Sequence
  Description of Artificial Sequence  Synthetic Primer
ggtaacaacagctaaggtcc

SEQ ID NO: 24 - DNA, 20 bp, Artificial Sequence
  Description of Artificial Sequence  Synthetic Primer
gacttgctctggtaaaacac

SEQ ID NO: 25 - DNA, 20 bp, Artificial Sequence
  Description of Artificial Sequence  Synthetic Primer
catctgtgttgtgacagcag

SEQ ID NO: 26 - DNA, 20 bp, Artificial Sequence
  Description of Artificial Sequence  Synthetic Primer
ggtgcttcagaacgaagacc

SEQ ID NO: 27 - DNA, 21 bp, Artificial Sequence
  Description of Artificial Sequence  Synthetic Primer
agccagttattgttctaggtg

SEQ ID NO: 28 - DNA, 20 bp, Artificial Sequence
  Description of Artificial Sequence  Synthetic Primer
gcaagtagaggaactggaca

SEQ ID NO: 29 - DNA, 20 bp, Artificial Sequence
  Description of Artificial Sequence  Synthetic Primer
tctacagaacggacacacag

SEQ ID NO: 30 - DNA, 19 bp, Artificial Sequence
  Description of Artificial Sequence  Synthetic Primer
ggtagattgcagccacagg

SEQ ID NO: 31 - DNA, 20 bp, Artificial Sequence
  Description of Artificial Sequence  Synthetic Primer
cacttggtatcttgtcacgg

SEQ ID NO: 32 - DNA, 19 bp, Artificial Sequence
  Description of Artificial Sequence  Synthetic Primer
cactgcctgcaggaactca

SEQ ID NO: 33 - DNA, 20 bp, Artificial Sequence
  Description of Artificial Sequence  Synthetic Primer
caggaactcataaggggatc

SEQ ID NO: 34 - DNA, 20 bp, Artificial Sequence
  Description of Artificial Sequence  Synthetic Primer
cgaatccagtaagggatggt

SEQ ID NO: 35 - DNA, 19 bp, Artificial Sequence
  Description of Artificial Sequence  Synthetic Primer
catcggtatcctggcagga

SEQ ID NO: 36 - DNA, 20 bp, Artificial Sequence
  Description of Artificial Sequence  Synthetic Primer
gcaagtagaggaactggaca

SEQ ID NO: 37 - DNA, 20 bp, Artificial Sequence
  Description of Artificial Sequence  Synthetic Primer
tctacagaacggacacacag

SEQ ID NO: 38 - DNA, 20 bp, Artificial Sequence
  Description of Artificial Sequence  Synthetic Primer
cataggtgctcacacttgaa

SEQ ID NO: 39 - DNA, 21 bp, Artificial Sequence
  Description of Artificial Sequence  Synthetic Primer
ctataggcagttgcgacatgg

SEQ ID NO: 40 - DNA, 18 bp, Artificial Sequence
  Description of Artificial Sequence  Synthetic Primer
tgcacctcaaagatggtg

SEQ ID NO: 41 - DNA, 19 bp, Artificial Sequence
  Description of Artificial Sequence  Synthetic Primer
gttgtacctcacgcctgag

SEQ ID NO: 42 - DNA, 20 bp, Artificial Sequence
  Description of Artificial Sequence  Synthetic Primer
ttgatcgacatcaagtgtcg

SEQ ID NO: 43 - DNA, 18 bp, Artificial Sequence
  Description of Artificial Sequence  Synthetic Primer
gcttccgcatagtgagag

SEQ ID NO: 44 - DNA, 20 bp, Artificial Sequence
  Description of Artificial Sequence  Synthetic Primer
ggtccttgtagcaaggaagt

SEQ ID NO: 45 - DNA, 18 bp, Artificial Sequence
  Description of Artificial Sequence  Synthetic Primer
tgcacctcaaagatggtg

SEQ ID NO: 46 - DNA, 19 bp, Artificial Sequence
  Description of Artificial Sequence  Synthetic Primer
gttgtacctcacgcctgag

SEQ ID NO: 47 - DNA, 20 bp, Artificial Sequence
  Description of Artificial Sequence  Synthetic Primer
cttgatcgacatcaagtgtc

SEQ ID NO: 48 - DNA, 20 bp, Artificial Sequence
  Description of Artificial Sequence  Synthetic Primer
ctgattcttttgcctgtgct

SEQ ID NO: 49 - DNA, 20 bp, Artificial Sequence
  Description of Artificial Sequence  Synthetic Primer
gcttggtgttctgtggaact

SEQ ID NO: 50 - DNA, 20 bp, Artificial Sequence
  Description of Artificial Sequence  Synthetic Primer
gtctgagctaagtcttctgc

SEQ ID NO: 51 - DNA, 22 bp, Artificial Sequence
  Description of Artificial Sequence  Synthetic Primer
ccaataaggtacttgctaagta

SEQ ID NO: 52 - DNA, 19 bp, Artificial Sequence
  Description of Artificial Sequence  Synthetic Primer
gcaatacgcaagtgcctgg

SEQ ID NO: 53 - DNA, 20 bp, Artificial Sequence
  Description of Artificial Sequence  Synthetic Primer
gtgtcctgcgttattcttgc

SEQ ID NO: 54 - DNA, 20 bp, Artificial Sequence
  Description of Artificial Sequence  Synthetic Primer
gtctgagctaagtcttctgc

SEQ ID NO: 55 - DNA, 20 bp, Artificial Sequence
  Description of Artificial Sequence  Synthetic Primer
agcttggtgttctgtggaac

SEQ ID NO: 56 - DNA, 22 bp, Artificial Sequence
  Description of Artificial Sequence  Synthetic Primer
caataaggtacttgctaagtag

SEQ ID NO: 57 - DNA, 19 bp, Artificial Sequence
  Description of Artificial Sequence  Synthetic Primer
gcaatacgcaagtgcctgg

SEQ ID NO: 58 - DNA, 22 bp, Artificial Sequence
  Description of Artificial Sequence  Synthetic Primer
gatcagagactcaaaggttatc

SEQ ID NO: 59 - DNA, 20 bp, Artificial Sequence
  Description of Artificial Sequence  Synthetic Primer
caacctggaatacatgagac

SEQ ID NO: 60 - DNA, 20 bp, Artificial Sequence
  Description of Artificial Sequence  Synthetic Primer
gctggaacaagaagactgtg

SEQ ID NO: 61 - DNA, 19 bp, Artificial Sequence
  Description of Artificial Sequence  Synthetic Primer
cagagtcctctcatctctg

SEQ ID NO: 62 - DNA, 21 bp, Artificial Sequence
  Description of Artificial Sequence  Synthetic Primer
ctctgtgtagcatgacacaaa

SEQ ID NO: 63 - DNA, 20 bp, Artificial Sequence
  Description of Artificial Sequence  Synthetic Primer
cacctcaggacaggtatctt

SEQ ID NO: 64 - DNA, 19 bp, Artificial Sequence
  Description of Artificial Sequence  Synthetic Primer
gatctggtccctctctgtc

SEQ ID NO: 65 - DNA, 19 bp, Artificial Sequence
  Description of Artificial Sequence  Synthetic Primer
ggaagtgcagagtgaggac

SEQ ID NO: 66 - DNA, 835 bp, RAT
  modified_base (343)..(823): N = A, C, G, T or U
aatctaggggcagaagagacccaagtgtttatcaatacatgaacaaataaacgaaatgtggcatgccatgactgaatagtgggagactattcagtcacaaaaagggagaaaattctgatacatgctctatgtgtaaaagtctaggacattaagctaggtaggaaaggagcagacaccaaaatgacaaatgctatgtggttccagttgtttttggcagtcaaatctgtggatgtagaaataaggatggtagttccaggtgttcctctagcagctgccacctcttcctcttcctcctacctcttacctcttacctcctcctcttcctcttacctcttacctcttncctcctcctcctcatgcttncctcctcctcctcttcattttcctcctcctcctcctcttcttcctcctcctcttcttcctctctttcctcttcctcctcctcttctgcttcttttcttttaactcatttatttattattgtgtgtgtgtgtgtgtgtgtgtgtgtgtgtgtgtgtgtgtggctacatgtgtgtcttggcccacgtatagaaatgagaggacaactttcaggagntgcttcttcccttctaccatgtacgtcccagggattgaacttaggncctcaggcttggtagcaagctcctgtaccatctgacccatctcactggccctgcagccatcctcacccattggcganactgcatggttgtctcagantgcaantttgtaaggattaatggcgngagangccttatgcatggttctgggnggccattcnatanctntggagaaacagaaattgaancncttttccgnnttngagaaacaaaac

SEQ ID NO: 67 - DNA, 1640 bp, RAT
  modified_base (4)..(1628): N = A, C, G, T or U
tgcntttgccagtntgncaatcatnccatcctntggtcatanaagttttaagattgncctgattctgttttgtntatatngtctgttttcccncgtcttctatatgttttcattggcacgtgtgtgagcatggtatcacctaactccattcattttctttttaattttattttttttgcagtactgaggattgaacccagggccttgtgcatgctaggtgagtgctctaccaactgtgctccagcctcagatccctattcactttttaaatttccttgccttgattgccagtatctggttccttactctatcacttccgggtaaagagcctgactcttctatggtgatgtgtgggcactgggatagggtctcaccaagtcacccatgctggcctggaactcacagtcctcttaactcagtccccagagtgctgggactacatgaccacccacccccaggaggcaggggcatgactctgtgtcttccctgatccctgcgtataagccgtgcctgaccacaggcctgcagagcatttgttcagtgtctgagatctcctctgctagtgtgtaacaaggttagatttagagtttacacctgaccatacctgggccgtgtgcctcaggattctgccattgaacagcaggaagggagggatgaggttttcaggagaggagtcacctgtcatcccataatatggcactcaagggatgagctgacactgttgatgcctgcagggagttttgcagtggagcagggactaaggagtttttgtgttctttcttcctgagatgggactgtcagaggtagcatgaacccaacgcctttaaggaatgctgagggccatcggcgggaggagcggtagggcttggctttatttccacctttctgctgctgcaggaggcacgtttttgagactcacttctccagaagagcatgggagcagaacccacaccctcagctatcaatatgtacatgaatataaaaatatgaccagggtccacaccttccctttgcaggatattgccaaacattccaggttttatcacactttcatggagttccagaaacctctgggctggtccatggacttccgtgcaaaatcttttattagcaagaacctactacccccacacctgacatatctgatcaccttccttatcctacccagtgacgtctcaactccctgccctgtgtccagcaagactgctgggtcagagtggagagaattcagctcatccttatgtttccatgggaacctttccttcccatggacctgatcctgattcttgccgatagacaacagcttgcccacactgtgtttggaattagactgtatcttcaccccttaatcttcataggaaaacccaaggctggtgacatggctcagctgggtaagggtgatggtggccaagcatgatgacttgaatttggtccctgatactgggcattctttccgtcacgggaaggagagggctgactcctgcaagttgtcttttgatctccacaagtgcacatcccccttcctgcaaaataaataacctaatgtaatttaagaagatgaagacgaaaaaacactgtcaaggtccttatatctatcgagacagtcctcaataaaatttttcttacacagncaaaaaaaa

SEQ ID NO: 68 - DNA, 3541 bp, Artificial Sequence
  modified_base (1555): N = A, C, G, T or U
gggatgcagcccccccaaaagaaatcggggtgaatcagaaatacctactaatgatgccccaatgcaaaacctgagaagagagagagggaaaaacgacaagcatatatttgtctaaaatcgggaaaaaagaaaaaccatgaaaaatcgagacagaaacacatacgttgaggtctaaccacgaacgataaaggtggaagaaaggacttttctgcctgcaacacccaagcactaagctaatttccttaataaacgaagagctattggaaaccaacaagggaaggacaaaagcaaagcaaaaaagggggcaaaagagaccgcaagtcacaggacaatgcaaatggctggaagaatcttgacctgactgtctgcatagcgaaacgccaacctgagataccgggtgaaggcggtttatggcagcgcgctgaatcgcatgtggtcaaccatcattgcgtgaatccactgcatagaatagcaagaaattgcgaggagagcaagcaggtagattaccaagcctgcctctgatagaagacattcctatgtcaccaggaaaaataacggaggtatcgctgccataaaggacctggatcggaatagatcccaatgaaataaaacccgttgccgaagtttgtagaaacgaaatggttcaccctactccccatttcaacacctttaaaagatattttaaagtattttgagtatctgcagagccagtttgttgttccaacgtacagtgccattttcctgaggctatatgacctgtgataccataacaggctgaaggctcaagcaaatgaaaattccagctgttccctatcaagtctgacaccgaagacatttgccgagatgtaaaacgatgctaccaggtagccacatctgctgtgtgtccatgattgcatgccatgtccagaagacggtgtttcatgtcgcaccttcccatcctgccatcagccctacgtcctacacagtaactcagctgttccctgtacaggtctcccccatgccccttcatctccatgtctccatggctgtacctgtgttcctggtcaccctgtgtctccctcactccccgacttgagtggcctctgatgaactcagcacttggcagccacagggatcttctcaagaggaaaattggagggcatcacctccttgcttgaaactggccagtgcccattagactgaagaccacgcctctcagcattcgcctgactgtccagtaggccatcaacctcctctgttattggtttccccaacatcagtttatttcatcttcctaagccccagtggaatggatattctaagagttcctgcctcagtcaaagggcttgcttcagaatcaggagaacatgagttcaatgcccagaactatgttaatgacaaacaagcaaacagaccaacaaaatytgggggttggggggtggggggaggagaggcgggtgctgtgcacttgtaaccccagtgctgccaaaatagagcagtagggatctttggccaggtagcctaacctacttgttgagttctaggccactgagagattctgtgtttttttcttttnttctttttttctttttttccttttaaggtgaatagtgactgagaatagcatctgagtttgccctttggcttccatgaacatgtgcacccaagggcaccctcagctctaccacccctatatacaaacacacagagattgtatcttgaaggagatcgtctgtacagagtcttagcagaggaatctttaagggctgttttgtaagcatagttcaaagttcccgaattccagacactatgcttgggcaacagtcatttggaaaaacaagttcttggttgtctgtatccagcagttccactctggacatagacccaaaagaaatgaaaacagggccttgaagattttgttatgcttgcagtcatacaagcattatgtatgccaatctaggggcagaaaagagccacgtgtttatctatacatgaacaaataaaatgtggcatgcccattgagtgggatactattcagtcacaaaaagggaggaaattctgacacatgctctgtgtgtaaaagtccaggacattaagctaagtaggaaaggagcagacaccaaaatgacaaatgctatgtgattccagttatttctggcagtcaaatccttggatgtataaataaggatggtggttccaggtgttcttagcagcagctgccacctcctcttctttctcctcctcctcctcctcctcctcctcctcgttttctgcttcttttttcttttaacttatttatttattattgtatgtgtgtggcttcatgcatacacatgtgtcttggcacatgtaatagaagtgagaggacaactttcaagagttgcttctttccttctaccttgtagggattgaactcaggtcattagacttgactagcaagctcctctaccatctgagccatcttgctgaccctgcagccatcctaattggttggtgatgctgcatcattgtctcagattgtaattttctagtgattaatggcatcagatgactttttatgcgactgttggccattttatatctttggagagacagtaatttaatgtctttttcatttttaagagataaaacggagttgttttttttaagatttattttattgtgagtgttctggttgcatctgtgtctgtgtactacatgtgtgcctggtgcccatggaggccagaagagggtgatggatgccctggaactggggttactggtggttgtgagctaccttgtgggtgctggggactgatccacagtcctttggaagagagaagccagtaccttcactgccaaaccatctcttcttccacattactttgctccttcaaattgttgaggtcttggagtccccttatatatttaaactgcagtgctttatgaggcactgcccctcattttatagattgctgttttaaaatgttatgagtgtgtacagcatatgctggagcactgtggtacacgtgtagagggcagagaacaacttttgaaatgtctttctttccttccatcgtgtgagcagagctcaggttgggaaagcacctaccagtgatctcactgcctccctcccaccccccaccccccagccccaaactgcctttgtagtctgtcaaccattgtatcctatggccatagaaggtttttttggtttttgttttgttttgtttttttaagattgatgtaattctatttgtctgttttcacattcttccctgtgttttcttcggcatgtgtgtgagcgtagtatcacgtgactccattcatttttttttttttaaattttattttctttgcagtactgaggattgaacccagggcctgtgcaggctaggtgagtgctctaccaaccgtgccacagcatcagatccctattcactttttaaacttccttgccttgattgcccatatcttgctccctgctctatcacttccaggtaaagagcctgacttttctatggtgatgtgtggccattgagatagggtctcaccaagtcacccatgctggcctggaactcac

SEQ ID NO: 69 - DNA, 1082 bp, RAT
  modified_base (498)..(1082): N = A, C, G, T or U
cagtcttgataatcattcgattccagaatgggtggctgcattcctttcctaaaggcagcaaggacacggtgtcccagaatcatgccccttttgctgttgttgtctgccttcatttttttagtgtgcgtcctgggaggagccccaggacagaacccagaccgcagcaggatggacatggtatccatacacagcctctccgagctggagcgcctgaagctgcaagagactgcttaccacgaactcgtggccagacatttcctctctgaattcaagccggacagagttcaacccactgaccgtccaaacaccctgcagaagtggtttcagatgctgagaggccaagacagggctgcatcactcaagaccttcggtatccgcctggaagaagttctggtgaacgagcttacccgccgcaagcaacgggaactgacagacaacatgcaggttgaagatatcaacggttccactggacgtcgtcgtcggggaaacgtggtgcanaaaatggttggaccatgaagcgctttttcmgcaggnaggtaatgagscctactyctgccgtagagttcactacgccgtgggacgccgaggtgcaatttctgtggatagcgtagctgaactggagaatggagccctgctgctgcagatcctgcagctctcacagctttcattgcctattggccagcgacttctgggatccaaaaggaagatgagcctcaacccaattgctcaacaaatccctcacattgttgagacttgctgcaaattcattgagaaacatggcttaagctctgtggggattttcaccatcgagtactctctgcggagggtgattgagcttcgtgagttatttgacaaaggcatggatattgtcctggatgacagtgtgaatgtcaatgatgtggctgaactcctcaaggagtttwttcgtgagatgaaggactctctgctgcctgatgaactgtrcactgtccttcctcctgactgygactctgaaaccccangatcaggtttctgccctgcancttnngnncnccctgatgccaccntnctacaccgnnnccctnnnncnntgntgnnngnnn

SEQ ID NO: 70 - DNA, 1720 bp, RAT
  modified_base (1)..(1035): N = A, C, G, T or U
ngaggagggnnnnngngggnnggtggtnnnntntnnggnnnatgtgatggggttagnancatnnntgatarntgggrcatcctcttncaggtgcccccccatattcagaagcaggttgntaagcgtgtgtggaagtccagccctgaagccctggattttatcagacgccgcaatctgaggaagatccagagtgctcgcataaagatggaggaggacgctttactttctgatcctgtggacaactctgccgaagcccaggctgctatccttgctcagagccagccctttgatgaagaccccgaaggagccccagatgtgaactttggagatggcatgaactatgactttgaagatgactcagattttgaagacgaggaccatccggatcttgcagaggttccctatcttgatgtaattccaaacaacgaagataccgactcagatgctgatgaaatgccaggttcctactgaggagcccgctgtgcctgctggcactgcccgttsccrtgacaagattgaaggcgctrgtaacccccccaatgcagagcgcccattgccccgtgtgccccgggagaaggagggcaaatctggcactggcttctttccttagatgttttcccttctctaaggtgccggacaggggaaaagggtgggggtagacctggaatgtcacaggaaacagtagagtattgaagacaaaggactaaagataagatgataagatggagttccacctgatgctcgggtcaggatgggaattctaaatacactgacagccccattactggggatgcttggtatctttagctggtaaaagcagagatatttctgtgtcatgcccaggttccctggtgctaccttgccttctcttttacccctgatccaggctttctcccactacagcaaccccttcctttaattgatgttacatttgtggtaaacatactgtcccagcagaagnctcacaannctcgaggtgctttccctccccacaagggaattgcatgcttttcctgactttcctacccctcctcntaagagctcactggaaagaccctcaagaggcatgttagaatgttaggtcagcctactgacagctgacaaacaattaatactaaactatgtcatgccaactcatagccgtgtccccgcagcaactccaccgcctcagaaatttctcccctcaaattatttagaccaatggctcgtcaaacagccactccaaaaaaattctgtgcagttttgctcaggacatgccaacggggaaacctcaagtgtaagcctaactagcttttctggggtcaaaagttagaggaaaaaattagattttagtcctggacctgttttaaagacagctggtgttcacacctctgttgtcagcaaaacaactagttaggcacagacacatagttccaagtagttaaaatcacctgattacaaacactcttccttatcgtctctgtagatcctctatgcaggacggtacaaatttgcgggacttgctctggtaaaacacagatatgggttatgtctgacatccaaattataactgatatttatgggtaacaacagctaaggtccttagaatgaagaatgtattgtattgagggatagaaactgatcatacaatgggtaacaaccacagagctcgaagatttgtgattgttaaaacttctctggcatttcatcattaataaacatctgtgttgtgacagcagca

SEQ ID NO: 71 - DNA, 8253 bp, Artificial Sequence
  Description of Artificial Sequence  Synthetic Primer
cagccttgataatcatccgattccagaatgggtggctgcattccttttctgaaggcagcaagggcactgtgccccagaatcatgccccctttgctgttgttgtccgccttcatttttttagtgagtgtcttgggaggagccccaggacacaaccccgaccgcaggacgaagatggtatcgatacacagcctctctgagctggagcgtctgaagctgcaagagactgcttaccacgaactcgtggccagacatttcctctccgaattcaaacctgacagaggtaagctgtaccccggattgtggcatcctcgccttcgggagagtggaccctccgggcaggacggggcggattaggggtctggcagggtgggaggggctcacgcctgatctcggacttgatcagttctcctggcgtctggggaactgaagaagccggagttgaaggctgtttgttgaattgcggaagaggtaaaaattagttgagtttggtcgaaggctgggagttgagggggcagtaagacagacgggcagatgtcagggacagacaagtggacagagggccggtgaagagacagccgcagcaggtgcggtgcgagaatagggcagagcgagacggaagcgcagagggacactctggagagaactgggagagatagagacagcgcggtcgagagacagagagtagagggagatagcaagacagtgcgtgagagagagacgctcctagccgggcggtgaacgggagggcggtgcgggcgcgggggcggcggcaggaggtgcgcctctagcactgcccacctcagctggctgccggggagcgagcaggacgcggcgcaggacgaggttcgagtgcaccagggaatctcaggggtaagcgcgcggatttggcagcggagcccaggcggttagcgcggacccaggcgcgccccagaggtattggaaagtgctgggggtggcagcttcgctaggcgcgcaccgcgaacgcagcttgaattgagcgaagcaaagggcgaggagcccccgctggcagccactgatgccagattcggagtgcggggacagccaggtgtcgccgcttctcctaccgcagcgcagggagccgacggcaacctggtgcgagcgcgcccgcagtaacttggcgaccctcacccgcggtggcgctgcaggagagcccctccctcccctctcctgttgcgtgatctgggggcttcagagccccggaatttatgttttaagtgttgtattgagcgactacgggtgcgtagcaagatgcgcggtgcgcacgcggcgcattagattgtctggtttggttaaattcttggagtctgataagagtgagataggcttgtccttgttgttaggtggggaaactgaggctcatagaaagcaaattacttttcgcccgctgggcagagctcgcctctctgccgctccctccccctgctgcagatcagggcattgggatgttttagtcacttattaagtcctatttgtaagtgtatcttttcttttttctctttctgtagctctgcctattgaccgtccgaacaccttggataagtggtttctgattttgagaggacagcagaggggtgagggggctcttgtattttctttagaaaagaaatagaatgctaaccccctgtgtagtgtggccctggctattggagactgggtggcgtccactgtcccaggggacactctcctcagctcccaaaccccacagtgggtgatatcaggaaatggcgcagtagctgtaggtgcttggggcggtcctctcagaccttcctggtggactcaccccacacagatcaacccgaagaaatcgccctctttgtttcaaggtagtcacccccagggaaaatttaaggcagggacctcgagcctcgtcggggtggttgagtggagctgagtcctttcgccaaaccactgcggggtctttttggctagctccccagggaggggcgctgcggttctggagaggtggggttggctgcgggtggttgctgggaggagtgctacagcccctgcttcctcctgatttaagcgctgcgaggctttgacgtttctcattctttctctcgtggctccccagctgtatcacacaagacatttggcattagcctggaagaggtcctggtgaacgagtttacccgccgcaagcatcttgaactgacagccacgatgcaggttgaagaagccaccggtcaggctgcgggccgtcgtcggggaaacgtggtgcgaagggtgtttggccgcatccggcgctttttcagtcgcaggcggaatgagcccaccttgccccgggagttcactcgccgtgggcgtcgagtgagttaaaccctccaggtttcaggcaggggcctctcctgaggtatgcaggaatgtgaggccggaggatcaaggcctgtgccctcagaggcaggggggagctgaacacaatatgttgaagtggtgtccttggcttgccacaaagggaatgtatcttgtcctgaatgtcatacatacagtactcaggaaggtcctaattgactctctttatttcccattttaagataatctggatttctccacagggtgcagtgtctgtggatagtctggctgagctggaagacggagccctgctgctgcagaccctgcagctttcaaaaatttcctttccaattggccaacgacttctgggatccaaaaggaagatgagtctcaatccgattgcgaaacaaatcccccaggttgttgaggcttgctgccaattcattgaaaaacatggtaagggagctgcaaatgggtaaatcaaggaaggaagcaaagggaaaggagggatgttctggaaatggggggagaaaagaggccgaggatgaagggcttggataacattcccctgacacccagtggaggtcgcgatgcagtagggggtgaggggagaagagaaatgccatgggaaagggattcagagatgcttagcatcccctggtcttttctttcaggcttaagcgcagtggggatttttacccttgaatactccgtgcagcgagtgcgtcaggtaaattggctatgtttacatgtttgttcctccaataagaaagagcaggaggaggtggggtttccccatagacctgactacctggtggggggaggagcagggtcttctttgtagcccccaggcccaccatattgccagctacctccactgaatactggaacctgttgaaggaatgaccaagatggagctgtggtgggccaggcagacagtccctgtcttctccttttccttgagctccgtgaagaatttgatcaaggtctggatgtagtgctggatgacaatcagaatgtgcatgatgtggctgcactcctcaaggagtttttccgtgacatgaaggattctctgctgccagatgatctgtacatgtcattcctcctgacagcaagtgagtcttctgacctccctagtagggcaaggaaaggaaatattactggggtctagggggttctagggacagagctcaggctggaggactttgaagaactaaggcaaaatgaccataagaagattttaaaaacctctccagaatatatacaattattatttgtcaattaaaaataaaattttaataaaaaacttatttgggttactttctttgtatttctaaatgttccattataagtctgttgcttggaaatttctctagagcttcttgaagatgtgtgtgccctctcaggtcaaaaaaaaaaaaaaccacccaggttctgtagaggaggtctggtttttatgggggcacttcttgctttcagagttcctgagctcaaatgctttggaaatgagcaaaggcaagatgcctgggtactttctccataactgccagcaagtttcaagcagaatctcacctcccttggtatcagtctgcagccaagactatccccctgagacagcccctgttttccttcttctagctttaaagccccaggatcagctttctgccctgcagttgctggtctacctgatgccaccctgccacagtgataccctggagcgtctgctgaaggccctgcataaaatcactgagaactgcgaggactcaattggcattgatggacagttggtaaaaagatcttggaaagagtagtgaaaactgtagtaggggacatatgtgggagtatatcagcaagagggtggatggagaggagagagagaaacaagaataaaaagagacatattcatttgtacaactcccagtactacctcagcgtcaatcactttctgctttcctttgcctaggtcccaggcaaccgtatgacttccactaacttggccttggtgtttggatctgctctcctgaaaaaaggaaagtttggcaagagagagtccaggaaaacaaagctggggattgatcactatgttgcttctgtcaatgtggtccgtgccatgattgataactgggatgtcctcttccaggtaggtgcaagttttggatgcacttgttttacacatccatctcctgttcacaaggcaaggcacagttgtacctgcatctttgagggtagccaaaacaggcagccacaagatcgtccagtctagaaattcctcccacattagctggccctggtacctttagtcctaggtacctgaaagagatgagtataaatgaaaaggtgctgtgtaatgatggctaatgttgctaattctaccctcaggtgcctccccatattcagaggcaggttgctaagcgcgtgtggaagtccagcccggaagcacttgattttatcagacgcaggaacttgaggaagatccagtgagtgttttttgggtttgtttatgcttagcctgaagtacctcctcctatggggttttccaactccagactatctttatattaagtaacgcaagtacacaaactgactttttcaaaatccttcccattcaggagtgcacgcataaagatggaagaggatgcactactttctgatccagtggaaacctctgctgaagcccgggctgctgtccttgctcaaagcaagccttctgatgaaggtcagttccctgctggaggtcaggcccttgctgaaggtcagtcccttgttgaaggtcaggccctttttgaaggccaggtcgttgctgaagatgagccccttgaggaagatgagtcctctgaggaagatgaagagctagtatttgaatatcttaatcaaggagtagtctttggggaagctcaaggtctagaaatcccaaataactttgagattcagggcccacatcttgaaggaataccagagcttgatgaggaagatgatgatgatgataatgatactttaaattttgatgatcttcctccccttggagatattctagaaccagataatgaaattccagaactcattgagatcccagactttgaagaaattccatattttgatatggtcccagaccttgaaggagccccagatgtgcaagaaatcctgaaccctggagaaaactcaaaccatgacctagaagaaggagcagattttgaagaccaccaaaatcttaaccttgcagaagctccctatcttgatgtaatcccaaacaatgaaatcccagaccacgcagatgcttcagataatgatgaaatccaagattctgaagaagatcccagtcttgaagatgtcccagctgttgatgaagttccaaatgatgaagcctcaaatactgaagaaatcccagactatgaagccccagaggtaaatgggatttcagactctgaggaagaccttgattttgaagaagtctcagctctttatgtggttccaagccctgaagaagcccctggtggacatgaaatcccaaatcatgaagaatctccagaggttagtggactctcagaccctgaagaagatcccagtcttgaagaggtctcagattttgatggaatcccaaaatgatgaggaagcctcagacactgaagaaattccagaccatgaagaagctccagagaccaatggaattgcaggccctgaagaggtcccagctctccatgtagtcacaggccctgaaaatgtctctgattttgatgaaatgcaagaccatgaagaatcctcagatgtgagtggagtcccaaacaccgaagaagcctcagatattgaagaaattccgggacgtgaagaagcctctgatgttagtggaaaccaagactctgaagaaagccctagtcttgaagaagattcagttctctatgtggctccaaaacctaaggatgcccagatttacaacaaagttcaagattatcagaaagaccaagccagtgcatttgaagaagaagaagttaccactatggctccagaaactgatgatgtctccattctcaatacaattccagaccctccacaagacctggctgtcaatcttgaagaagtcatggatgtggaagaagtcccaatgcctaaagatttaaactctaaagaggttcagaatccaaaggcaattcagtttcataaagatgcagatgttgcattccctgtagataccactctccttaaaaacaagtctcagcctggccaagtcatggactacaagtcctcaccagaggtgttgaagggcaacacctgctttctgtcttccatgcagtgctgtaagctttcagctctagctttgttactcatttgcttggtaaaattagtcaggttacttgtagggtggtggtggttattaggaggtcgccctcttacctgtctccctggtggtattgtccattccctctgagcgcagggtatctgtcttactctttctctttcctaactcagctcttttcccagttcccacagtggtcctagattcagagatctgtgttctgctgtcaggctgtagtattcttatatgtcaaatggggataatcatccctgtcctaccttcttgcctcatactgtatttgaaactcaaaattaggtcttagtgccttgggtgctgcgcaaagtataaagtctagaaacataaggagttaacattgttactatttttcaggttcctctgaggagccagctgtgccttccggcactgcccgttcccatgacgatgaggaaggagcgggtaaccctcccattccggagcaagaccgcccattgctccgtgtgccccgggagaaggaggccaaaactggcgtcagctacttctttccttagatgtttttccttctataaggtgccagacaggggaaaagggtgggggtacatctgggatgtcacaggaaacattaaggagagagttgaaggtaaagatctgaaggtaagaaggagttccacctgatgctcgggtcaggatgagaattccaaacacactgccagccccttcactggggatgcttggtctcttctgctggtaaaagcagagatgtttctgtgtcatgcccaagctccccggtgctaccttgcctttctcttttacccctgatcttggctttctctctctctctgcagactttcctttaattgatgtgacatttgtggtaaacacctttcccagggaacctcacaaatcttgagatgctttcccttccccagatgggattgcatgatttccctgactttcctaccctcctccagagagctcagttggaaaggccctcaagaggcatgctagaacgttaggtcagcctactgacagctgacaaacaattaatgcgaaatcatgtcacaccaacccatagccgtgtccacgcagcaactccaccaccttaggatttccccctccaaattattcagaccaatggcttgccaaatggcctctcccaaaattctgtacagttttgctcaggtcacgccaacagggaaacctcaagtgtaggtctaattagtgtttctgggatccaaagttagaggaaaatttagattttattgcctggatctgctttaaagacaattggtgtttacaccctcttgtcagcaaaacagctagttaggtaaggacatatagttccaagtaggtaaagtcacttgattacaaatgttcttaactatcgtctctgtaattcctttatacaggacagtacaaaattgtgggacatgctctggtaacacacagatatgggttgcatatgatccagaattacagctgatattatggatgacaactgctaaggtccataaaatgaagactgtattgtattgagggatagaaattgatcatttaatgggtaacaactgctgagctcaaagatttgtgattgttaaaacttctctggcatttaatcattaataaacatctgtattgtgacagcagcatattca

SEQ ID NO: 72 - DNA, 2891 bp, Artificial Sequence
  Description of Artificial Sequence  Synthetic Primer
cagccttgataatcatccgattccagaatgggtggctgcattccttttctgaaggcagcaagggcactgtgccccagaatcatgccccctttgctgttgttgtccgccttcatttttttagtgagtgtcttgggaggagccccaggacacaaccccgaccgcaggacgaagatggtatcgatacacagcctctctgagctggagcgtctgaagctgcaagagactgcttaccacgaactcgtggccagacatttcctctccgaattcaaacctgacagaggtaagctgcctattgaccgtccgaacaccttggataagtggtttctgattttgagaggacagcagagggctgtatcacacaagacatttggcattagcctggaagaggtcctggtgaacgagtttacccgccgcaagcatcttgaactgacagccacgatgcaggttgaagaagccaccggtcaggctgcgggccgtcgtcggggaaacgtggtgcgaagggtgtttggccgcatccggcgctttttcagtcgcaggcggaatgagcccaccttgccccgggagttcactcgccgtgggggtgcagtgtctgtggatagtctggctgagctggaagacggagccctgctgctgcagaccctgcagctttcaaaaatttcctttccaattggccaacgacttctgggatccaaaaggaagatgagtctcaatccgattgcgaaacaaatcccccaggttgttgaggcttgctgccaattcattgaaaaacatgatggagctgtggtgggccaggcagacagtccctgtcttctccttttccttgagctccgtgaagaatttgatcaaggtctggatgtagtgctggatgacaatcagaatgtgcatgatgtggctgcactcctcaaggagtttttccgtgacatgaaggattctctgctgccagatgatctgtacatgtcattcctcctgacagcaagtttaaagccccaggatcagctttctgccctgcagttgctggtctacctgatgccaccctgccacagtgataccctggagcgtctgctgaaggccctgcataaaatcactgagaactgcgagggcaaccgtatgacttccactaacttggccttggtgtttggatctgctctcctgagagagtccaggaaaacaaagctggggattgatcactatgttgcttctgtcaatgtggtccgtgccatgattgataactgggatgtcctcttccagcaggtgcctccccatattcagaggcaggttgctaagcgcgtgtggaagtccagcccggaagcacttgattttatcagacgcaggaacttgaggaagatccagagtgcacgcataaagatggaagaggatgcactactttctgatccagtggaaacctctgctgaagcccgggctgctgtccttgctcaaagcaagccttctgatgaagaccttgaaggagccccagatgtgcaagaaatcctgaaccctggagaaaactcaaaccatgacctagaagaaggagcagattttgaagaccaccaaaatcttaaccttgcagaagctccctatcttgatgtaatcccaaacaatgaaatcccagaccacgcagatgcttcagataatgatgttcctctgaggagccagctgtgccttccggcactgcccgttcccatgacgatgaggaaggagcgggtaaccctcccattccggagcaagaccgcccattgctccgtgtgccccgggagaaggaggccaaaactggcgtcagctacttctttccttagatgtttttccttctataaggtgccagacaggggaaaagggtgggggtacatctgggatgtcacaggaaacattaaggagagagttgaaggtaaagatctgaaggtaagaaggagttccacctgatgctcgggtcaggatgagaattccaaacacactgccagccccttcactggggatgcttggtctcttctgctggtaaaagcagagatgtttctgtgtcatgcccaagctccccggtgctaccttgcctttctcttttacccctgatcttggctttctctctctctctgcagactttcctttaattgatgtgacatttgtggtaaacacctttcccagggaacctcacaaatcttgagatgctttcccttccccagatgggattgcatgatttccctgactttcctaccctcctccagagagctcagttggaaaggccctcaagaggcatgctagaacgttaggtcagcctactgacagctgacaaacaattaatgcgaaatcatgtcacaccaacccatagccgtgtccacgcagcaactccaccaccttaggatttccccctccaaattattcagaccaatggcttgccaaatggcctctcccaaaattctgtacagttttgctcaggtcacgccaacagggaaacctcaagtgtaggtctaattagtgtttctgggatccaaagttagaggaaaatttagattttattgcctggatctgctttaaagacaattggtgtttacaccctcttgtcagcaaaacagctagttaggtaaggacatatagttccaagtaggtaaagtcacttgattacaaatgttcttaactatcgtctctgtaattcctttatacaggacagtacaaaattgtgggacatgctctggtaacacacagatatgggttgcatatgatccagaattacagctgatattatggatgacaactgctaaggtccataaaatgaagactgtattgtattgagggatagaaattgatcatttaatgggtaacaactgctgagctcaaagatttgtgattgttaaaacttctctggcatttaatcattaataaacatctgtattgtgacagcagca

SEQ ID NO: 73 - protein, 629 aa, Artificial Sequence
  Description of Artificial Sequence  Synthetic Peptide
MGGCIPFLKAARALCPRIMPPLLLLSAFIFLVSVLGGAPGHNPDRRTKMVSIHSLSELERLKLQETAYHELVARHFLSEFKPDRGKLPIDRPNTLDKWFLILRGQQRAVSHKTFGISLEEVLVNEFTRRKHLELTATMQVEEATGQAAGRRRGNVVRRVFGRIRRFFSRRRNEPTLPREFTRRGGAVSVDSLAELEDGALLLQTLQLSKISFPIGQRLLGSKRKMSLNPIAKQIPQVVEACCQFIEKHDGAVVGQADSPCLLLFLELREEFDQGLDVVLDDNQNVHDVAALLKEFFRDMKDSLLPDDLYMSFLLTASLKPQDQLSALQLLVYLMPPCHSDTLERLLKALHKITENCEGNRMTSTNLALVFGSALLRESRKTKLGIDHYVASVNVVRAMIDNWDVLFQQVPPHIQRQVAKRVWKSSPEALDFIRRRNLRKIQSARIKMEEDALLSDPVETSAEARAAVLAQSKPSDEDLEGAPDVQEILNPGENSNHDLEEGADFEDHQNLNLAEAPYLDVIPNNEIPDHADASDNDVPLRSQLCLPALPVPMTMRKERVTLPFRSKTAHCSVCPGRRRPKLASATSFLRCFSFYKVPDRGKGWGYIWDVTGNIKERVEGKDLKVRRSST

SEQ ID NO: 74 - DNA, 2893 bp, Artificial Sequence
  modified_base (2134)..(2208): N = A, C, G, T or U
cagtcttgataatcattcgattccagaatgggtggctgcattcctttcctaaaggcagcaaggacacggtgtcccagaatcatgccccttttgctgttgttgtctgccttcatttttttagtgtgcgtcctgggaggagccccaggacagaacccagaccgcagcaggatggacatggtatccatacacagcctctccgagctggagcgcctgaagctgcaagagactgcttaccacgaactcgtggccagacatttcctctctgaattcaagccggacagagttcaacccactgaccgtccaaacaccctgcagaagtggtttcagatgctgagaggccaagacagggctgcatcactcaagaccttcggtatccgcctggaagaagttctggtgaacgagcttacccgccgcaagcaacgggaactgacagacaacatgcaggttgaagatatcaacggttccactggacgtcgtcgtcggggaaacgtggtgcagaaaatggttggacgcatgaagcgctttttcagcaggaaggtaatgagccctacttctgccgtagagttcactcgccgtgggacgcgaggtgcaatttctgtggatagcgtagctgaactggagaatggagccctgctgctgcagatcctgcagctctcacagctttcattgcctattggccagcgacttctgggatccaaaaggaagatgagcctcaacccaattgctcaacaaatccctcacattgttgagacttgctgcaaattcattgagaaacatggcttaagctctgtggggattttcaccatcgagtactctctgcggagggtgattgagcttcgtgagttatttgacaaaggcatggatattgtcctggatgacagtgtgaatgtcaatgatgtggctgaactcctcaaggagttttttcgtgagatgaaggactctctgctgcctgatgaactgtacatgtccttcctcctgactgcgactctgaaaccccaggatcaggtttctgccctgcagcttctggtctccctgatgccaccctgctacaccgataccctggagcgtctgctgaaggccctgcataaaatcactgagaactgcgagggcaaccgtatgacttccactaacttggccttggtgtttggatctgctctcctgagagagtccaggaaaacaaagctggggattgatcactatgttgcttctgtcaatgtggtccgtgccatgattgataactgggatgtcctcttccagcaggtgcccccccatattcagaagcaggttgctaagcgtgtgtggaagtccagccctgaagccctggattttatcagacgccgcaatctgaggaagatccagagtgctcgcataaagatggaggaggacgctttactttctgatcctgtggacaactctgccgaagcccaggctgctatccttgctcagagccagccctttgatgaagaccccgaaggagccccagatgtgaactttggagatggcatgaactatgactttgaagatgactcagattttgaagacgaggaccatccggatcttgcagaggttccctatcttgatgtaattccaaacaacgaagataccgactcagatgctgatggaaatgccaggttcctactgaggagcccgctgtgcctgctggcactgcccgttcccatgacaagattgaaggcgctggtaacccccccaatgcagagcgcccattgccccgtgtgccccgggagaaggagggcaaatctggcactggcttctttccttagatgttttcccttctctaaggtgccggacaggggaaaagggtgggggtacacctggaatgtcacaggaaacagtagagtatttgaagacaaaggacttaaagataagatgataagatggagttccacctgatgctcgggtcaggatgggaattctaaatacactgacagccccattactggggatgcttggtatctttagctggtaaaagcagagatatttctgtgtcatgcccaggttccctggtgctaccttgccttctcttttacccctgatccaggctttctcccactacagcaaccccttcctttaattgatgttacatttgtggtaaacatactgtcccagcagaagnctcacaannctcgaggtgctttccctccccacaagggaattgcatgcttttcctgactttcctacccctcctcntaagagctcactggaaagaccctcaagaggcatgttagaatgttaggtcagcctactgacagctgacaaacaattaatactaaactatgtcatgccaactcatagccgtgtccccgcagcaactccaccgcctcagaaatttctcccctcaaattatttagaccaatggctcgtcaaacagccactccaaaaaaattctgtgcagttttgctcaggacatgccaacggggaaacctcaagtgtaagcctaactagcttttctggggtcaaaagttagaggaaaaaattagattttagtcctggacctgttttaaagacagctggtgttcacacctctgttgtcagcaaaacaactagttaggcacagacacatagttccaagtagttaaaatcacctgattacaaacactcttccttatcgtctctgtagatcctctatgcaggacggtacaaatttgcgggacttgctctggtaaaacacagatatgggttatgtctgacatccaaattataactgatatttatgggtaacaacagctaaggtccttagaatgaagaatgtattgtattgagggatagaaactgatcatacaatgggtaacaaccacagagctcgaagatttgtgattgttaaaacttctctggcatttcatcattaataaacatctgtgttgtgacagcagca

SEQ ID NO: 75 - protein, 627 aa, Artificial Sequence
  Description of Artificial Sequence  Synthetic Peptide
MGGCIPFLKAARTRCPRIMPLLLLLSAFIFLVCVLGGAPGQNPDRSRMDMVSIHSLSELERLKLQETAYHELVARHFLSEFKPDRVQPTDRPNTLQKWFQMLRGQDRAASLKTFGIRLEEVLVNELTRRKQRELTDNMQVEDINGSTGRRRRGNVVQKMVGRMKRFFSRKVMSPTSAVEFTRRGTRGAISVDSVAELENGALLLQILQLSQLSLPIGQRLLGSKRKMSLNPIAQQIPHIVETCCKFIEKHGLSSVGIFTIEYSLRRVIELRELFDKGMDIVLDDSVNVNDVAELLKEFFREMKDSLLPDELYMSFLLTATLKPQDQVSALQLLVSLMPPCYTDTLERLLKALHKITENCEGNRMTSTNLALVFGSALLRESRKTKLGIDHYVASVNVVRAMIDNWDVLFQQVPPHIQKQVAKRVWKSSPEALDFIRRRNLRKIQSARIKMEEDALLSDPVDNSAEAQAAILAQSQPFDEDPEGAPDVNFGDGMNYDFEDDSDFEDEDHPDLAEVPYLDVIPNNEDTDSDADGNARFLLRSPLCLLALPVPMTRLKALVTPPMQSAHCPVCPGRRRANLALASFLRCFPFSKVPDRGKGWGYTWNVTGNSRVFEDKGLKDKMIRWSST